(19) United States
(12) Patent Application Publication
(10) Pub. No.: US 2020/0339587 A1
(43) Pub. Date: Oct. 29, 2020

(54) CRYSTALLINE SUBSTITUTED CYCLOHEXYL PYRAZOLO[1,5-A]PYRIMIDINYL CARBOXAMIDE COMPOUND AND THERAPEUTIC USES THEREOF

(71) Applicant: Lysosomal Therapeutics Inc., Cambridge, MA (US)

(72) Inventors: Renato T. Skerlj, West Newton, MA (US); Karel Marie Joseph Brands, Las Vegas, NV (US)

(21) Appl. No.: 16/955,589

(22) PCT Filed: Dec. 21, 2018
(86) PCT No.: PCT/US2018/067330

§ 371 Date: Jun. 18, 2020

Related U.S. Application Data
(60) Provisional application No. 62/608,652, filed on Dec. 21, 2017.

(51) Int. Cl. C07D 487/04

(52) U.S. Cl. CPC C07D 487/04; C07B 2200/13

(57) ABSTRACT

The invention provides crystalline 5,7-dimethyl-N-((1S*,4S)-4-(pentyloxy)cyclohexyl) pyrazolo[1,5-a]pyrimidine-3-carboxamide, compositions containing the crystalline compound, methods for making the crystalline compound, medical kits, and methods for using the crystalline compound and compositions to treat a medical disorder, e.g., Gaucher disease, Parkinson's disease, Lewy body disease, dementia, or multiple system atrophy, in a patient.

### CROSS-REFERENCE TO RELATED APPLICATION

This application claims the benefit of, and priority to, U.S. Provisional Application No. 62/608,652, filed Dec. 21, 2017, the disclosure of which is incorporated herein by reference in its entirety.

### FIELD OF THE INVENTION

The invention provides crystalline 5,7-dimethyl-N-((1S*, 4S)-4-(pentyloxy)cyclohexyl)pyrazolo[1,5-a]pyrimidine-3-carboxamide, compositions containing the crystalline compound, methods for making the crystalline compound, medical kits, and methods of using the crystalline compound and compositions to treat medical disorders in a patient.

### BACKGROUND

Gaucher disease is a genetic disorder associated with a deficiency of the lysosomal enzyme, glucocerebrosidase. Gaucher disease has been reported to have an incidence of approximately 1 in 20,000 live births in the general population, and it is a common lysosomal storage disorder. Current treatments for patients suffering from this disease include enzyme replacement therapy, which tends to be expensive, analgesics for bone pain relief, and medical procedures such as blood and platelet transfusions, splenectomy, and joint replacement for patients who experience bone erosion. However, new treatment options are needed with improved efficacy across a broader range of patients and/or reduced adverse side effects.

Mutations in the gene encoding glucocerebrosidase are also a risk factor for Parkinson's disease and diffuse Lewy Body Disease. Parkinson's disease is a degenerative disorder of the central nervous system associated with death of dopamine-containing cells in a region of the midbrain. Parkinson's disease afflicts millions of people, and the incidence of the disease increases with age. Treatment of Parkinson's disease frequently involves use of levodopa and dopamine agonists. However, these drugs can produce significant side effects such as hallucinations, insomnia, nausea, and constipation. In addition, patients often develop tolerance to these drugs such that the drugs become ineffective at treating the symptoms of the disease, while sometimes also producing a movement disorder side effect called dyskinesia. Diffuse Lewy Body disease is a dementia that is sometimes confused with Alzheimer's disease.

Despite the advances made to date, there still remains a need for new therapeutic agents for treating Gaucher disease, Parkinson's disease, and related medical disorders. The present invention addresses these needs and provides other related advantages.

### SUMMARY

The invention provides crystalline 5,7-dimethyl-N-((1S*, 4S)-4-(pentyloxy)cyclohexyl)pyrazolo[1,5-a]pyrimidine-3-carboxamide, compositions containing the crystalline compound, methods for making the crystalline compound, medical kits, and methods of using the crystalline compound and compositions to treat medical disorders, e.g., Gaucher disease, Parkinson's disease, Lewy body disease, dementia, multiple system atrophy, epilepsy, bipolar disorder, schizophrenia, an anxiety disorder, major depression, polycystic kidney disease, type 2 diabetes, open angle glaucoma, multiple sclerosis, endometriosis, and multiple myeloma, in a patient. Various aspects and embodiments of the invention are described in further detail below.

Accordingly, one aspect of the invention provides a compound in crystalline form having the following formula:

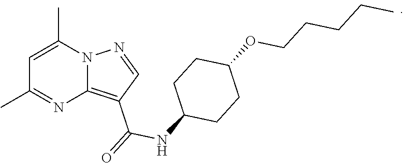 [SMILES: CCCCCO[C@H]1CC[C@H](NC(=O)c2cnn3c(C)cc(C)nc23)CC1]

Furthermore, additional compounds, including a crystalline polymorphic Form A, a crystalline polymorphic Form B, a crystalline polymorphic Form C and a crystalline hydrate Form D of 5,7-dimethyl-N-((1S*,4S)-4-(pentyloxy)cyclohexyl)pyrazolo[1,5-a]pyrimidine-3-carboxamide are described below.

Another aspect of the invention provides a pharmaceutical composition, comprising a pharmaceutically acceptable carrier and a compound described herein, such as a crystalline 5,7-dimethyl-N-((1S*,4S)-4-(pentyloxy)cyclohexyl)pyrazolo[1,5-a]pyrimidine-3-carboxamide. In certain embodiments, the crystalline compound is polymorphic Form A. In certain other embodiments, the crystalline compound is polymorphic Form B. In certain other embodiments, the crystalline compound is polymorphic Form C. In certain other embodiments, the crystalline compound is hydrate Form D.

Another aspect of the invention provides a method of treating a disorder, e.g., Gaucher disease, Parkinson's disease, Lewy body disease, dementia, multiple system atrophy, epilepsy, bipolar disorder, schizophrenia, an anxiety disorder, major depression, polycystic kidney disease, type 2 diabetes, open angle glaucoma, multiple sclerosis, endometriosis, and multiple myeloma, in a patient. The method comprises administering to a patient in need thereof a therapeutically effective amount of a compound described herein, such as a crystalline 5,7-dimethyl-N-((1S*,4S)-4-(pentyloxy)cyclohexyl)pyrazolo[1,5-a]pyrimidine-3-carboxamide, to treat the disorder, e.g., Gaucher disease, Parkinson's disease, Lewy body disease, dementia, multiple system atrophy, epilepsy, bipolar disorder, schizophrenia, an anxiety disorder, major depression, polycystic kidney disease, type 2 diabetes, open angle glaucoma, multiple sclerosis, or multiple myeloma. In certain embodiments, the crystalline compound is polymorphic Form A. In certain other embodiments, the crystalline compound is polymorphic Form B. In certain other embodiments, the crystalline compound is polymorphic Form C. In certain other embodiments, the crystalline compound is hydrate Form D. In certain embodiments, the disorder is Parkinson's disease.

Another aspect of the invention provides methods for making intermediate compounds used in the synthesis of 5,7-dimethyl-N(1S*,4S)-4-(pentyloxy)cyclohexyl)pyrazolo[1,5-a]pyrimidine-3-carboxamide. One intermediate is produced by a method comprising:
(a) admixing a compound of Formula (I), a base, and a solvent to produce a reaction mixture; wherein Formula (I) is represented by:

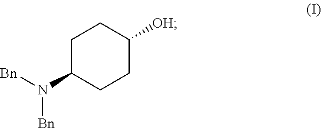 [SMILES: O[C@H]1CC[C@H](N(Cc2ccccc2)Cc2ccccc2)CC1]

(b) adding a n-pentyl alkylating agent to the reaction mixture to produce a compound of Formula (II):

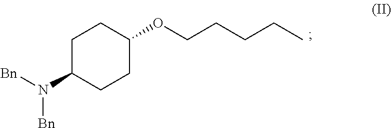 [SMILES: CCCCCO[C@H]1CC[C@H](N(Cc2ccccc2)Cc2ccccc2)CC1]

(c) exposing the compound of Formula (II) to acid HX to provide a compound of Formula (III):

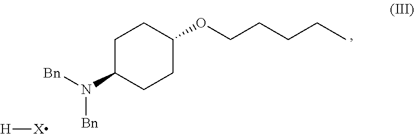

where X is anion; and
(d) exposing the compound of Formula (III) to hydrogenation conditions, to provide a compound of Formula (IV):

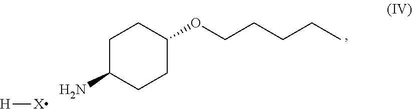

wherein X is an anion. Additional embodiments of the foregoing method are described in the detailed description.

An alternative method for producing the intermediate comprises:
(a) admixing a compound of Formula (I), a base, and a solvent to produce a reaction mixture; wherein Formula (I) is represented by:

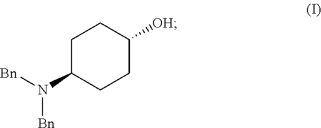 [SMILES: O[C@H]1CC[C@H](N(Cc2ccccc2)Cc2ccccc2)CC1]

(b) adding a n-pentyl alkylating agent to the reaction mixture to produce a compound of Formula (II):

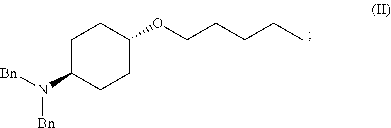 [SMILES: CCCCCO[C@H]1CC[C@H](N(Cc2ccccc2)Cc2ccccc2)CC1]

(c) exposing the compound of Formula (II) to hydrogenation conditions to provide a compound of Formula (II-a):

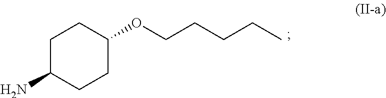

and
(d) exposing the compound of Formula (I-a) to an acid HX to provide a compound of Formula (IV):

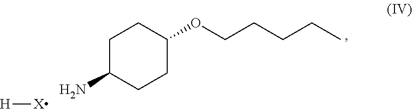

wherein X is an anion. Additional embodiments of the foregoing method are described in the detailed description.

In a procedure for producing a compound of Formula (VIII)

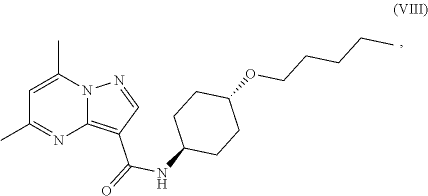 [SMILES: CCCCCO[C@H]1CC[C@H](NC(=O)c2cnn3c(C)cc(C)nc23)CC1]

namely 5,7-dimethyl-N-((1S*,4S)-4-(pentyloxy)cyclohexyl)pyrazolo[1,5-a]pyrimidine-3-carboxamide, the method comprises admixing a compound of Formula (VII) with an amide coupling reagent in the presence of a solvent (S1) to form an amide-coupling reaction mixture, and thereafter adding a compound of Formula (IV) to the amide-coupling reaction mixture, to provide the compound of Formula (VIII), wherein the compound of Formula (IV) is represented by

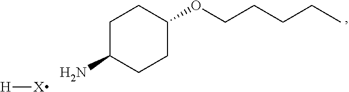

and the compound of Formula (VII) is represented by

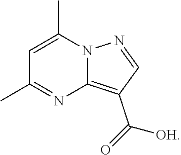

Additional embodiments of the foregoing method are described in the detailed description.

### BRIEF DESCRIPTION OF THE DRAWINGS

FIG. 1 is an X-ray powder diffractogram of crystalline polymorphic Form B of 5,7-dimethyl-N-((1S*, 4S)-4-(pentyloxy)cyclohexyl)pyrazolo[1,5-a]pyrimidine-3-carboxamide, as further described in Example 6.

FIG. 2 is a differential scanning calorimetry (DSC) curve of crystalline polymorphic Form B of 5,7-dimethyl-N-((1S*,4S)-4-(pentyloxy)cyclohexyl)pyrazolo[1,5-a]pyrimidine-3-carboxamide, as further described in Example 6.

FIG. 3 is an X-ray powder diffractogram of crystalline polymorphic Form B of 5,7-dimethyl-N-((1S*, 4S)-4-(pentyloxy)cyclohexyl)pyrazolo[1,5-a]pyrimidine-3-carboxamide, as further described in Example 7.

FIG. 4 is a differential scanning calorimetry (DSC) curve of crystalline polymorphic Form B of 5,7-dimethyl-N-((1S*,4S)-4-(pentyloxy)cyclohexyl)pyrazolo[1,5-a]pyrimidine-3-carboxamide, as further described in Example 7.

FIG. 5 is an X-ray powder diffractogram of crystalline polymorphic Form A of 5,7-dimethyl-N-((1S*,4S)-4-(pentyloxy)cyclohexyl)pyrazolo[1,5-a]pyrimidine-3-carboxamide, as further described in Example 9.

FIG. 6 is a differential scanning calorimetry curve (DSC) of crystalline polymorphic Form A of 5,7-dimethyl-N-((1S*,4S)-4-(pentyloxy)cyclohexyl)pyrazolo[1,5-a]pyrimidine-3-carboxamide, as further described in Example 9.

FIG. 7 is an X-ray powder diffractogram of crystalline polymorphic Form A of 5,7-dimethyl-N-((1S*,4S)-4-(pentyloxy)cyclohexyl)pyrazolo[1,5-a]pyrimidine-3-carboxamide, as further described in Example 10.

FIG. 8 is a differential scanning calorimetry curve (DSC) of crystalline polymorphic Form A of 5,7-dimethyl-N-((1S*,4S)-4-(pentyloxy)cyclohexyl)pyrazolo[1,5-a]pyrimidine-3-carboxamide, as further described in Example 10.

FIG. 9 is a thermal gravimetric analysis (TGA) curve of crystalline polymorphic Form A of 5,7-dimethyl-N-((1S*,4S)-4-(pentyloxy)cyclohexyl)pyrazolo[1,5-a]pyrimidine-3-carboxamide, as further described in Example 9.

FIG. 10 is an X-ray powder diffractogram of crystalline polymorphic Form C of 5,7-dimethyl-N-((1S*,4S)-4-(pentyloxy)cyclohexyl)pyrazolo[1,5-a]pyrimidine-3-carboxamide, as further described in Example 11.

FIG. 11 is a differential scanning calorimetry curve (DSC) of crystalline polymorphic Form C of 5,7-dimethyl-N-((1S*,4S)-4-(pentyloxy)cyclohexyl)pyrazolo[1,5-a]pyrimidine-3-carboxamide, as further described in Example 11.

FIG. 12 is an X-ray powder diffractogram of crystalline hydrate Form D of 5,7-dimethyl-N-((1S*,4S)-4-(pentyloxy)cyclohexyl)pyrazolo[1,5-a]pyrimidine-3-carboxamide, as further described in Example 12.

FIG. 13 is a differential scanning calorimetry curve (DSC) of crystalline hydrate Form D of 5,7-dimethyl-N-((1S*,4S)-4-(pentyloxy)cyclohexyl)pyrazolo[1,5-a]pyrimidine-3-carboxamide, as further described in Example 12.

### DETAILED DESCRIPTION

The invention provides crystalline 5,7-dimethyl-N-((1S*,4S)-4-(pentyloxy)cyclohexyl)pyrazolo[1,5-a]pyrimidine-3-carboxamide, compositions containing the crystalline compound, methods for making the crystalline compound, medical kits, and methods of using the crystalline compound and compositions to treat medical disorders, e.g., Gaucher disease, Parkinson's disease, Lewy body disease, dementia, multiple system atrophy, epilepsy, bipolar disorder, schizophrenia, an anxiety disorder, major depression, polycystic kidney disease, type 2 diabetes, open angle glaucoma, multiple sclerosis, endometriosis, and multiple myeloma, in a patient. The practice of the present invention employs, unless otherwise indicated, conventional techniques of organic chemistry, pharmacology, cell biology, and biochemistry. Such techniques are explained in the literature, such as in “Comprehensive Organic Synthesis” (B. M. Trost & I. Fleming, eds., 1991-1992); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety. Various aspects of the invention are set forth below in sections; however, aspects of the invention described in one particular section are not to be limited to any particular section.

### I. Definitions

To facilitate an understanding of the present invention, a number of terms and phrases are defined below.

The terms “a” and “an” as used herein mean “one or more” and include the plural unless the context is inappropriate.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The abbreviation “Bn” as used herein refers to benzyl, which has the formula:

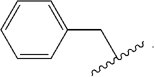 [SMILES: *Cc1ccccc1]

The symbol “ [symbol] ” indicates a point of attachment.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Further, enantiomers can be separated using supercritical fluid chromatographic (SFC) techniques described in the literature. Still further, stereoisomers can be obtained from stereomerically-pure intermediates, reagents, and catalysts by well-known asymmetric synthetic methods.

Geometric isomers can also exist in the compounds of the present invention. The symbol [symbol] denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ⁴C, ¹⁵N, ¹⁸O, ¹⁷O, ³¹P, ³²P, ³⁵S, ¹⁸F, and ³⁶Cl, respectively.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in, e.g., the Examples herein by substituting an isotopically labeled reagent for a non-isotopically labeled reagent.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

Abbreviations as used herein include O-(7-azabenzotriazol-1-yl)-N,N,N′,N′-tetramethyluronium hexafluorophosphate (HATU); 1-hydroxybenzotriazole (HOBt); 1-hydroxy-7-azabenzotriazole (HOAt); 2-hydroxypyridine-N-oxide (HOPO); 1-ethyl-3-(3-dimethylaminopropyl)carbodiimide hydrochloride (EDC); diisopropylethylamine (DIPEA); dimethylformamide (DMF); dimethylacetamide (DMA); methylene chloride (DCM); tert-butoxycarbonyl (Boc); tetrahydrofuran (THF); trifluoroacetic acid (TFA); N-methylmorpholine (NMM); triethylamine (TEA); Boc anhydride ((Boc)₂O); dimethylsulfoxide (DMSO); methyl ethyl ketone (MEK); methyl isobutyl ketone (MIBK); ethyl acetate (EtOAc); methyl tert-butyl ether (MTBE); flash column chromatography (FCC); and supercritical fluid chromatography (SFC); X-ray powder diffractogram (XRPD); differential scanning calorimetry (DSC).

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. Crystalline Substituted Cyclohexyl Pyrazolo[1,5-A]Pyrimidinyl Carboxamide Compounds

One aspect of the invention provides crystalline substituted cyclohexyl pyrazolo[1,5-a]pyrimidinyl carboxamide compounds. The crystalline substituted cyclohexyl pyrazolo[1,5-a]pyrimidinyl carboxamide compounds are contemplated to be useful in the methods, compositions, and kits described herein. In certain embodiments, the crystalline substituted cyclohexyl pyrazolo[1,5-a]pyrimidinyl carboxamide compound is a compound in crystalline form having the following formula:

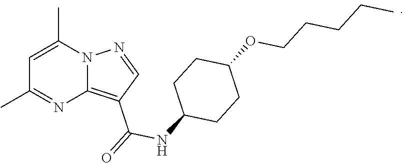 [SMILES: CCCCCO[C@H]1CC[C@H](NC(=O)c2cnn3c(C)cc(C)nc23)CC1]

The foregoing compound in crystalline form may be further characterized according to a particular crystalline form. In certain embodiments, the compound is crystalline polymorphic Form A. In certain other embodiments, the compound is crystalline polymorphic Form B. In certain other embodiments, the compound is crystalline polymorphic Form C. In certain other embodiments, the compound is crystalline hydrate Form D. Each are described in more detailed below.

### A. Crystalline Polymorphic Form A

In certain embodiments, the invention provides a compound in crystalline polymorphic Form A having the following formula:

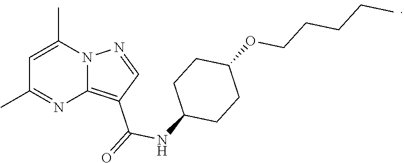 [SMILES: CCCCCO[C@H]1CC[C@H](NC(=O)c2cnn3c(C)cc(C)nc23)CC1]

In certain embodiments, such a compound in crystalline form may be characterized by an X-ray powder diffraction pattern comprising peaks at the following diffraction angles (2θ): 5.7±0.2, 11.5±0.2, 11.8±0.2, and 12.8±0.2. In certain embodiments, such a compound in crystalline form may be characterized by an X-ray powder diffraction pattern comprising peaks at the following diffraction angles (2θ): 5.7±0.2, 11.5±0.2, 11.8±0.2, 12.8±0.2, 17.2±0.2, 18.7±0.2, 19.6±0.2, 22.3±0.2, and 27.3±0.2.

In certain embodiments, the compound in crystalline polymorphic Form A is characterized by the X-ray powder diffraction pattern expressed in terms of diffraction angle 2θ, and optionally inter-planar distances d, and relative intensity (expressed as a percentage with respect to the most intense peak) as set forth in Table 1.

TABLE 1
X-RAY POWDER DIFFRACTOGRAM DATA OF CRYSTALLINE
POLYMORPHIC FORM A.

| Angle [2θ] | d-spacing [Å] | Relative Intensity [%] |
|---|---|---|
| 5.7 | 15.5 | 61.3 |
| 10.9 | 8.1 | 11.2 |
| 11.5 | 7.7 | 29.8 |
| 11.8 | 7.5 | 40.0 |
| 12.8 | 6.9 | 32.8 |
| 13.6 | 6.5 | 26.9 |
| 14.4 | 6.1 | 25.7 |
| 14.7 | 6.0 | 29.0 |
| 15.8 | 5.6 | 18.4 |
| 17.2 | 5.2 | 80.3 |
| 17.9 | 5.0 | 9.9 |
| 18.7 | 4.7 | 100.0 |
| 19.6 | 4.5 | 65.6 |
| 19.9 | 4.5 | 20.5 |
| 20.2 | 4.4 | 13.5 |
| 21.8 | 4.1 | 17.9 |
| 22.3 | 4.0 | 42.1 |
| 22.9 | 3.9 | 11.4 |
| 24.2 | 3.7 | 20.5 |
| 24.8 | 3.6 | 16.4 |
| 27.3 | 3.3 | 24.9. |

In certain embodiments, the relative intensity of the peak at said diffraction angles (2θ) is at least 20% with respect to the most intense peak in the X-ray powder diffraction pattern.

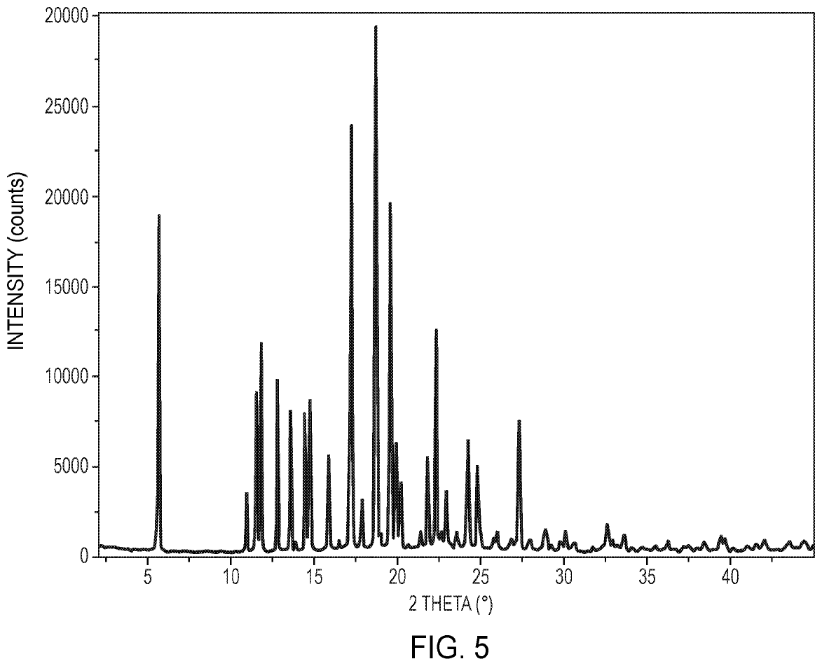

In yet other embodiments, the compound in crystalline polymorphic Form A is characterized by an X-ray powder diffraction pattern substantially the same as shown in FIG. 5.

In certain embodiments, such compound in crystalline form may be characterized by an X-ray powder diffraction pattern comprising peaks at the following diffraction angles (2θ): 5.7±0.2, 12.8±0.2, 14.4±0.2, and 17.1±0.2. In certain embodiments, such compound in crystalline form may be characterized by an X-ray powder diffraction pattern comprising peaks at the following diffraction angles (2θ): 5.7±0.2, 12.8±0.2, 14.4±0.2, 17.1±0.2, 22.3±0.2, 23.0±0.2 and 27.2±0.2.

In certain embodiments, the compound in crystalline polymorphic Form A is characterized by the X-ray powder diffraction pattern expressed in terms of diffraction angle 20 and optionally relative intensity (expressed as a percentage with respect to the most intense peak) as set forth in Table 2.

TABLE 2
X-RAY POWDER DIFFRACTOGRAM DATA OF CRYSTALLINE
POLYMORPHIC FORM A.

| Angle (2θ°) | Intensity % |
|---|---|
| 5.7 | 100 |
| 11.7 | 3.5 |
| 12.8 | 19.5 |
| 13.5 | 5.9 |
| 14.4 | 27.8 |
| 14.7 | 4.3 |
| 15.8 | 1.8 |
| 17.1 | 67.4 |
| 17.8 | 3.7 |
| 18.6 | 6.7 |
| 19.5 | 8.7 |
| 20.1 | 2.9 |
| 21.7 | 4.1 |
| 22.3 | 49.9 |
| 23.0 | 65.7 |
| 23.5 | 3.6 |
| 24.2 | 15.7 |
| 24.7 | 4.3 |
| 25.6 | 3.4 |
| 26.8 | 3.1 |
| 27.2 | 32.8 |
| 28.8 | 6.3 |
| 30.5 | 3.6 |
| 32.2 | 13.4 |
| 32.6 | 6.5 |

In certain embodiments, the pharmaceutical composition is further characterized by the feature that the relative intensity of the peak at said diffraction angles (2θ) is at least 20% with respect to the most intense peak in the X-ray powder diffraction pattern.

In certain embodiments, the compound in crystalline polymorphic Form A exists in a monoclinic crystal system and has a P2₁/c space group. In certain embodiments, the compound in crystalline polymorphic Form A is characterized by the crystallographic unit cell parameters as set forth in Table 3.

TABLE 3
UNIT CELL PARAMETERS OF CRYSTALLINE POLYMORPHIC
FORM A.

| | | |
|---|---|---|
| Unit cell | a = 15.8710(5) Å | α = 90° |
| dimensions | b = 9.4329(2) Å | β = 108.628(3)° |
| | c = 13.8255(4) | γ = 90° |

| | |
|---|---|
| Volume | 1961.38(10) Å³ |
| Z | 4 |
| Density | 1.214 Mg/m³. |
| (calculated) | |

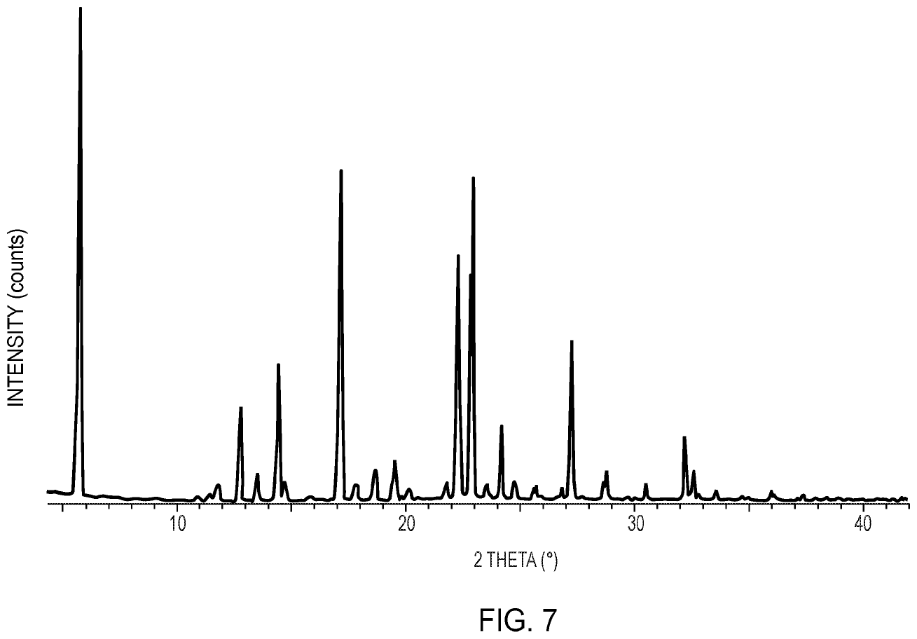

In other embodiments, the compound in crystalline polymorphic Form A is characterized by an X-ray powder diffraction pattern substantially the same as shown in FIG. 7.

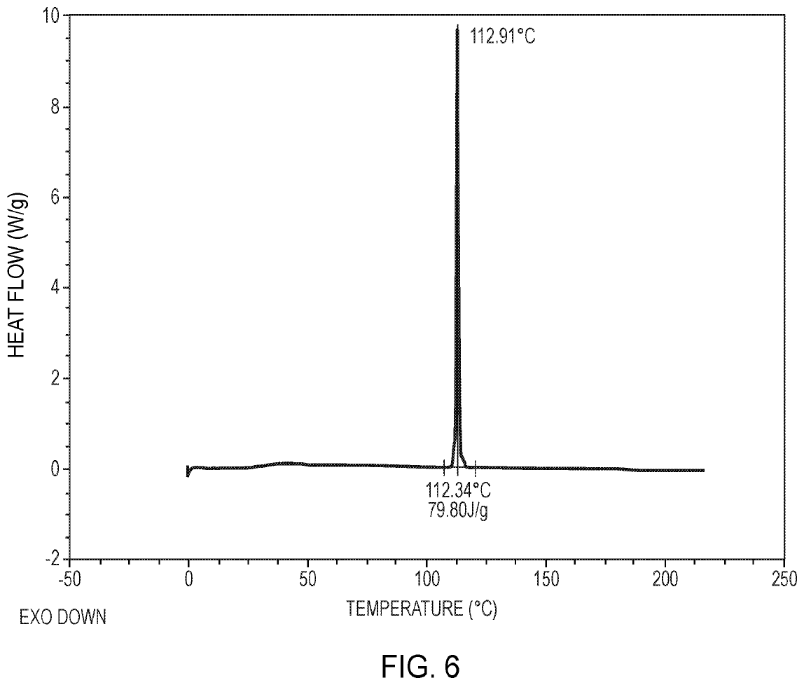

The compound in crystalline polymorphic Form A may also be characterized according to the temperature of melting point onset. Accordingly, in certain embodiments, the compound has a melting point onset as determined by differential scanning calorimetry in the range of from about 110 degrees Celsius to about 114 degrees Celsius, for example, at about 112 degrees Celsius. In yet other embodiments, the compound has a differential scanning calorimetry curve substantially the same as shown in FIG. 6.

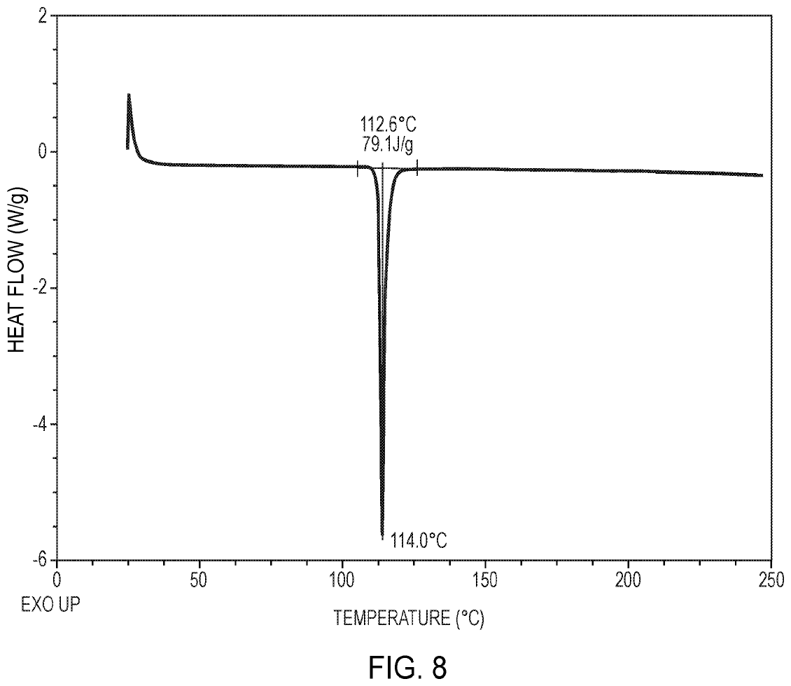

In certain embodiments, the compound has a melting point onset as determined by differential scanning calorimetry in the range of from about 112 degrees Celsius to about 116 degrees Celsius, for example, at about 114 degrees Celsius. In yet other embodiments, the compound has a differential scanning calorimetry curve substantially the same as shown in FIG. 8.

### B. Crystalline Polymorphic Form B

In certain embodiments, the invention provides a compound in crystalline polymorphic Form B having the following formula:

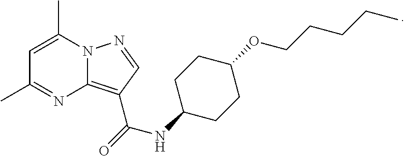 [SMILES: CCCCCO[C@H]1CC[C@H](NC(=O)c2cnn3c(C)cc(C)nc23)CC1]

In certain embodiments, the compound in crystalline form may be characterized by an X-ray powder diffraction pattern comprising peaks at the following diffraction angles (2θ): 4.0±0.2, 10.9±0.2, 12.3±0.2, and 16.2±0.2. In certain embodiments, the compound in crystalline form may be characterized by an X-ray powder diffraction pattern comprising peaks at the following diffraction angles (2θ): 4.0±0.2, 10.9±0.2, 12.3±0.2, 16.2±0.2, 20.2±0.2, 21.1±0.2, 21.5±0.2, 24.7±0.2, 27.6±0.2.

In certain embodiments, the compound in crystalline polymorphic Form B is characterized by the X-ray powder diffraction pattern expressed in terms of diffraction angle 2θ, and optionally inter-planar distances d, and relative intensity (expressed as a percentage with respect to the most intense peak) as set forth in Table 4.

TABLE 4
X-RAY POWDER DIFFRACTOGRAM DATA OF CRYSTALLINE
POLYMORPHIC FORM B.

| Angle [2θ] | d-spacing [Å] | Relative Intensity [%] |
|---|---|---|
| 4.0 | 21.9 | 100.0 |
| 10.9 | 8.2 | 89.0 |
| 11.4 | 7.8 | 22.4 |
| 12.1 | 7.3 | 18.4 |
| 12.3 | 7.2 | 73.2 |
| 13.4 | 6.6 | 17.7 |
| 16.0 | 5.5 | 13.9 |
| 16.2 | 5.5 | 32.5 |
| 16.5 | 5.4 | 11.6 |
| 17.8 | 5.0 | 11.7 |
| 18.6 | 4.8 | 19.7 |
| 19.5 | 4.6 | 13.2 |
| 20.2 | 4.4 | 88.0 |
| 20.4 | 4.3 | 11.1 |
| 21.1 | 4.2 | 48.3 |
| 21.5 | 4.1 | 58.8 |
| 21.6 | 4.1 | 27.1 |
| 22.3 | 4.0 | 9.8 |
| 22.5 | 4.0 | 21.2 |
| 22.9 | 3.9 | 21.2 |
| 23.4 | 3.8 | 36.5 |
| 23.5 | 3.8 | 33.3 |
| 24.7 | 3.6 | 35.1 |
| 25.7 | 3.5 | 11.6 |
| 25.8 | 3.5 | 10.1 |
| 27.6 | 3.2 | 27.9 |
| 30.8 | 2.9 | 8.5 |
| 30.9 | 2.9 | 12.4. |

In certain embodiments, the relative intensity of the peak at said diffraction angles (2θ) is at least 20% with respect to the most intense peak in the X-ray powder diffraction pattern.

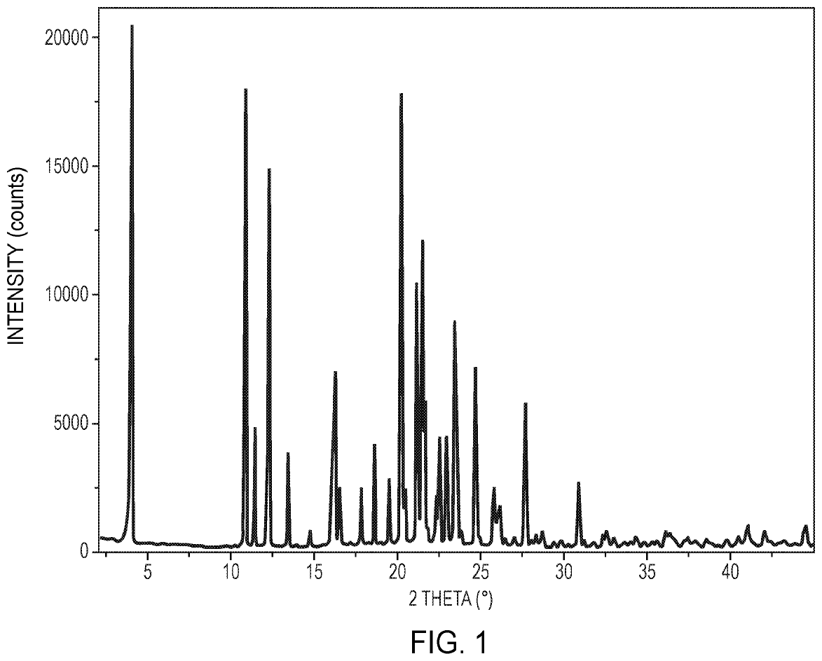

In yet other embodiments, the compound in crystalline polymorphic Form B is characterized by an X-ray powder diffraction pattern substantially the same as shown in FIG. 1.

In certain embodiments, the compound in crystalline form may be characterized by an X-ray powder diffraction pattern comprising peaks at the following diffraction angles (2θ): 4.2±0.2, 10.9±0.2, 11.5±0.2, and 12.4±0.2. In certain embodiments, the compound in crystalline form may be characterized by an X-ray powder diffraction pattern comprising peaks at the following diffraction angles (2θ): 4.2±0.2, 10.9±0.2, 11.5±0.2, 12.4±0.2, 16.3±0.2, 21.5±0.2, 22.3±0.2, 22.4±0.2, 22.9±0.2 and 23.0±0.2.

In certain embodiments, the compound in crystalline polymorphic Form B is characterized by the X-ray powder diffraction pattern expressed in terms of diffraction angle 2θ and optionally relative intensity (expressed as a percentage with respect to the most intense peak) as set forth in Table 5.

TABLE 5
X-RAY POWDER DIFFRACTOGRAM DATA OF CRYSTALLINE
POLYMORPHIC FORM B.

| Angle (2θ°) | Intensity % |
|---|---|
| 4.2 | 51.0 |
| 10.9 | 100 |
| 11.5 | 31.2 |
| 12.4 | 96.2 |
| 13.5 | 6.5 |
| 14.8 | 5.5 |
| 16.3 | 51.0 |
| 17.7 | 4.1 |
| 18.6 | 2.1 |
| 19.5 | 2.2 |
| 20.2 | 9.9 |
| 20.4 | 7.9 |
| 21.1 | 10.5 |
| 21.5 | 23.7 |
| 21.8 | 5.5 |
| 22.3 | 27.1 |
| 22.4 | 40.4 |
| 22.9 | 30.2 |
| 23.0 | 28.6 |
| 23.5 | 7.8 |
| 23.8 | 4.4 |
| 24.7 | 4.9 |
| 25.8 | 19.1 |
| 27.6 | 6.6 |
| 28.3 | 2.7 |
| 29.8 | 2.4 |

In certain embodiments, the pharmaceutical composition is further characterized by the feature that the relative intensity of the peak at said diffraction angles (2θ) is at least 20% with respect to the most intense peak in the X-ray powder diffraction pattern.

In certain embodiments, the compound in crystalline polymorphic Form B exists in a monoclinic crystal system and has a P2₁/c space group. In certain embodiments, the compound in crystalline polymorphic Form B is characterized by the crystallographic unit cell parameters as set forth in Table 6.

TABLE 6
UNIT CELL PARAMETERS OF CRYSTALLINE POLYMORPHIC
FORM B.

| | | |
|---|---|---|
| Unit cell | a = 5.49080(10) Å | α = 90° |
| dimensions | b = 43.1070(8) Å | β = 94.827(2)° |
| | c = 8.2570(2) | γ = 90° |

| | |
|---|---|
| Volume | 1947.43(7) Å³ |
| Z | 4 |
| Density | 1.223 Mg/m³. |
| (calculated) | |

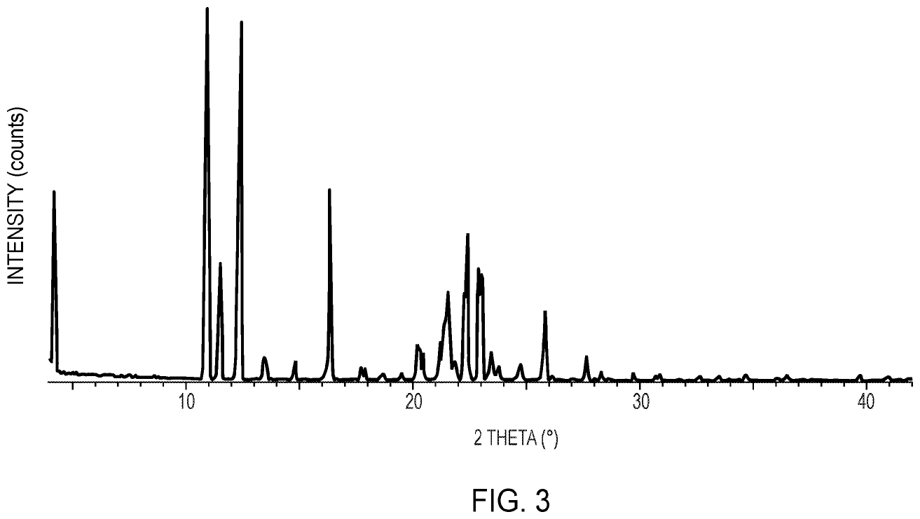

In yet other embodiments, the compound in crystalline polymorphic Form B is characterized by an X-ray powder diffraction pattern substantially the same as shown in FIG. 3.

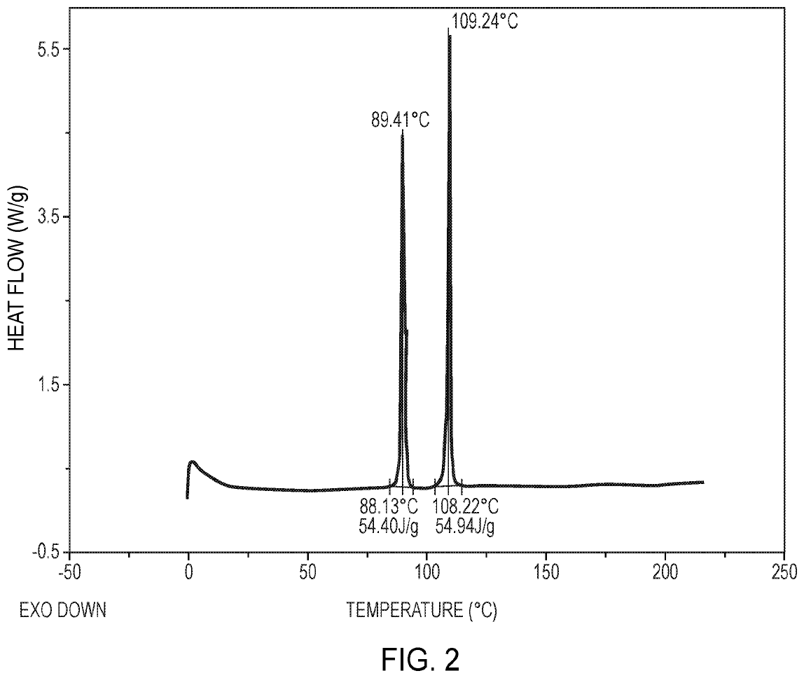

The compound in crystalline polymorphic Form B may also be characterized according to the temperature of melting point onset. Accordingly, in certain embodiments, the compound has a melting point onset as determined by differential scanning calorimetry in the range of from about 106 degrees Celsius to about 110 degrees Celsius, for example at about 108 degrees Celsius. In certain other embodiments, the compound has a differential scanning calorimetry curve substantially the same as shown in FIG. 2.

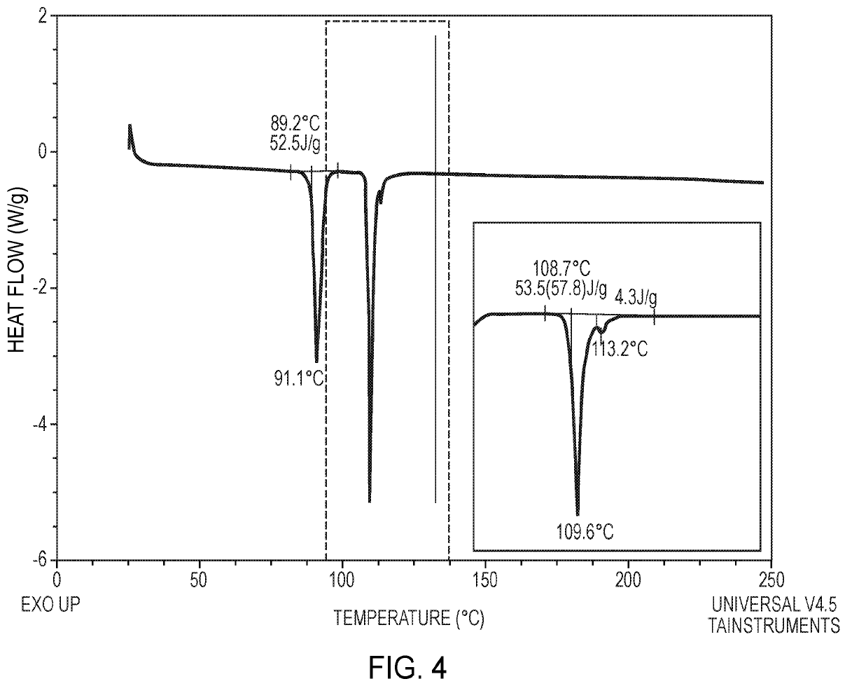

In certain other embodiments, the compound shows three endothermic events, as determined by differential scanning calorimetry, in the range of from about 89 degrees Celsius to about 115 degrees Celsius. In certain other embodiments, the compound exhibits a first endothermic event at about 91 degrees Celsius, a second at about 110 degrees Celsius and a third at about 113 degrees Celsius, as determined by differential scanning calorimetry. In certain other embodiments, the compound has a differential scanning calorimetry curve substantially the same as shown in FIG. 4.

### C. Crystalline Polymorphic Form C

In certain embodiments, the invention provides a compound in crystalline polymorphic Form C having the following formula:

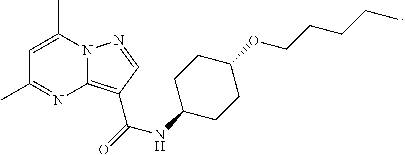 [SMILES: CCCCCO[C@H]1CC[C@H](NC(=O)c2cnn3c(C)cc(C)nc23)CC1]

In certain embodiments, the compound in crystalline form may be characterized by an X-ray powder diffraction pattern comprising peaks at the following diffraction angles (2θ): 4.9±0.2, 7.1±0.2, 9.9±0.2, and 12.4±0.2. In certain embodiments, the compound in crystalline form may be characterized by an X-ray powder diffraction pattern comprising peaks at the following diffraction angles (2θ): 4.9±0.2, 7.1±0.2, 9.9±0.2, 12.4±0.2, 14.9±0.2, 15.1±0.2, 19.9±0.2, 20.4±0.2, and 26.4±0.2.

In certain embodiments, the compound in crystalline polymorphic Form C is characterized by the X-ray powder diffraction pattern expressed in terms of diffraction angle 2θ and optionally relative intensity (expressed as a percentage with respect to the most intense peak) as set forth in Table 7.

TABLE 7
X-RAY POWDER DIFFRACTOGRAM DATA OF CRYSTALLINE
POLYMORPHIC FORM C.

| Angle (2θ°) | Intensity % |
|---|---|
| 4.9 | 100 |
| 7.1 | 16.2 |
| 9.9 | 46.2 |
| 10.2 | 7.1 |
| 11.0 | 5.4 |
| 11.3 | 10.2 |
| 12.4 | 10.6 |
| 13.9 | 4.1 |
| 14.1 | 3.9 |
| 14.5 | 3.4 |
| 14.9 | 10.6 |
| 15.1 | 10.8 |
| 15.7 | 4.2 |
| 16.0 | 4.7 |
| 16.6 | 8.0 |
| 18.0 | 8.0 |
| 19.3 | 9.2 |
| 19.9 | 11.2 |
| 20.4 | 12.9 |
| 21.0 | 4.2 |
| 26.0 | 5.6 |
| 26.4 | 17 |
| 27.4 | 6.0 |

In certain embodiments, the relative intensity of the peak at said diffraction angles (2θ) is at least 20% with respect to the most intense peak in the X-ray powder diffraction pattern.

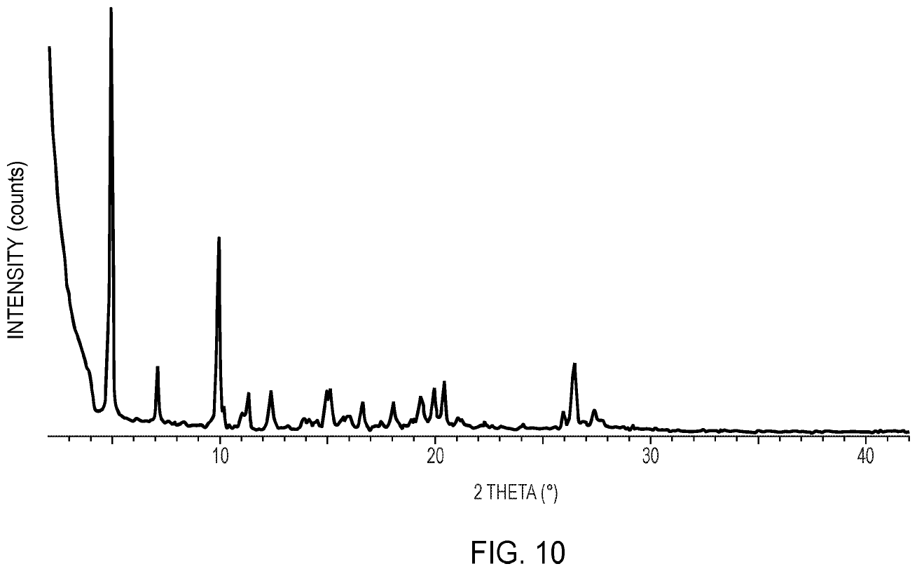

In yet other embodiments, the compound in crystalline polymorphic Form C is characterized by an X-ray powder diffraction pattern substantially the same as shown in FIG. 10.

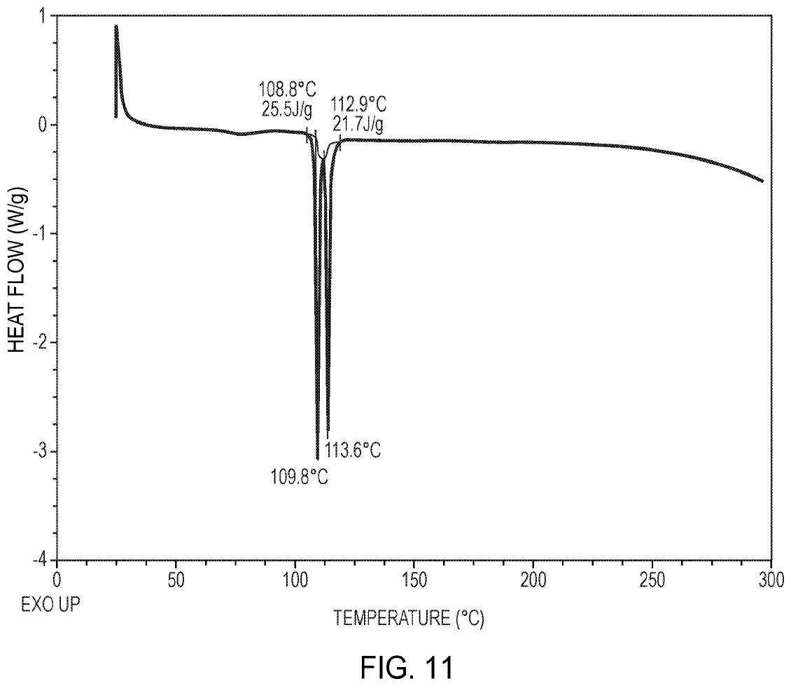

The compound in crystalline polymorphic Form C may also be characterized according to the temperature of melting point onset. Accordingly, in certain embodiments, the compound exhibits two endothermic events, as determined by differential scanning calorimetry, in the range of from about 108 degrees Celsius to about 116 degrees Celsius. In certain other embodiments, the compound exhibits a first endothermic event at about 110 degrees Celsius and a second at about 114 degrees Celsius, as determined by differential scanning calorimetry. In certain other embodiments, the compound has a melting point onset, as determined by differential scanning calorimetry, in the range of from about 108 degrees Celsius to about 114 degrees Celsius. In certain other embodiments, the compound has a phase transition onset and a melting point onset, as determined by differential scanning calorimetry, at about 109 degrees Celsius and 113 degrees Celsius, respectively. In certain other embodiments, the compound has a differential scanning calorimetry curve substantially the same as shown in FIG. 11.

### D. Crystalline Hydrate Form D

In certain embodiments, the invention provides a compound in crystalline hydrate Form D having the following formula:

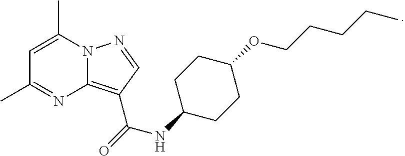 [SMILES: CCCCCO[C@H]1CC[C@H](NC(=O)c2cnn3c(C)cc(C)nc23)CC1]

In certain embodiments, the compound in crystalline form may be characterized by an X-ray powder diffraction pattern comprising peaks at the following diffraction angles (2θ): 3.8±0.2, 7.6±0.2, 9.4±0.2, and 14.1±0.2. In certain embodiments, the compound in crystalline form may be characterized by an X-ray powder diffraction pattern comprising peaks at the following diffraction angles (2θ): 3.8±0.2, 7.6±0.2, 9.4±0.2, 14.1±0.2, 22.1±0.2, 22.9±0.2, and 26.1±0.2.

In certain embodiments, the compound in crystalline hydrate Form D is characterized by the X-ray powder diffraction pattern expressed in terms of diffraction angle 2θ and optionally relative intensity (expressed as a percentage with respect to the most intense peak) as set forth in Table 8.

TABLE 8
X-RAY POWDER DIFFRACTOGRAM DATA OF CRYSTALLINE
HYDRATE FORM D.

| Angle (2θ°) | Intensity % |
|---|---|
| 3.8 | 100 |
| 7.3 | 3.5 |
| 7.6 | 35.7 |
| 9.1 | 3.1 |
| 9.4 | 23.1 |
| 10.3 | 4.4 |
| 11.0 | 6.5 |
| 11.5 | 2.6 |
| 12.5 | 1.5 |
| 13.0 | 5.3 |
| 14.0 | 7.6 |
| 14.1 | 11.5 |
| 14.4 | 2.4 |
| 14.6 | 2.7 |
| 15.0 | 3.4 |
| 15.3 | 3.7 |
| 15.6 | 7.6 |
| 16.7 | 4.2 |
| 18.2 | 7.3 |
| 18.9 | 5.6 |
| 19.1 | 7.0 |
| 19.9 | 5.3 |
| 20.1 | 7.0 |
| 20.5 | 2.9 |
| 21.8 | 4.8 |
| 22.1 | 17.3 |
| 22.9 | 11.5 |
| 23.6 | 5.5 |
| 24.3 | 3.9 |
| 24.8 | 4.8 |
| 25.4 | 8.6 |
| 26.1 | 11.2 |
| 27.9 | 3.8 |
| 28.1 | 4.1 |

In certain embodiments, the relative intensity of the peak at said diffraction angles (2θ) is at least 20% with respect to the most intense peak in the X-ray powder diffraction pattern.

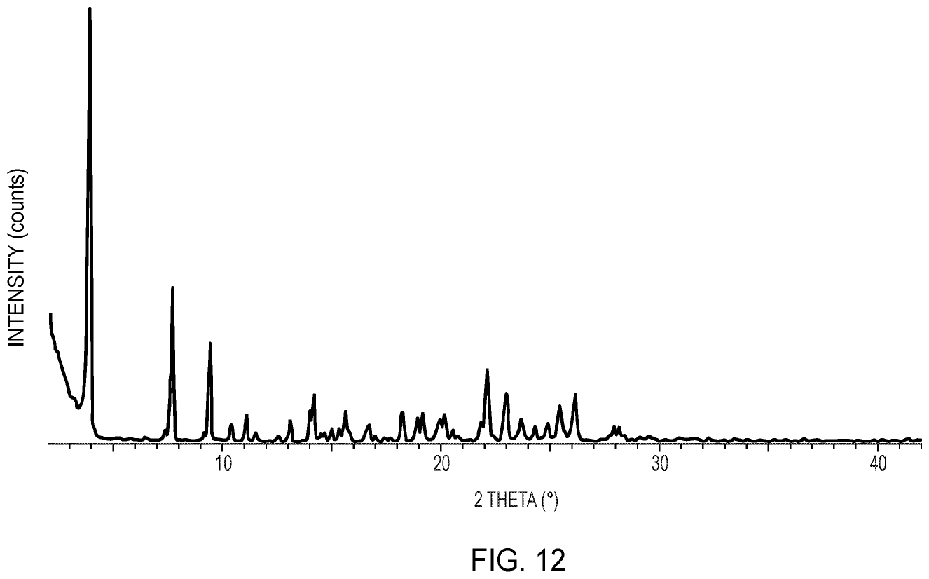

In yet other embodiments, the compound in crystalline hydrate Form D is characterized by an X-ray powder diffraction pattern substantially the same as shown in FIG. 12.

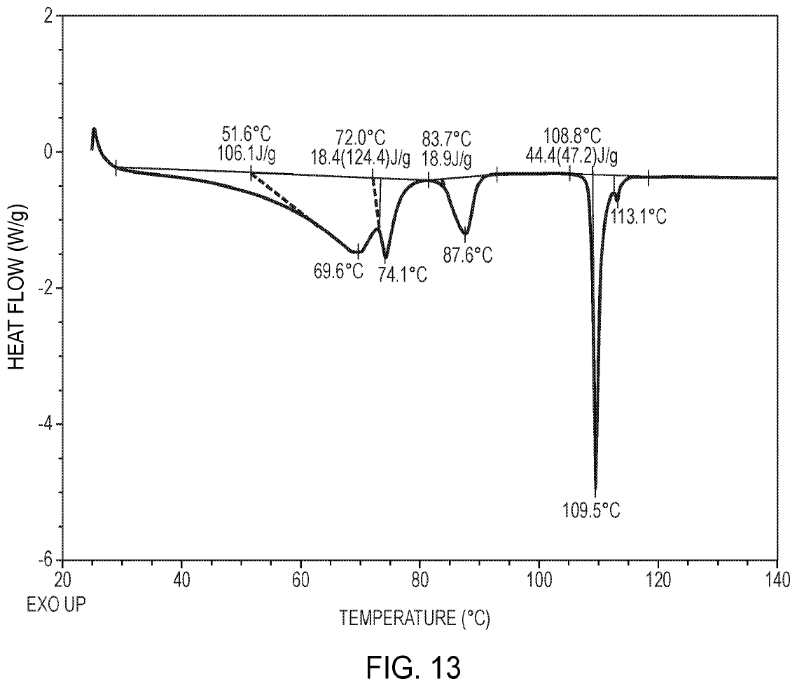

The compound in crystalline hydrate Form D may also be characterized according to the temperature of melting point onset. Accordingly, in certain embodiments, the compound exhibits one or more broad endothermic events in the range of from about 50 degrees Celsius to about 90 degrees Celsius and a sharp endothermic event in the range of from about 108 degrees Celsius to about 116 degrees Celsius, as determined by differential scanning calorimetry. In certain other embodiments, the compound exhibits a final endothermic event at about 110 degrees Celsius, as determined by differential scanning calorimetry. In certain other embodiments, the compound has a melting point onset, as determined by differential scanning calorimetry, in the range of from about 108 degrees Celsius to about 114 degrees Celsius. In certain other embodiments, the compound has a melting point onset, as determined by differential scanning calorimetry, at about 109 degrees Celsius. In certain other embodiments, the compound has a differential scanning calorimetry curve substantially the same as shown in FIG. 13.

### III. Therapeutic Applications

The invention provides methods of treating medical disorders, such as Gaucher disease, Parkinson's disease, Lewy body disease, dementia, multiple system atrophy, epilepsy, bipolar disorder, schizophrenia, an anxiety disorder, major depression, polycystic kidney disease, type 2 diabetes, open angle glaucoma, multiple sclerosis, endometriosis, and multiple myeloma, using a crystalline substituted cyclohexyl pyrazolo[1,5-a]pyrimidinyl carboxamide compound described herein, such as crystalline 5,7-dimethyl-N-((1S*,4S)-4-(pentyloxy)cyclohexyl)pyrazolo[1,5-a]pyrimidine-3-carboxamide. Treatment methods include the use of a crystalline substituted cyclohexyl pyrazolo[1,5-a]pyrimidinyl carboxamide compound described herein as a stand-alone therapeutic agent and/or as part of a combination therapy with another therapeutic agent. Although not wishing to be bound by a particular theory, it is understood that crystalline substituted cyclohexyl pyrazolo[1,5-a]pyrimidinyl carboxamide compounds described herein may activate glucocerebrosidase (GCase).

### A. Methods of Treating Medical Disorders

One aspect of the invention provides a method of treating disorder selected from the group consisting of Gaucher disease, Parkinson's disease, Lewy body disease, dementia, multiple system atrophy, epilepsy, bipolar disorder, schizophrenia, an anxiety disorder, major depression, polycystic kidney disease, type 2 diabetes, open angle glaucoma, multiple sclerosis, endometriosis, and multiple myeloma. The method comprises administering to a patient in need thereof a therapeutically effective amount of a crystalline substituted cyclohexyl pyrazolo[1,5-a]pyrimidinyl carboxamide compound described herein to treat the disorder. The compound may be crystalline 5,7-dimethyl-N-((1S*,4S)-4-(pentyloxy)cyclohexyl)pyrazolo[1,5-a]pyrimidine-3-carboxamide described above in Section II.

In certain embodiments, the compound is crystalline 5,7-dimethyl-N-((1S*,4S)-4-(pentyloxy)cyclohexyl)pyrazolo[1,5-a]pyrimidine-3-carboxamide polymorphic Form A. In certain embodiments, the compound is crystalline 5,7-dimethyl-N-((1S*,4S)-4-(pentyloxy)cyclohexyl)pyrazolo[1,5-a]pyrimidine-3-carboxamide polymorphic Form B. In certain embodiments, the compound is crystalline 5,7-dimethyl-N-((1S*,4S)-4-(pentyloxy)cyclohexyl)pyrazolo[1,5-a]pyrimidine-3-carboxamide polymorphic Form C. In certain embodiments, the compound is crystalline 5,7-dimethyl-N-((1S*,4S)-4-(pentyloxy)cyclohexyl)pyrazolo[1,5-a]pyrimidine-3-carboxamide hydrate Form D.

In certain embodiments, the disorder is Gaucher disease, Parkinson's disease, Lewy body disease, dementia, or multiple system atrophy. In certain embodiments, the disorder is Gaucher disease, Parkinson's disease, Lewy body disease, dementia, or multiple system atrophy. In certain other embodiments, the disorder is Gaucher disease. In certain embodiments, the disorder is Parkinson's disease. In certain embodiments, the disorder is Lewy body disease. In certain embodiments, the disorder is dementia. In certain embodiments, the disorder is a dementia selected from the group consisting of Alzheimer's disease, frontotemporal dementia, and a Lewy body variant of Alzheimer's disease. In certain embodiments, the disorder is multiple system atrophy.

In certain embodiments, the disorder is an anxiety disorder, such as panic disorder, social anxiety disorder, or generalized anxiety disorder.

Efficacy of the compounds in treating Gaucher disease, Parkinson's disease, Lewy body disease, dementia, multiple system atrophy, epilepsy, bipolar disorder, schizophrenia, an anxiety disorder, major depression, polycystic kidney disease, type 2 diabetes, open angle glaucoma, multiple sclerosis, endometriosis, and multiple myeloma may be evaluated by testing the compounds in assays known in the art for evaluating efficacy against these diseases and/or, e.g., for activation of glucocerebrosidase (GCase), as discussed in the Examples below.

In certain embodiments, the patient is a human.

The description above describes multiple embodiments relating to methods of treating various disorders using certain crystalline substituted cyclohexyl pyrazolo[1,5-a]pyrimidinyl carboxamide compounds. The patent application specifically contemplates all combinations of the embodiments. For example, the invention contemplates methods for treating Gaucher disease, Parkinson's disease, Lewy body disease, dementia, or multiple system atrophy by administering a therapeutically effective amount of crystalline 5,7-dimethyl-N-((1S*,4S)-4-(pentyloxy)cyclohexyl)pyrazolo[1,5-a]pyrimidine-3-carboxamide polymorphic Form A. Also, the invention contemplates methods for treating Gaucher disease, Parkinson's disease, Lewy body disease, dementia, or multiple system atrophy by administering a therapeutically effective amount of crystalline 5,7-dimethyl-N-((1S*,4S)-4-(pentyloxy)cyclohexyl)pyrazolo[1,5-a]pyrimidine-3-carboxamide polymorphic Form B.

### Medical Use and Preparation of Medicament

Another aspect of the invention relates to compounds and compositions described herein for use in treating a disorder described herein. Another aspect of the invention pertains to use of a compound or composition described herein in the preparation of a medicament for treating a disorder described herein.

### C. Combination Therapy

The invention embraces combination therapy, which includes the administration of a crystalline substituted cyclohexyl pyrazolo[1,5-a]pyrimidinyl carboxamide compound described herein and a second agent as part of a specific treatment regimen intended to provide the beneficial effect from the co-action of these therapeutic agents. The beneficial effect of the combination may include pharmacokinetic or pharmacodynamic co-action resulting from the combination of therapeutic agents.

Exemplary second agents for use in treating Gaucher disease include, for example, taliglucerase alfa, velaglucerase alfa, eliglustat, and miglustat. Exemplary second agents for use in treating Parkinson's disease include, for example, a glucosylceramide synthase inhibitor (e.g., ibiglustat), an acid ceramidase inhibitor (e.g., carmofur), an acid sphingomyelinase activator, levodopa, pramipexole, ropinirole, rotigotine, apomorphine, or salt thereof. Additional glucosylceramide synthase inhibitors for use in combination therapies include, for example, those described in International Patent Application Publications WO 2015/089067, WO 2014/151291, WO 2014/043068, WO 2008/150486, WO 2010/014554, WO 2012/129084, WO 2011/133915, and WO 2010/091164; U.S. Pat. Nos. 9,126,993, 8,961,959, 8,940,776, 8,729,075, and 8,309,593; and U.S. Patent Application Publications US 2014/0255381 and US 2014/0336174; each of which are hereby incorporated by reference. Additional acid ceramidase inhibitors for use in combination therapies include, for example, those described in International Patent Application Publications WO 2015/173168 and WO 2015/173169, each of which are hereby incorporated by reference.

### IV. Pharmaceutical Compositions

The invention provides pharmaceutical compositions comprising a crystalline substituted cyclohexyl pyrazolo[1,5-a]pyrimidinyl carboxamide compound described herein, such as crystalline 5,7-dimethyl-N-((1S*,4S)-4-(pentyloxy)cyclohexyl)pyrazolo[1,5-a]pyrimidine-3-carboxamide. In certain embodiments, the pharmaceutical compositions preferably comprise a therapeutically-effective amount of crystalline substituted cyclohexyl pyrazolo[1,5-a]pyrimidinyl carboxamide compound described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

In certain embodiments, the invention provides a pharmaceutical composition comprising a pharmaceutically acceptable carrier and a compound in crystalline form having the following formula

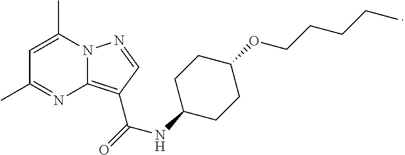 [SMILES: CCCCCO[C@H]1CC[C@H](NC(=O)c2cnn3c(C)cc(C)nc23)CC1]

In certain embodiments, the compound in crystalline form exhibits an X-ray powder diffraction pattern comprising peaks at the following diffraction angles (2θ): 5.7±0.2, 11.5±0.2, 11.8±0.2, and 12.8±0.2. In certain embodiments, the compound in crystalline form exhibits an X-ray powder diffraction pattern comprising peaks at the following diffraction angles (2θ): 5.7±0.2, 11.5±0.2, 11.8±0.2, 12.8±0.2, 17.2±0.2, 18.7±0.2, 19.6±0.2, 22.3±0.2, and 27.3±0.2. In certain embodiments, the compound in crystalline form exhibits an X-ray powder diffraction pattern comprising peaks at the following diffraction angles (2θ): 5.7±0.2, 12.8±0.2, 14.4±0.2, and 17.1±0.2. In certain embodiments, the compound in crystalline form exhibits an X-ray powder diffraction pattern comprising peaks at the following diffraction angles (2θ): 5.7±0.2, 12.8±0.2, 14.4±0.2, 17.1±0.2, 22.3±0.2, 23.0±0.2 and 27.2±0.2.

In certain embodiments, the invention provides a pharmaceutical composition comprising a pharmaceutically acceptable carrier and a compound in crystalline form having the following formula

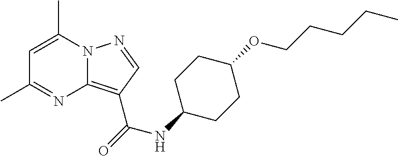 [SMILES: CCCCCO[C@H]1CC[C@H](NC(=O)c2cnn3c(C)cc(C)nc23)CC1]

that exhibits the X-ray powder diffraction pattern expressed in terms of diffraction angle 2θ, and optionally inter-planar distances d, and relative intensity (expressed as a percentage with respect to the most intense peak) as set forth in Table 1. In certain embodiments, the pharmaceutical composition is further characterized by the feature that the relative intensity of the peak at said diffraction angles (2θ) is at least 20% with respect to the most intense peak in the X-ray powder diffraction pattern.

In yet other embodiments, the invention provides a pharmaceutical composition comprising a pharmaceutically acceptable carrier and a compound in crystalline form having the following formula

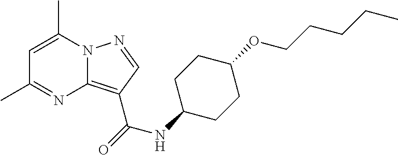 [SMILES: CCCCCO[C@H]1CC[C@H](NC(=O)c2cnn3c(C)cc(C)nc23)CC1]

that is characterized by an X-ray powder diffraction pattern substantially the same as shown in FIG. 5.

In certain embodiments, the invention provides a pharmaceutical composition comprising a pharmaceutically acceptable carrier and a compound in crystalline form having the following formula

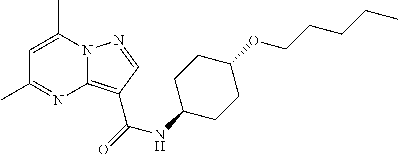 [SMILES: CCCCCO[C@H]1CC[C@H](NC(=O)c2cnn3c(C)cc(C)nc23)CC1]

that exhibits the X-ray powder diffraction pattern expressed in terms of diffraction angle 2θ and optionally relative intensity (expressed as a percentage with respect to the most intense peak) as set forth in Table 2. In certain embodiments, the pharmaceutical composition is further characterized by the feature that the relative intensity of the peak at said diffraction angles (2θ) is at least 20% with respect to the most intense peak in the X-ray powder diffraction pattern.

In yet other embodiments, the invention provides a pharmaceutical composition comprising a pharmaceutically acceptable carrier and a compound in crystalline form having the following formula

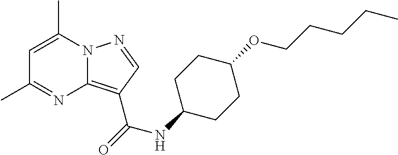 [SMILES: CCCCCO[C@H]1CC[C@H](NC(=O)c2cnn3c(C)cc(C)nc23)CC1]

wherein the crystalline polymorphic form exists in a monoclinic crystal system and has a P2₁/c space group and is optionally further characterized by the crystallographic unit cell parameters as set forth in Table 3.

In yet other embodiments, the invention provides a pharmaceutical composition comprising a pharmaceutically acceptable carrier and a compound in crystalline form having the following formula

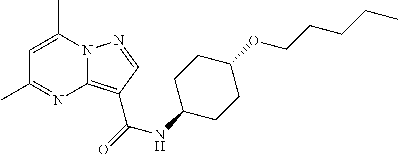 [SMILES: CCCCCO[C@H]1CC[C@H](NC(=O)c2cnn3c(C)cc(C)nc23)CC1]

that is characterized by an X-ray powder diffraction pattern substantially the same as shown in FIG. 7.

In certain embodiments, the invention provides a pharmaceutical composition comprising a pharmaceutically acceptable carrier and a compound in crystalline form having the following formula

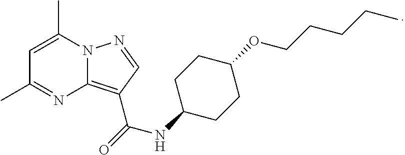 [SMILES: CCCCCO[C@H]1CC[C@H](NC(=O)c2cnn3c(C)cc(C)nc23)CC1]

In certain embodiments, the compound in crystalline form exhibits an X-ray powder diffraction pattern comprising peaks at the following diffraction angles (2θ): 4.0±0.2, 10.9±0.2, 12.3±0.2, and 16.2±0.2. In certain embodiments, the compound in crystalline form exhibits an X-ray powder diffraction pattern comprising peaks at the following diffraction angles (2θ): 4.0±0.2, 10.9±0.2, 12.3±0.2, 16.2±0.2, 20.2±0.2, 21.1±0.2, 21.5±0.2, 24.7±0.2, 27.6±0.2. In certain embodiments, the compound in crystalline form exhibits an X-ray powder diffraction pattern comprising peaks at the following diffraction angles (2θ): 4.2±0.2, 10.9±0.2, 11.5±0.2, and 12.4±0.2. In certain embodiments, the compound in crystalline form exhibits an X-ray powder diffraction pattern comprising peaks at the following diffraction angles (2θ): 4.2±0.2, 10.9±0.2, 11.5±0.2, 12.4±0.2, 16.3±0.2, 21.5±0.2, 22.3±0.2, 22.4±0.2, 22.9±0.2 and 23.0±0.2.

In certain embodiments, the invention provides a pharmaceutical composition comprising a pharmaceutically acceptable carrier and a compound in crystalline form having the following formula

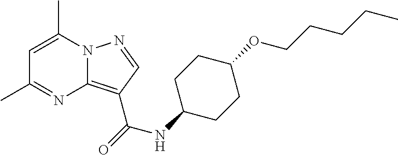 [SMILES: CCCCCO[C@H]1CC[C@H](NC(=O)c2cnn3c(C)cc(C)nc23)CC1]

that exhibits the X-ray powder diffraction pattern expressed in terms of diffraction angle 2θ, and optionally inter-planar distances d, and relative intensity (expressed as a percentage with respect to the most intense peak) as set forth in Table 4. In certain embodiments, the pharmaceutical composition is further characterized by the feature that the relative intensity of the peak at said diffraction angles (2θ) is at least 20% with respect to the most intense peak in the X-ray powder diffraction pattern.

In yet other embodiments, the invention provides a pharmaceutical composition comprising a pharmaceutically acceptable carrier and a compound in crystalline form having the following formula

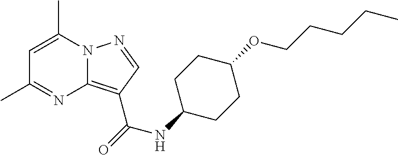 [SMILES: CCCCCO[C@H]1CC[C@H](NC(=O)c2cnn3c(C)cc(C)nc23)CC1]

that is characterized by an X-ray powder diffraction pattern substantially the same as shown in FIG. 1.

In certain embodiments, the invention provides a pharmaceutical composition comprising a pharmaceutically acceptable carrier and a compound in crystalline form having the following formula

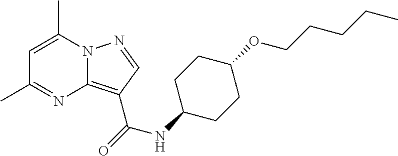 [SMILES: CCCCCO[C@H]1CC[C@H](NC(=O)c2cnn3c(C)cc(C)nc23)CC1]

that exhibits the X-ray powder diffraction pattern expressed in terms of diffraction angle 2θ and optionally relative intensity (expressed as a percentage with respect to the most intense peak) as set forth in Table 5. In certain embodiments, the pharmaceutical composition is further characterized by the feature that the relative intensity of the peak at said diffraction angles (2θ) is at least 20% with respect to the most intense peak in the X-ray powder diffraction pattern.

In certain embodiments, the invention provides a pharmaceutical composition comprising a pharmaceutically acceptable carrier and a compound in crystalline form having the following formula

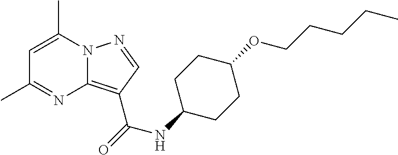 [SMILES: CCCCCO[C@H]1CC[C@H](NC(=O)c2cnn3c(C)cc(C)nc23)CC1]

wherein the crystalline polymorphic form exists in a monoclinic crystal system and has a P2₁/c space group and is optionally further characterized by the crystallographic unit cell parameters as set forth in Table 6.

In yet other embodiments, the invention provides a pharmaceutical composition comprising a pharmaceutically acceptable carrier and a compound in crystalline form having the following formula

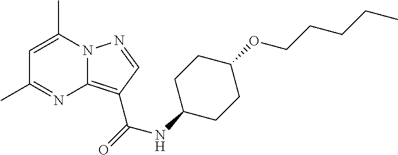 [SMILES: CCCCCO[C@H]1CC[C@H](NC(=O)c2cnn3c(C)cc(C)nc23)CC1]

that is characterized by an X-ray powder diffraction pattern substantially the same as shown in FIG. 3.

In certain embodiments, the invention provides a pharmaceutical composition comprising a pharmaceutically acceptable carrier and a compound in crystalline form having the following formula

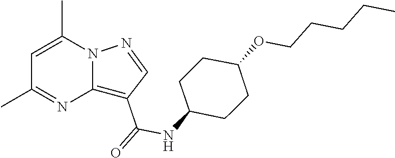 [SMILES: CCCCCO[C@H]1CC[C@H](NC(=O)c2cnn3c(C)cc(C)nc23)CC1]

that exhibits an X-ray powder diffraction pattern comprising peaks at the following diffraction angles (2θ): 4.9±0.2, 7.1±0.2, 9.9±0.2, 12.4±0.2, 14.9±0.2, 15.1±0.2, 19.9±0.2, 20.4±0.2, and 26.4±0.2. In certain embodiments, the invention provides a pharmaceutical composition comprising a pharmaceutically acceptable carrier and a compound in crystalline form having the following formula

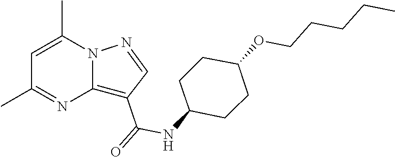 [SMILES: CCCCCO[C@H]1CC[C@H](NC(=O)c2cnn3c(C)cc(C)nc23)CC1]

that exhibits an X-ray powder diffraction pattern comprising peaks at the following diffraction angles (2θ): 4.9±0.2, 7.1±0.2, 9.9±0.2, and 12.4±0.2.

In certain embodiments, the invention provides a pharmaceutical composition comprising a pharmaceutically acceptable carrier and a compound in crystalline form having the following formula

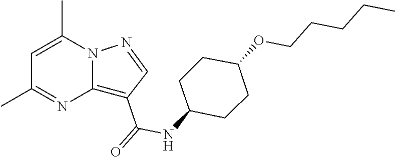 [SMILES: CCCCCO[C@H]1CC[C@H](NC(=O)c2cnn3c(C)cc(C)nc23)CC1]

that exhibits the X-ray powder diffraction pattern expressed in terms of diffraction angle 2θ and optionally relative intensity (expressed as a percentage with respect to the most intense peak) as set forth in Table 7. In certain embodiments, the pharmaceutical composition is further characterized by the feature that the relative intensity of the peak at said diffraction angles (2θ) is at least 20% with respect to the most intense peak in the X-ray powder diffraction pattern.

In yet other embodiments, the invention provides a pharmaceutical composition comprising a pharmaceutically acceptable carrier and a compound in crystalline form having the following formula

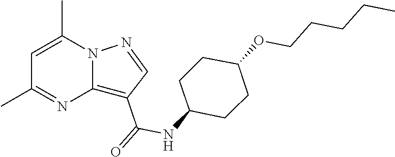 [SMILES: CCCCCO[C@H]1CC[C@H](NC(=O)c2cnn3c(C)cc(C)nc23)CC1]

that is characterized by an X-ray powder diffraction pattern substantially the same as shown in FIG. 10.

In certain embodiments, the invention provides a pharmaceutical composition comprising a pharmaceutically acceptable carrier and a compound in crystalline form having the following formula

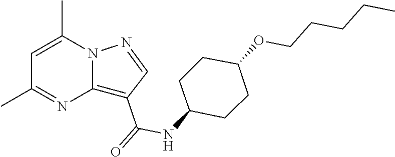 [SMILES: CCCCCO[C@H]1CC[C@H](NC(=O)c2cnn3c(C)cc(C)nc23)CC1]

that exhibits an X-ray powder diffraction pattern comprising peaks at the following diffraction angles (2θ): 3.8±0.2, 7.6±0.2, 9.4±0.2, 14.1±0.2, 22.1±0.2, 22.9±0.2, and 26.1±0.2. In certain embodiments, the invention provides a pharmaceutical composition comprising a pharmaceutically acceptable carrier and a compound in crystalline form having the following formula

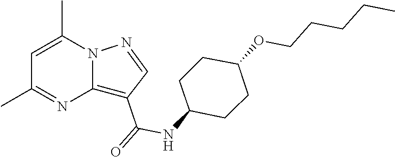 [SMILES: CCCCCO[C@H]1CC[C@H](NC(=O)c2cnn3c(C)cc(C)nc23)CC1]

that exhibits an X-ray powder diffraction pattern comprising peaks at the following diffraction angles (2θ): 3.8±0.2, 7.6±0.2, 9.4±0.2, and 14.1±0.2. In certain embodiments, the pharmaceutical composition is further characterized by the feature that the relative intensity of the peak at said diffraction angles (2θ) is at least 20% with respect to the most intense peak in the X-ray powder diffraction pattern.

In certain embodiments, the invention provides a pharmaceutical composition comprising a pharmaceutically acceptable carrier and a compound in crystalline form having the following formula

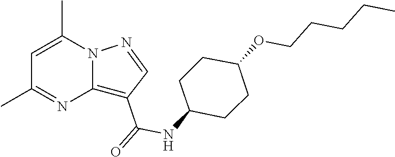 [SMILES: CCCCCO[C@H]1CC[C@H](NC(=O)c2cnn3c(C)cc(C)nc23)CC1]

that exhibits the X-ray powder diffraction pattern expressed in terms of diffraction angle 2θ and optionally relative intensity (expressed as a percentage with respect to the most intense peak) as set forth in Table 8.

In yet other embodiments, the invention provides a pharmaceutical composition comprising a pharmaceutically acceptable carrier and a compound in crystalline form having the following formula

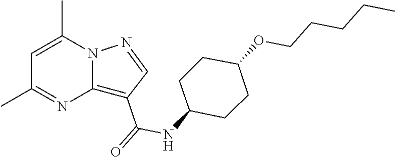 [SMILES: CCCCCO[C@H]1CC[C@H](NC(=O)c2cnn3c(C)cc(C)nc23)CC1]

that is characterized by an X-ray powder diffraction pattern substantially the same as shown in FIG. 12. In certain embodiments, the pharmaceutical composition is further characterized by the feature that the relative intensity of the peak at said diffraction angles (2θ) is at least 20% with respect to the most intense peak in the X-ray powder diffraction pattern.

The phrase “therapeutically-effective amount” as used herein means that amount of a compound, material, or composition comprising a compound of the present invention which is effective for producing some desired therapeutic effect in at least a sub-population of cells in a subject at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of a subject (e.g., a human being or an animal) without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration.

The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of a cyclodextrin, a cellulose, a liposome, a micelle forming agent, e.g., a bile acid, and a polymeric carrier, e.g., a polyester and a polyanhydride; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc.; administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

### V. Kits for Use in Medical Applications

Another aspect of the invention provides a kit for treating a disorder. The kit comprises: i) instructions for treating a medical disorder, such as Gaucher disease, Parkinson's disease, Lewy body disease, dementia, or multiple system atrophy; and ii) a crystalline substituted cyclohexyl pyrazolo[1,5-a]pyrimidinyl carboxamide compound described herein, such as crystalline 5,7-dimethyl-N-((1S*,4S)-4-(pentyloxy)cyclohexyl)pyrazolo[1,5-a]pyrimidine-3-carboxamide. The kit may comprise one or more unit dosage forms containing an amount of a crystalline substituted cyclohexyl pyrazolo[1,5-a]pyrimidinyl carboxamide compound described herein, such as crystalline 5,7-dimethyl-N-((1S*,4S)-4-(pentyloxy)cyclohexyl)pyrazolo[1,5-a]pyrimidine-3-carboxamide, that is effective for treating said medical disorder, e.g., Gaucher disease, Parkinson's disease, Lewy body disease, dementia, or multiple system atrophy.

### VI. Methods for Making Compounds

Another aspect of the invention provides methods for making compounds, including methods for making a compound in crystalline form having the following formula:

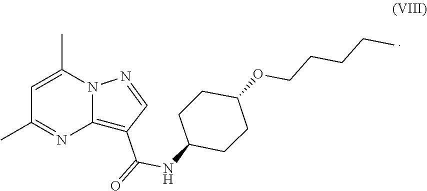 [SMILES: CCCCCO[C@H]1CC[C@H](NC(=O)c2cnn3c(C)cc(C)nc23)CC1]

In one approach the method comprises reacting a compound of Formula (IV):

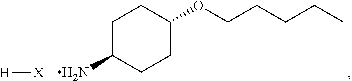

wherein X is an anion, with a compound of Formula (VII):

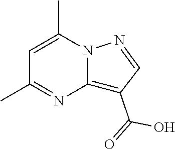

to produce a compound of Formula (VIII):

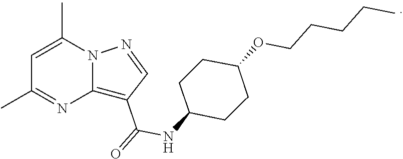 [SMILES: CCCCCO[C@H]1CC[C@H](NC(=O)c2cnn3c(C)cc(C)nc23)CC1]

### A. Synthesis of a Compound of Formula (IV)

The following sections describe two approaches for making the compound of formula (IV):

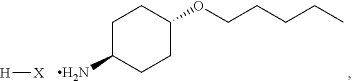

wherein X is an anion.

### I. Approach 1

In a first approach for making a compound of Formula (IV):

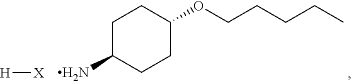

wherein X is an anion, the method comprises:
(a) admixing a compound of Formula (I), a base, and a solvent to produce a reaction mixture; wherein Formula (I) is represented by:

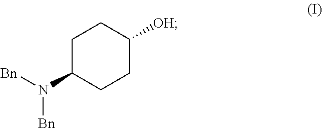 [SMILES: O[C@H]1CC[C@H](N(Cc2ccccc2)Cc2ccccc2)CC1]

(b) adding a n-pentyl alkylating agent (e.g., a n-pentyl halide, a n-pentyl sulfonate, or a n-pentyl phosphate) to the reaction mixture to produce a compound of Formula (II):

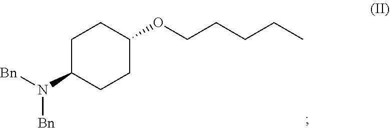 [SMILES: CCCCCO[C@H]1CC[C@H](N(Cc2ccccc2)Cc2ccccc2)CC1]

(c) exposing the compound of Formula (II) to acid HX to provide a compound of Formula (III):

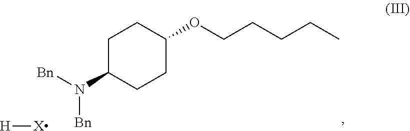

wherein X is an anion; and
(d) exposing the compound of Formula (III) to hydrogenation conditions, to provide a compound of Formula (IV):

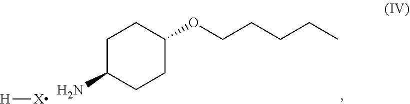

wherein X is an anion.

In certain embodiments, the compound of Formula (III) is a compound of Formula (III-a):

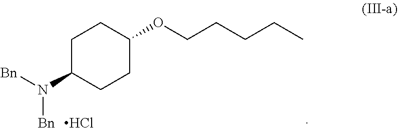 [SMILES: CCCCCO[C@H]1CC[C@H](N(Cc2ccccc2)Cc2ccccc2)CC1 ; Cl]

In certain embodiments, the compound of Formula (IV) is a compound of Formula (IV-a):

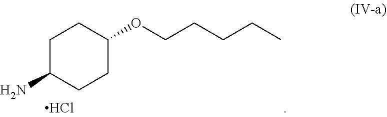

The above method may be further characterized by additional features, such as the base in step (a), the solvent in step (a), the acid in step (c), and the hydrogenation conditions in step (d), as discussed below.

More particularly, the method of making a compound of Formula (IV):

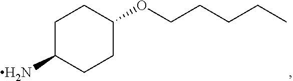

wherein X is an anion, comprises:
(a) admixing a compound of Formula (I), a base, and a solvent to produce a reaction mixture; wherein Formula (I) is represented by:

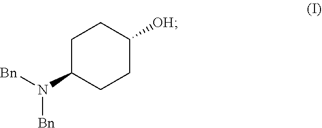 [SMILES: O[C@H]1CC[C@H](N(Cc2ccccc2)Cc2ccccc2)CC1]

(b) adding n-pentyl bromide to the reaction mixture to produce a compound of Formula (II):

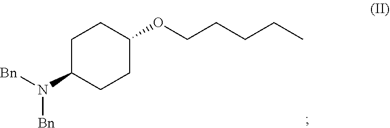 [SMILES: CCCCCO[C@H]1CC[C@H](N(Cc2ccccc2)Cc2ccccc2)CC1]

(c) exposing the compound of Formula (II) to acid HX to provide a compound of Formula (III):

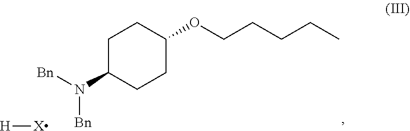

wherein X is an anion; and
(d) exposing the compound of Formula (III) to hydrogenation conditions, to provide a compound of Formula (IV):

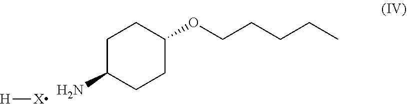

wherein X is an anion.

In certain embodiments, the compound of Formula (III) is a compound of Formula (III-a):

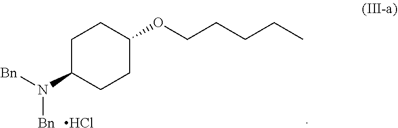 [SMILES: CCCCCO[C@H]1CC[C@H](N(Cc2ccccc2)Cc2ccccc2)CC1 ; Cl]

In certain embodiments, the compound of Formula (IV) is a compound of Formula (IV-a):

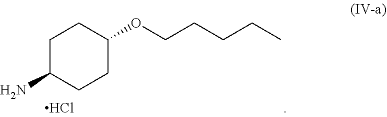

The above method may be further characterized by additional features, such as the base in step (a), the solvent in step (a), the acid in step (c), and the hydrogenation conditions in step (d), as discussed below.

a. Steps (a) and (b)

In certain embodiments, the base in step (a) is a metal hydride, a metal carbonate, a metal bicarbonate, or metal alkoxide (e.g., a metal butoxide). In certain embodiments, the base in step (a) is a metal hydride, a metal carbonate, or a metal bicarbonate. In certain embodiments, the base in step (a) is a metal hydride or metal alkoxide (e.g., a metal butoxide). In certain embodiments, the base in step (a) is a metal hydride. In certain embodiments, the base in step (a) is sodium hydride or potassium t-butoxide. In certain embodiments, the base in step (a) is sodium hydride.

In certain embodiments, the solvent in step (a) is a polar, aprotic organic solvent. In certain embodiments, the solvent in step (a) is dimethylacetamide, dimethylformamide, dimethylsulfoxide, diethyl ether, tetrahydrofuran, 1,4-dioxane, or a mixture thereof. In certain embodiments, the solvent in step (a) is dimethylacetamide, dimethylformamide, dimethylsulfoxide, diethyl ether, or 1,4-dioxane. In certain embodiments, the solvent in step (a) is dimethylacetamide or dimethylsulfoxide. In certain embodiments, the solvent in step (a) is dimethylsulfoxide, tetrahydrofuran, or a mixture thereof. In certain embodiments, the solvent in step (a) is a mixture of dimethylsulfoxide and tetrahydrofuran. In certain embodiments, the solvent in step (a) is dimethylsulfoxide. In certain embodiments, the solvent in step (a) is dimethylacetamide.

In certain embodiments, the temperature of the reaction mixture in step (a) is less than about 35 degrees Celsius. In certain embodiments, the temperature of the reaction mixture in step (b) is less than about 35 degrees Celsius. In certain embodiments, the temperature of the reaction mixture in steps (a) and (b) is independently less than about 35 degrees Celsius. In certain embodiments, the temperature of the reaction mixture in steps (a) and (b) is independently in the range of from about 0 degrees Celsius to about 35 degrees Celsius.

b. Step (c)

In certain embodiments, acid HX in step (c) is a mineral acid. In certain embodiments, acid HX in step (c) is hydrochloric acid, hydrobromic acid, sulfuric acid, nitric acid, or phosphoric acid. In certain embodiments, acid HX in step (c) is hydrochloric acid. In certain other embodiments, acid HX in step (c) is an organic carboxylic acid compound. In certain embodiments, acid HX in step (c) is acetic acid, trifluoroacetic acid, formic acid, benzoic acid, or nitrobenzoic acid. In certain other embodiments, acid HX in step (c) is an organic sulfonic acid compound. In certain embodiments, acid HX in step (c) is methanesulfonic acid, trifluoromethanesulfonic acid, benzenesulfonic acid, toluenesulfonic acid, or nitrobenzenesulfonic acid. In certain other embodiments, acid HX in step (c) is acetic acid, trifluoroacetic acid, formic acid, benzoic acid, nitrobenzoic acid, methanesulfonic acid, trifluoromethanesulfonic acid, benzenesulfonic acid, toluenesulfonic acid, or nitrobenzenesulfonic acid.

In certain embodiments, the step of exposing the compound of Formula II to acid HX in step (c) comprises adding to the compound of Formula II a solution containing acid HX and a (C₁₋₄ alkyl)-CO₂—(C₁₋₄ alkyl) solvent. In certain embodiments, the step of exposing the compound of Formula II to acid HX in step (c) comprises adding to the compound of Formula II a solution containing acid HX and ethyl acetate.

c. Step (d)

In certain embodiments, the hydrogenation conditions in step (d) comprise a hydrogenation catalyst and a hydrogen source. In certain embodiments, the hydrogenation catalyst is palladium hydroxide on carbon, palladium on carbon, or Raney nickel. In certain embodiments, the hydrogenation catalyst is palladium hydroxide on carbon. In certain embodiments, the hydrogenation catalyst is palladium on carbon. In certain embodiments, the hydrogen source is hydrogen gas, ammonium formate, or cyclohexene. In certain embodiments, the hydrogen source is hydrogen gas.

In certain embodiments, the hydrogenation conditions further comprise a solvent containing an alcohol, toluene, an ether (e.g., THF and MBTE), or mixtures thereof. In certain embodiments, the solvent is a saturated aliphatic alcohol. In certain embodiments, the solvent is methanol, ethanol, 1-propanol, 2-propanol, or a mixture thereof. In certain embodiments, the solvent is methanol. In certain embodiments, the solvent is ethanol.

In certain embodiments, the hydrogenation conditions are performed at (i) about atmospheric pressure or (ii) above atmospheric pressure (e.g., up to about 1 MPa). In certain embodiments, the hydrogenation conditions are performed at a temperature in the range of from about 20 degrees Celsius to about 60 degrees Celsius. In certain embodiments, the hydrogenation conditions are performed at a temperature in the range of from about 20 degrees Celsius to about 25 degrees Celsius. In certain embodiments, the hydrogenation is performed at about atmospheric pressure. In certain embodiments, the hydrogenation is performed at a hydrogen pressure of about 0.8 MPa. In certain embodiments, the hydrogenation conditions are performed at a hydrogen pressure of about 0.8 MPa at a temperature of about 50 degrees Celsius. In certain embodiments, the hydrogenation conditions are performed at about atmospheric pressure at a temperature in the range of from about 20 degrees Celsius to about 25 degrees Celsius.

### II. Approach 2

In a second approach for making a compound of Formula (IV):

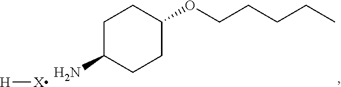

wherein X is an anion, the method comprises:
(a) admixing a compound of Formula (I), a base, and a solvent to produce a reaction mixture; wherein Formula (I) is represented by:

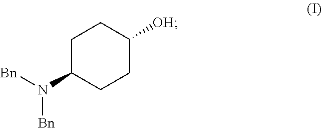 [SMILES: O[C@H]1CC[C@H](N(Cc2ccccc2)Cc2ccccc2)CC1]

(b) adding a n-pentyl alkylating agent (e.g., a n-pentyl halide, a n-pentyl sulfonate, or a n-pentyl phosphate) to the reaction mixture to produce a compound of Formula (II):

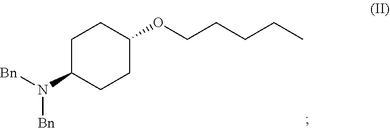 [SMILES: CCCCCO[C@H]1CC[C@H](N(Cc2ccccc2)Cc2ccccc2)CC1]

(c) exposing the compound of formula (II) to hydrogenation conditions to provide a compound of Formula (II-a):

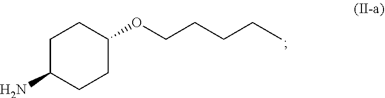

and
(d) exposing the compound of Formula (II-a) to acid HX to provide a compound of Formula (IV):

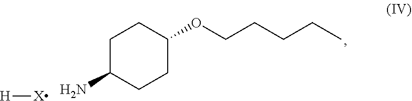

wherein X is an anion.

In certain embodiments, the compound of Formula (IV) is a compound of Formula (IV-a):

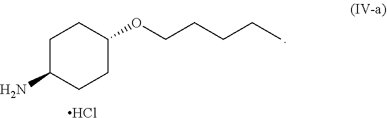

The above method may be further characterized by additional features, such as the base in step (a), the solvent in step (a), the hydrogenation conditions in step (c), and the acid in step (d) the hydrogenation conditions in step (d), as discussed below.

More particularly, the method of making a compound of Formula (IV):

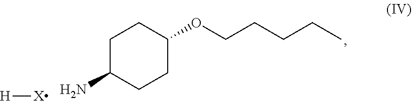

wherein X is an anion, comprises:
(a) admixing a compound of Formula (I), a base, and a solvent to produce a reaction mixture; wherein Formula (I) is represented by:

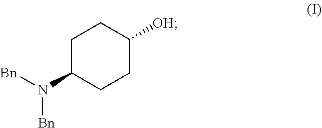 [SMILES: O[C@H]1CC[C@H](N(Cc2ccccc2)Cc2ccccc2)CC1]

(b) adding n-pentyl bromide to the reaction mixture to produce a compound of Formula (II):

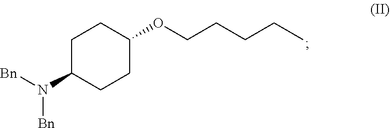 [SMILES: CCCCCO[C@H]1CC[C@H](N(Cc2ccccc2)Cc2ccccc2)CC1]

(c) exposing the compound of formula (II) to hydrogenation conditions to provide a compound of Formula (II-a):

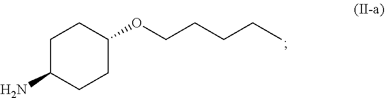

and
(d) exposing the compound of Formula (I-a) to acid HX to provide a compound of Formula (IV):

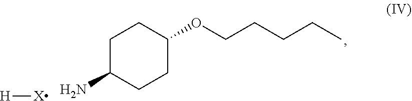

wherein X is an anion.

In certain embodiments, the compound of Formula (IV) is a compound of Formula (IV-a):

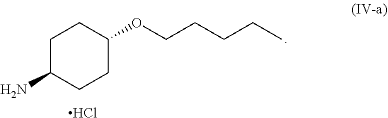

The above method may be further characterized by additional features, such as the base in step (a), the solvent in step (a), the hydrogenation conditions in step (c), and the acid in step (d) the hydrogenation conditions in step (d), as discussed below.

a. Steps (a) and (b)

In certain embodiments, the base in step (a) is a metal hydride, a metal carbonate, a metal bicarbonate, or metal alkoxide (e.g., a metal butoxide). In certain embodiments, the base in step (a) is a metal hydride, a metal carbonate, or a metal bicarbonate. In certain embodiments, the base in step (a) is a metal hydride or metal alkoxide (e.g., a metal butoxide). In certain embodiments, the base in step (a) is a metal hydride. In certain embodiments, the base in step (a) is sodium hydride or potassium t-butoxide. In certain embodiments, the base in step (a) is sodium hydride.

In certain embodiments, the solvent in step (a) is a polar, aprotic organic solvent. In certain embodiments, the solvent in step (a) is dimethylacetamide, dimethylformamide, dimethylsulfoxide, diethyl ether, tetrahydrofuran, 1,4-dioxane, or a mixture thereof. In certain embodiments, the solvent in step (a) is dimethylacetamide, dimethylformamide, dimethylsulfoxide, diethyl ether, or 1,4-dioxane. In certain embodiments, the solvent in step (a) is dimethylacetamide or dimethylsulfoxide. In certain embodiments, the solvent in step (a) is dimethylsulfoxide, tetrahydrofuran, or a mixture thereof. In certain embodiments, the solvent in step (a) is a mixture of dimethylsulfoxide and tetrahydrofuran. In certain embodiments, the solvent in step (a) is dimethylsulfoxide. In certain embodiments, the solvent in step (a) is dimethylacetamide.

In certain embodiments, the temperature of the reaction mixture in step (a) is less than about 35 degrees Celsius. In certain embodiments, the temperature of the reaction mixture in step (b) is less than about 35 degrees Celsius. In certain embodiments, the temperature of the reaction mixture in steps (a) and (b) is independently less than about 35 degrees Celsius. In certain embodiments, the temperature of the reaction mixture in steps (a) and (b) is independently in the range of from about 0 degrees Celsius to about 35 degrees Celsius.

b. Step (c)

In certain embodiments, the hydrogenation conditions in step (d) comprise a hydrogenation catalyst and a hydrogen source. In certain embodiments, the hydrogenation catalyst is palladium hydroxide on carbon, palladium on carbon, or Raney nickel. In certain embodiments, the hydrogenation catalyst is palladium hydroxide on carbon. In certain embodiments, the hydrogenation catalyst is palladium on carbon. In certain embodiments, the hydrogen source is hydrogen gas, ammonium formate, or cyclohexene. In certain embodiments, the hydrogen source is hydrogen gas.

In certain embodiments, the hydrogenation conditions further comprise a solvent containing an alcohol, toluene, an ether (e.g., THF and MBTE), or mixtures thereof. In certain embodiments, the solvent is a saturated aliphatic alcohol. In certain embodiments, the solvent is methanol, ethanol, 1-propanol, 2-propanol, or a mixture thereof. In certain embodiments, the solvent is methanol. In certain embodiments, the solvent is ethanol.

In certain embodiments, the hydrogenation conditions are performed at (i) about atmospheric pressure or (ii) above atmospheric pressure (e.g., up to about 1 MPa). In certain embodiments, the hydrogenation conditions are performed at a temperature in the range of from about 20 degrees Celsius to about 60 degrees Celsius. In certain embodiments, the hydrogenation conditions are performed at a temperature in the range of from about 20 degrees Celsius to about 25 degrees Celsius. In certain embodiments, the hydrogenation is performed at about atmospheric pressure. In certain embodiments, the hydrogenation is performed at a hydrogen pressure of about 0.8 MPa. In certain embodiments, the hydrogenation conditions are performed at a hydrogen pressure of about 0.8 MPa at a temperature of about 50 degrees Celsius. In certain embodiments, the hydrogenation conditions are performed at about atmospheric pressure at a temperature in the range of from about 20 degrees Celsius to about 25 degrees Celsius.

c. Step (d)

In certain embodiments, acid HX in step (c) is a mineral acid. In certain embodiments, acid HX in step (c) is hydrochloric acid, hydrobromic acid, sulfuric acid, nitric acid, or phosphoric acid. In certain embodiments, acid HX in step (c) is hydrochloric acid. In certain other embodiments, acid HX in step (c) is an organic carboxylic acid compound. In certain embodiments, acid HX in step (c) is acetic acid, trifluoroacetic acid, formic acid, benzoic acid, or nitrobenzoic acid. In certain other embodiments, acid HX in step (c) is an organic sulfonic acid compound. In certain embodiments, acid HX in step (c) is methanesulfonic acid, trifluoromethanesulfonic acid, benzenesulfonic acid, toluenesulfonic acid, or nitrobenzenesulfonic acid. In certain other embodiments, acid HX in step (c) is acetic acid, trifluoroacetic acid, formic acid, benzoic acid, nitrobenzoic acid, methanesulfonic acid, trifluoromethanesulfonic acid, benzenesulfonic acid, toluenesulfonic acid, or nitrobenzenesulfonic acid.

In certain embodiments, the step of exposing the compound of Formula II to acid HX in step (c) comprises adding to the compound of Formula II a solution containing acid HX and a (C₁₋₄ alkyl)-CO₂—(C₁₋₄ alkyl) solvent. In certain embodiments, the step of exposing the compound of Formula (II) to acid HX in step (c) comprises adding to the compound of Formula II a solution containing acid HX and ethyl acetate.

### B. Method of Producing a Compound of Formula (I) (the Starting Material for Making the Compound of Formula (IV))

This section describes the synthesis of a compound of Formula (I):

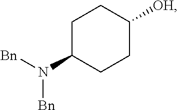 [SMILES: O[C@H]1CC[C@H](N(Cc2ccccc2)Cc2ccccc2)CC1]

which is the starting material used in Approaches 1 and 2 above for making the compound of Formula (IV):

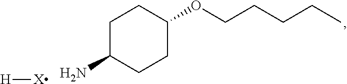

wherein X is an anion.

Briefly, a compound of Formula (V) is admixed with benzyl bromide in the presence of a base (B) and a solvent (S) to produce a compound of Formula (I), wherein Formula (V) is represented by:

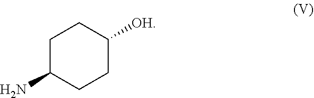

In certain embodiments, base (B) is potassium carbonate, potassium bicarbonate, sodium carbonate, sodium bicarbonate, cesium carbonate, or cesium bicarbonate. In certain embodiments, base (B) is potassium carbonate.

In certain embodiments, solvent (S) is a polar, aprotic organic solvent. In certain embodiments, solvent (S) is dimethylformamide, dimethylacetamide, dimethylsulfoxide, tetrahydrofuran, diethyl ether, or 1,4-dioxane. In certain embodiments, solvent (S) is dimethylformamide.

In certain embodiments, the step to produce a compound of Formula (I) is performed at a temperature less than about 35 degrees Celsius.

In certain other embodiments, the method further comprises admixing a compound of Formula (V) and substituted benzyl bromide in the presence of a base (B) and a solvent (S) to produce a compound of Formula (I) having Bn groups that contain at least one substituent, wherein the compound of Formula (V) is represented by:

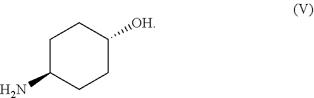

### C. Synthesis of a Compound of Formula (VII)

This section describes the synthesis of a compound of Formula (VII):

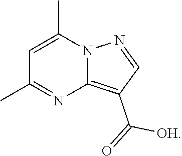

The method comprises admixing ethyl 3-amino-1H-pyrazole-4-carboxylate with pentane-2,4-dione in the presence of an acid and a solvent to produce a compound of Formula (VI), wherein the compound of Formula (VI) is represented by:

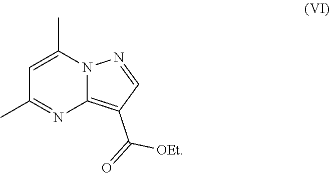 [SMILES: CCOC(=O)c1cnn2c(C)cc(C)nc12]

In certain embodiments, the acid is glacial acetic acid. In certain embodiments, the solvent is toluene.

In certain embodiments, the method further comprises admixing a compound of Formula (VI) with sodium hydroxide to produce a compound of Formula (VII), wherein the compound of Formula (VII) is represented by:

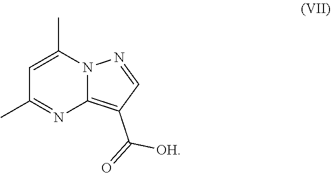

### D. Synthesis of a Compound of Formula (VIII)

The compound of Formula (VIII) can be produced by admixing a compound of Formula (VII) with an amide coupling reagent in the presence of a solvent (S1) to form an amide-coupling reaction mixture, and thereafter adding a compound of Formula (IV) to the amide-coupling reaction mixture, to produce a mixture containing a compound of Formula (VIII), wherein the compound of Formula (IV) is represented by

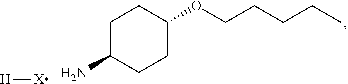

wherein X is an anion, the compound Formula (VII) is represented by

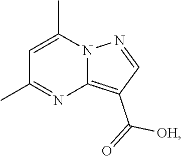

and Formula (VIII) is represented by:

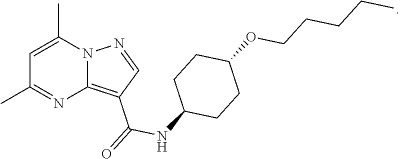 [SMILES: CCCCCO[C@H]1CC[C@H](NC(=O)c2cnn3c(C)cc(C)nc23)CC1]

In certain embodiments, the amide-coupling reagent comprises a uronium amide-coupling reagent, a phosphonium amide-coupling reagent, or a carbodiimide. In certain embodiments, the amide-coupling reagent comprises a uronium amide-coupling reagent. In certain embodiments, the amide-coupling reagent comprises O-(7-azabenzotriazol-1-yl)-N,N,N′,N′-tetramethyluronium hexafluorophosphate (HATU). In certain embodiments, the amide-coupling reagent comprises O-(benzotriazol-1-yl)-N,N,N′,N′-tetramethylaminium hexafluorophosphate (HBTU). In certain other embodiments, the amide-coupling reagent comprises a phosphonium amide-coupling reagent. In certain embodiments, the amide-coupling reagent comprises benzotriazol-1-yloxy-tris(dimethylamino)-phosphonium hexafluorophosphate (BOP), benzotriazol-1-yloxy-tripyrrolidino-phosphonium hexafluorophosphate (PyBOP), or bromo-tripyrrolidino-phosphonium hexafluorophosphate (PyBrOP). In certain other embodiments, the amide-coupling reagent comprises a carbodiimide. In certain embodiments, the amide-coupling reagent comprises N,N′-dicyclohexylcarbodiimide (DCC); N,N′-diisopropylcarbodiimide (DIC); or 1-ethyl-3-(3-dimethylaminopropyl)carbodiimide (EDC). In certain other embodiments, the amide-coupling reagent comprises 1-ethyl-3-(3-dimethylaminopropyl)carbodiimide (EDC). In certain other embodiments, the amide-coupling reagent comprises 2,4,6-tripropyl-1,3,5,2,4,6-trioxatriphosphorinane-2,4-6-trioxide (T3P). In certain other embodiments, the amide-coupling reagent comprises O-(7-azabenzotriazol-1-yl)-N,N,N′,N′-tetramethyluronium hexafluorophosphate (HATU) or 1-ethyl-3-(3-dimethylaminopropyl)carbodiimide (EDC). In certain other embodiments, an additive is added to the coupling reaction to accelerate the reaction. In certain embodiments the additive is 2-hydroxypyridine-N-oxide (HOPO). In certain other embodiments, the additive comprises 1-hydroxybenzotriazole (HOBt). In certain other embodiments, the additive comprises 1-hydroxy-7-azabenzotriazole (HOAt). In certain other embodiments, the additive comprises N-hydroxysuccinimide (HOSu).

In certain embodiments, the amide-coupling reagent further comprises a base. In certain embodiments, the amide-coupling reagent further comprises diisopropylethylamine (DIPEA), triethylamine, or N-methylmorpholine. In certain embodiments, the amide-coupling reagent further comprises diisopropylethylamine (DIPEA).

In certain embodiments, solvent (S1) is a polar, aprotic organic solvent. In certain embodiments, solvent (S1) comprises dimethylformamide, dimethylacetamide, or dimethylsulfoxide. In certain embodiments, solvent (S) comprises dimethylformamide.

In certain embodiments, the temperature of the amide-coupling reaction mixture is less than about 30 degrees Celsius.

### E. Methods of Making Crystalline Forms of the Compound of Formula (VIII)

It has been discovered that crystalline forms of the compound of Formula (VIII) can be produced by a variety of methods.

### I. Producing a Compound of Formula (VIII) as a Crystalline Solid by Precipitation from an Aqueous Mixture

In one approach for making a crystalline form of a compound of Formula (VIII) the method comprises adding water to the mixture containing a compound of Formula (VIII) produced in section C, to provide the compound of Formula (VIII) in the form of a crystalline solid. In certain embodiments, the volume of water added is in the range of about 0.5 to about 3 times the volume of the mixture containing a compound of Formula (VIII). In certain embodiments, the volume of water added is approximately equal to the volume of the mixture containing a compound of Formula (VIII).

In certain embodiments, the method further comprises the steps of:
(i) isolating the compound of Formula (VIII) in the form of a crystalline solid, to thereby provide an isolated crystalline compound of Formula (VIII); and
(ii) washing the isolated crystalline compound of Formula (VIII) one or more times with a solvent (S2) comprising water and dimethylformamide where the ratio of volume of water to dimethylformamide in solvent (S2) is in the range of 3:1 to 5:1, to provide a purified isolated crystalline compound of Formula (VIII).

In certain embodiments, the purified isolated crystalline compound of Formula (VIII) exhibits an X-ray powder diffraction pattern comprising peaks at the following diffraction angles (2θ): 4.0±0.2, 10.9±0.2, 12.3±0.2, 16.2±0.2, 20.2±0.2, 21.1±0.2, 21.5±0.2, 24.7±0.2, 27.6±0.2. In certain embodiments, the purified isolated crystalline compound of Formula (VIII) exhibits an X-ray powder diffraction pattern comprising peaks at the following diffraction angles (2θ): 4.2±0.2, 10.9±0.2, 11.5±0.2, 12.4±0.2, 16.3±0.2, 21.5±0.2, 22.3±0.2, 22.4±0.2, 22.9±0.2 and 23.0±0.2.

In certain embodiments, the purified isolated crystalline compound of Formula (VIII) exhibits an X-ray powder diffraction pattern comprising peaks at the following diffraction angles (2θ): 5.7±0.2, 11.5±0.2, 11.8±0.2, 12.8±0.2, 17.2±0.2, 18.7±0.2, 19.6±0.2, 22.3±0.2, and 27.3±0.2. In certain embodiments, the purified isolated crystalline compound of Formula (VIII) exhibits an X-ray powder diffraction pattern comprising peaks at the following diffraction angles (2θ): 5.7±0.2, 12.8±0.2, 14.4±0.2, 17.1±0.2, 22.3±0.2, 23.0±0.2 and 27.2±0.2.

In certain embodiments, the purified isolated crystalline compound of Formula (VIII) exhibits an X-ray powder diffraction pattern comprising peaks at the following diffraction angles (2θ): 3.8±0.2, 7.6±0.2, 9.4±0.2, 14.1±0.2, 22.1±0.2, 22.9±0.2, and 26.1±0.2.

### II. Producing a Compound of Formula (VIII) as a Crystalline Solid by Precipitation from a Mixture Containing an Alkane and an Ester Compound, or Other Solvent System

This section describes a variety of approaches for making crystalline polymorphic Forms A, B and C and crystalline hydrate Form D.

### (a) Crystalline Polymorphic Form A

The following protocol can be used to create crystalline polymorphic Form A. The method comprises the steps of:
(i) isolating the compound of Formula (VIII), produced in section C, in the form of a solid, to thereby provide an isolated compound of Formula (VIII);
(ii) admixing the isolated compound of Formula (VIII) with C₅₋₈ alkane and (C₁₋₄alkyl)-CO₂—(C₁₋₄ alkyl) to form a mixture, and then heating the mixture to a temperature of at least 65 degrees Celsius to provide a heated mixture;
(iii) cooling the heated mixture of step (ii) so that the temperature of the heated mixture is less than 55 degrees Celsius to provide a cooled mixture;
(iv) aging the cooled mixture of step (iii) to provide a compound of Formula (VIII) in the form of a crystalline solid; and
(v) isolating the compound of Formula (VIII) in the form of a crystalline solid to provide an isolated crystalline compound of Formula (VIII).

In certain embodiments, the C₅₋₈ alkane is heptane. In certain embodiments, the (C₁₋₄ alkyl)-CO₂—(C₁₋₄ alkyl) is ethyl acetate. In certain embodiments, the isolated crystalline compound of Formula (VIII) exhibits an X-ray powder diffraction pattern comprising peaks at the following diffraction angles (2θ): 5.7±0.2, 11.5±0.2, 11.8±0.2, 12.8±0.2, 17.2±0.2, 18.7±0.2, 19.6±0.2, 22.3±0.2, and 27.3±0.2.

### (b) Crystalline Polymorphic Form B

The following protocol can be used to create crystalline polymorphic Form B. The method comprises the steps of:
(i) isolating the compound of Formula (VIII), produced in section C, in the form of a solid, to thereby provide an isolated compound of Formula (VIII);
(ii) dissolving the isolated compound of Formula (VIII) in (C₁₋₄ alkyl)-CO₂—(C₁₋₄ alkyl) ester, or saturated aliphatic alcohol, or (C₁₋₄ alkyl)-CO—(C₁₋₄ alkyl) ketone solvent at a temperature in the range of about 20 degrees Celsius to about 50 degrees Celsius, thereby forming a mixture;
(iii) adding an alkane solvent to the mixture of step (ii) and allowing the mixture to cool to a temperature of from about 0 degrees Celsius to about 25 degrees Celsius;
(iv) aging the cooled mixture of step (iii) to provide a compound of Formula (VIII) in the form of a crystalline solid; and
(v) isolating the compound of Formula (VIII) in the form of a first crystalline solid to provide a first isolated crystalline compound of Formula (VIII), namely Form B.

In certain embodiments, the C₅₋₈ alkane is heptane. In certain embodiments, the C₅₋₈ alkane is methylcyclohexane. In certain embodiments, the (C₁₋₄ alkyl)-CO₂—(C₁₋₄ alkyl) is ethyl acetate. In certain embodiments, the (C₁₋₄ alkyl)-CO₂—(C₁₋₄ alkyl) is butyl acetate. In certain embodiments, the saturated alcohol is 1-pentanol. In certain embodiments, the saturated alcohol is isopentanol. In certain embodiments, the (C₁₋₄ alkyl)-CO—(C₁₋₄ alkyl) is methyl ethyl ketone. In certain embodiments, the (C₁₋₄ alkyl)-CO—(C₁₋₄ alkyl) is methyl isobutyl ketone (MIBK). In certain embodiments, the isolated crystalline compound of Formula (VIII) exhibits an X-ray powder diffraction pattern comprising peaks at the following diffraction angles (2θ): 4.2±0.2, 10.9±0.2, 11.5±0.2, 12.4±0.2, 16.3±0.2, 21.5±0.2, 22.3±0.2, 22.4±0.2, 22.9±0.2 and 23.0±0.2.

### (c) Crystalline Polymorphic Form A

The following protocol can be used to create crystalline polymorphic Form A from crystalline polymorphic Form B. The method comprises the steps of:
(i) dissolving the first isolated crystalline compound of Formula (VIII), namely Form B, in ethyl acetate at a temperature of about 40 degrees Celsius, thereby forming a mixture;
(ii) adding heptane to the mixture of step (i) and heating the mixture to a temperature of about 75 degrees Celsius;
(iii) cooling the mixture of step (ii) to a temperature of about 50 degrees Celsius and adding seeds of a second isolated crystalline compound of Formula (VIII), thereby producing a seeded mixture;
(iv) aging the seeded mixture of step (iii) to provide a compound of Formula (VIII) in the form of a second crystalline solid; and
(v) isolating the compound of Formula (VIII) in the form of a crystalline solid to provide the second isolated crystalline compound of Formula (VIII), namely Form A.

In certain embodiments, the second isolated crystalline compound of Formula (VIII) exhibits an X-ray powder diffraction pattern comprising peaks at the following diffraction angles (2θ): 5.7±0.2, 12.8±0.2, 14.4±0.2, 17.1±0.2, 22.3±0.2, 23.0±0.2 and 27.2±0.2.

### (d) Crystalline Polymorphic Form C

The following protocol can be used to create crystalline polymorphic Form C from crystalline polymorphic Form A. The method comprises the steps of
(i) dissolving the second isolated crystalline compound of Formula (VIII), namely Form A, in a water miscible solvent at a temperature of about 50 degrees Celsius, thereby forming a mixture;
(ii) adding water to the mixture of step (i); and
(iii) isolating the compound of Formula (VIII) in the form of a crystalline solid to provide a third isolated crystalline compound of Formula (VIII), namely Form C.

In certain embodiments, in step (i), the water miscible solvent is acetic acid. In certain embodiments, in step (i), the water miscible solvent is t-butanol.

In certain embodiments, step (iii) is performed using lyophilization.

In certain embodiments, the third isolated crystalline compound of Formula (VIII) exhibits an X-ray powder diffraction pattern comprising peaks at the following diffraction angles (2θ): 4.9±0.2, 7.1±0.2, 9.9±02, 12.4±0.2, 14.9±0.2, 15.1±0.2, 19.9±0.2, 20.4±0.2, and 26.4±0.2.

### (e) Crystalline Hydrate Form D

The following protocol can be used to create crystalline hydrate Form D from crystalline polymorphic Form B. The method comprises the steps of
(i) adding an isolated crystalline compound of Formula (VIII) to water, thereby forming a mixture;
(ii) aging the mixture of step (i); and
(iii) isolating the compound of Formula (VIII) in the form of a crystalline solid to provide a fourth isolated crystalline compound of Formula (VIII), namely Form D.

In certain embodiments, the isolated crystalline compound of Formula (VIII) is added to water at a temperature in the range of from about 20 degrees Celsius to about 40 degrees Celsius. In certain embodiments, the isolated crystalline compound of Formula (VIII) is added to water at a temperature of about 20 degrees Celsius, about 25 degrees Celsius, about 30 degrees Celsius, about 35 degrees Celsius, or 40 degrees Celsius. In certain embodiments, the isolated crystalline compound of Formula (VIII) is added to water at a temperature of about 25 degrees Celsius. In certain embodiments, the isolated crystalline compound of Formula (VIII) is the first isolated crystalline compound of Formula (VIII), namely Form B. In certain embodiments, the isolated crystalline compound of Formula (VIII) is the second isolated crystalline compound of Formula (VIII), namely Form A. In certain embodiments, the isolated crystalline compound of Formula (VIII) is the third isolated crystalline compound of Formula (VIII), namely Form C.

In certain embodiments, the fourth isolated crystalline compound of Formula (VIII) exhibits an X-ray powder diffraction pattern comprising peaks at the following diffraction angles (2θ): 3.8±0.2, 7.6±0.2, 9.4±0.2, 14.1±0.2, 22.1±0.2, 22.9±0.2, and 26.1±0.2.

The description above describes multiple aspects and embodiments of the invention, including crystalline substituted cyclohexyl pyrazolo[1,5-a]pyrimidinyl carboxamide compounds, compositions comprising a crystalline substituted cyclohexyl pyrazolo[1,5-a]pyrimidinyl carboxamide compound, methods for making the crystalline substituted cyclohexyl pyrazolo[1,5-a]pyrimidinyl carboxamide compounds, methods of using the crystalline substituted cyclohexyl pyrazolo[1,5-a]pyrimidinyl carboxamide compounds, and kits. The patent application specifically contemplates all combinations and permutations of the aspects and embodiments. For example, the invention contemplates treating Gaucher disease, Parkinson's disease, Lewy body disease, dementia, or multiple system atrophy in a human patient by administering a therapeutically effective amount of crystalline 5,7-dimethyl-N-((1S*,4S)-4-(pentyloxy)cyclohexyl)pyrazolo[1,5-a]pyrimidine-3-carboxamide polymorphic Form A. Further, for example, the invention contemplates a kit for treating Gaucher disease, Parkinson's disease, Lewy body disease, dementia, or multiple system atrophy, the kit comprising instructions for treating Gaucher disease, Parkinson's disease, Lewy body disease, dementia, or multiple system atrophy and ii) a crystalline substituted cyclohexyl pyrazolo[1,5-a]pyrimidinyl carboxamide compound described herein, such as crystalline 5,7-dimethyl-N-((1S*,4S)-4-(pentyloxy)cyclohexyl)pyrazolo[1,5-a]pyrimidine-3-carboxamide polymorphic Form A. Also, the invention contemplates treating Gaucher disease, Parkinson's disease, Lewy body disease, dementia, or multiple system atrophy in a human patient by administering a therapeutically effective amount of crystalline 5,7-dimethyl-N-((1S*,4S)-4-(pentyloxy)cyclohexyl)pyrazolo[1,5-a]pyrimidine-3-carboxamide polymorphic Form B. Further, for example, the invention contemplates a kit for treating Gaucher disease, Parkinson's disease, Lewy body disease, dementia, or multiple system atrophy, the kit comprising instructions for treating Gaucher disease, Parkinson's disease, Lewy body disease, dementia, or multiple system atrophy and ii) a crystalline substituted cyclohexyl pyrazolo[1,5-a]pyrimidinyl carboxamide compound described herein, such as crystalline 5,7-dimethyl-N-((1S*, 4S)-4-(pentyloxy)cyclohexyl)pyrazolo[1,5-a]pyrimidine-3-carboxamide polymorphic Form B.

### EXAMPLES

The invention now being generally described, will be more readily understood by reference to the following examples, which are included merely for purposes of illustration of certain aspects and embodiments of the present invention, and are not intended to limit the invention.

### Example 1—Preparation of trans-4-(pentyloxy)cyclohexanaminium Chloride (Compound of Formula (IV-A))

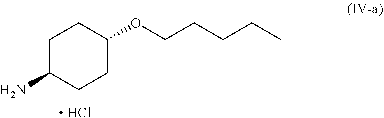

### Step 1—Preparation of trans-N,N-Dibenzyl-4-aminocyclohexanol (Compound of Formula (II))

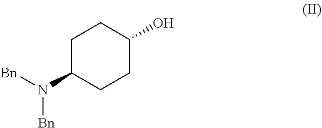 [SMILES: O[C@H]1CC[C@H](N(Cc2ccccc2)Cc2ccccc2)CC1]

To a slurry of 4.20 kg of trans-4-aminocyclohexanol (Compound of Formula (I), 36.5 mol) and 15.1 kg of potassium carbonate (109.4 mol; 3.0 equiv.) in 13.2 L of DMF cooled to 10° C. was added 13.7 kg of benzyl bromide (80.2 mol; 2.2 equiv.) over 1.5 h while keeping the temperature in the range of 25−30° C. The mixture was stirred at this temperature for another 3 hours before 800 mL of methanol was added. The mixture was stirred for another 30 min before 50 L of water and 32 L of MTBE were added. The layers were separated and the aqueous layer was extracted with another 21 L of MTBE. The combined organic extracts were dried over 8 kg of sodium sulfate, filtered, and the cake was washed with 12 L of MTBE. The MTBE solution was concentrated to a total volume of approximately 22 L under vacuum. Then, to the resulting slurry was added 26 L of petroleum ether, and then the petroleum ether was removed by vacuum. Next, the resulting slurry was diluted with another 26 L of petroleum ether, and then the petroleum ether was removed by vacuum. Next, to the resulting slurry, 60 L of petroleum ether was added. The resulting slurry was stirred for 1.5 hours at 20-25° C. and then filtered. The solids were washed twice with 24 L of petroleum ether and dried under vacuum at 50° C. for 5 hours. A total of 9.1 kg of the compound of Formula (I) was obtained in 82% yield (corrected for purity). ¹H NMR (400 MHz, CDCl₃) δ ppm 7.37-7.19 (m, 10H), 3.61 (s, 4H), 3.58-3.52 (m, 1H), 2.52-2.49 (m, 1H), 1.99 (d, J=11.5 Hz, 2H), 1.89 (d, J=12.5 Hz, 2H), 1.47-1.37 (m, 3H), 1.26-1.14 (m, 2H).

### Step 2—Preparation of trans-N,N-Dibenzyl-4-(pentyloxy)cyclohexanaminium chloride (Compound of Formula (III-a))

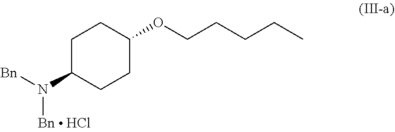 [SMILES: CCCCCO[C@H]1CC[C@H](N(Cc2ccccc2)Cc2ccccc2)CC1 ; Cl]

To a solution of 4.00 kg of trans-N,N-dibenzyl-4-aminocyclohexanol (Compound of Formula (II) from Step 1, 13.52 mol) in 32 L of DMA at 10° C. was added 2.16 kg of sodium hydride (60 wt %; 54.0 mol; 4.0 equiv.) portion wise under a nitrogen atmosphere. The resulting mixture was stirred at 20-25° C. for 30 min before 8.16 kg of pentyl bromide (54.1 mol; 4.0 equiv.) was added dropwise while keeping the temperature in the range of 25−35° C. The resulting mixture was stirred at 25-30° C. for 5 hours and then cooled down to 10-15° C., before 2.0 L of water was added slowly in order to keep the temperature below 15° C. After addition of another 2 L of water a clear solution was obtained. The solution was partitioned between 60 L of water and 30 L of MTBE and the layers were separated. The aqueous layer was extracted with 20 L of MTBE. The combined organic extracts were washed twice with 30 L portions of water. washed once with 10 L of a saturated sodium chloride solution, and then dried over 6 kg of sodium sulfate. The solids were filtered and washed with 10 L of MTBE. The combined filtrates were concentrated under vacuum to near dryness. To the resulting residue was added 15 L of a 1 M solution of HCl in EtOAc. The resulting slurry was stirred for 2 hours and then filtered. The solids were washed with 10 L of MTBE and dried under vacuum at 50° C. for 6 hours. A total of 5.1 kg of the compound of Formula (III-a) was obtained in 88% yield (corrected for purity). ¹H NMR (400 MHz, DMSO-d₆) δ ppm 10.67 (s, 1H), 7.60 (m, 4H), 7.41 (m, 6H), 4.44 (dd, J=13.2, 4.5 Hz, 2H), 4.13 (dd, J=13.3, 5.9 Hz, 2H), 3.36 (t, J=6.3 Hz, 2H), 3.17 (m, 1H), 3.03 (m, 1H), 2.24 (d, J=11.1 Hz, 2H), 2.07 (d, J=10.2 Hz, 2H), 1.79-1.65 (m, 2H), 1.47-1.39 (m, 2H), 1.25 (m, 4H), 1.03-0.92 (m, 2H), 0.85 (t, J=6.6 Hz, 3H).

### Step 3—Preparation of trans-4-(Pentyloxy)cyclohexanaminium chloride (Compound of Formula (IV-a))

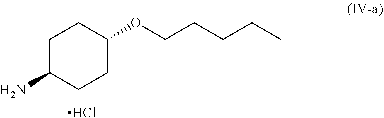

A slurry of 20.0 kg of trans-N,N-dibenzyl-4-(pentyloxy)cyclohexanaminium chloride (Compound of Formula III-a from Step 2, 94 wt % pure, 46.7 mol) and 2.0 kg of 20 wt % Pd(OH)₂/C in 100 L of methanol was hydrogenated at atmospheric pressure at room temperature for 20 hours and then filtered over 1 kg of Celite. The cake was washed with 7.5 L of methanol. The combined filtrates were concentrated to near dryness. To the residue was added 150 L of MTBE at 45-50° C. The resulting slurry was cooled at 10° C. over 3 hours, stirred for another hour, and then filtered. The solids were washed with 15 L of MTBE and dried under vacuum at 45° C. for 10 hours. A total of 8.3 kg of the compound of Formula (IV-a) was obtained in 80% yield (corrected for purity). ¹H NMR (400 MHz, DMSO-d₆) δ ppm 8.20 (br s, 3H), 3.37 (t, J=6.5 Hz, 2H), 3.14 (tt, J=10.4, 3.6 Hz, 1H), 2.93 (m, 1H), 2.00-1.90 (m, 4H), 1.50-1.12 (m, 10H), 0.86 (t, J=6.9 Hz, 3H).

### Example 2—Alternative Preparation of trans-N,N-Dibenzyl-4-(pentyloxy)cyclohexanaminium Chloride (Compound of Formula (III-a))

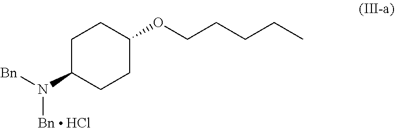 [SMILES: CCCCCO[C@H]1CC[C@H](N(Cc2ccccc2)Cc2ccccc2)CC1 ; Cl]

A solution of 100 g of trans-N,N-dibenzyl-4-aminocyclohexanol (Compound of Formula (I) from Example 1 (Step 1), 338 mmol) and 205 g of pentyl bromide (1.36 mol; 4.0 equiv.) in a mixture of 300 mL of dry DMSO and 300 mL of dry THF was cooled to a temperature in the range of 0° C. to 5° C. To the resulting mixture, a solution of 153 g of potassium tert-butoxide (1.35 mol; 4.0 equiv.) in a mixture of 300 mL of dry DMSO and 300 mL of dry THF was added slowly over 2 hrs maintaining the temperature of the reaction mixture below 5° C. The resulting mixture was stirred for another one hour before 400 mL of water was added while maintaining the temperature of the reaction mixture in the range of 0° C. to 5° C. After stirring the reaction mixture for another 15 min, the layers in the reaction mixture were allowed to settle. Then, the organic layer was separated and the aqueous layer was extracted with 200 mL of toluene. The combined organic layers were washed with 2×200 mL of 12% wt/wt of NaCl in water and 200 mL of a saturated NaCl solution. The resulting organic layer was then concentrated to a total volume of approximately 400 mL via distillation under reduced pressure to product a mixture. To this mixture was added 800 mL of toluene, the resulting solution was concentrated to a total volume of approximately 800 mL via distillation under reduced pressure to remove any residual THF and water. To the resulting solution was added 100 mL of a 1 M solution of HCl in EtOAc. The resulting slurry was stirred at room temperature for 2 h and then filtered. The solids were washed with 200 mL of toluene and then dried under vacuum at 50° C. for 6 hours. A total of 118 g of the title compound was obtained in 85% yield (corrected for purity). ¹H NMR (400 MHz, DMSO-d₆) δ ppm 10.67 (s, 1H), 7.60 (m, 4H), 7.41 (m, 6H), 4.44 (dd, J=13.2, 4.5 Hz, 2H), 4.13 (dd, J=13.3, 5.9 Hz, 2H), 3.36 (t, J=6.3 Hz, 2H), 3.17 (m, 1H), 3.03 (m, 1H), 2.24 (d, J=11.1 Hz, 2H), 2.07 (d, J=10.2 Hz, 2H), 1.79-1.65 (m, 2H), 1.47-1.39 (m, 2H), 1.25 (m, 4H), 1.03-0.92 (m, 2H), 0.85 (t, J=6.6 Hz, 3H).

### Example 3—Alternative Preparation of trans-4-(Pentyloxy)cyclohexanaminium Chloride (Compound of Formula (IV-A))

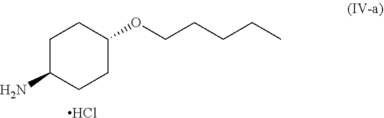

A mixture of 100 g of the compound of Formula (III-a) from Example 2 (245 mmol, corrected for purity) and 3 g of 10 wt % Pd/C catalyst in 500 mL of ethanol was hydrogenated at 1 MPa hydrogen pressure at a temperature of 50° C. for 8 hours. After cooling the reaction mixture to room temperature, the reaction mixture was filtered over Celite. The filter cake was washed with 100 mL of ethanol. The combined filtrates were concentrated to a total volume of approximately 200 mL via distillation under reduced pressure. Then, to the resulting mixture, 500 mL of toluene was added. The resulting mixture was then concentrated to a total volume of approximately 500 mL via distillation under reduced pressure before another 500 mL of toluene was added slowly while distilling at the same rate to keep the total volume constant. Upon completion of the distillation, the resulting slurry was aged for 2 hours at atmospheric pressure and room temperature, before it was filtered. The solids isolated by filtration were washed with 200 mL of toluene and then dried under vacuum at 45° C. for 10 hours. A total of 44 g of the title compound was obtained in 80% yield (corrected for purity). ¹H NMR (400 MHz, DMSO-d₆) δ ppm 8.20 (br s, 3H), 3.37 (t, J=6.5 Hz, 2H), 3.14 (tt, J=10.4, 3.6 Hz, 1H), 2.93 (m, 1H), 2.00-1.90 (m, 4H), 1.50-1.12 (m, 10H), 0.86 (t, J=6.9 Hz, 3H).

### Example 4—Alternative Preparation of trans-4-(Pentyloxy)cyclohexanaminium Chloride (Compound of Formula (IV-A))

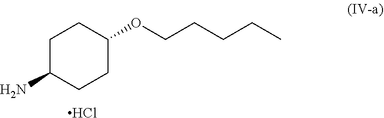

### Step 1—Preparation of trans-N,N-Dibenzyl-4-aminocyclohexanol (Compound of Formula (II))

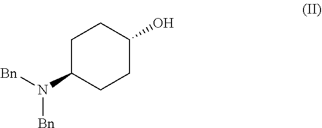 [SMILES: O[C@H]1CC[C@H](N(Cc2ccccc2)Cc2ccccc2)CC1]

To a slurry of 50 g of trans-4-aminocyclohexanol (Compound of Formula (I), 0.43 mol) and 90 g of potassium carbonate (0.65 mol; 1.5 equiv.) in 350 mL of DMF was slowly added 163.4 g of benzyl bromide (0.96 mol; 2.2 equiv.) while keeping the temperature in the range of 25−35° C. The mixture was stirred at this temperature for another 2 hours before 650 mL of water was added slowly over 9 hours while keeping the temperature at 25-35° C. The resulting slurry was stirred for 2 hours, allowed to cool to 20-25° C., filtered and the filtercake was washed with 150 mL of a 1:2 mixture of DMF and water. The solids were then slurried in 750 mL of water at 25° C. for 12 hours, filtered again, washed with 150 mL of water and 250 mL of heptane, and finally dried under vacuum at 45° C. for 12 hours. A total of 109.8 g of the compound of Formula (II) was obtained in 86% yield. ¹H NMR (400 MHz, CDCl₃) δ ppm 7.37-7.19 (m, 10H), 3.61 (s, 4H), 3.58-3.52 (m, 1H), 2.52-2.49 (m, 1H), 1.99 (d, J=11.5 Hz, 2H), 1.89 (d, J=12.5 Hz, 2H), 1.47-1.37 (m, 3H), 1.26-1.14 (m, 2H).

### Step 2—Preparation of trans-N,N-Dibenzyl-4-(pentyloxy)cyclohexanaminium chloride (Compound of Formula (III-a)

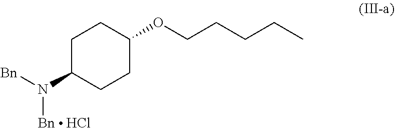 [SMILES: CCCCCO[C@H]1CC[C@H](N(Cc2ccccc2)Cc2ccccc2)CC1 ; Cl]

A solution of 100 g of trans-N,N-dibenzyl-4-aminocyclohexanol (Compound of Formula (II) from Step 1, 0.34 mol; 1.0 equiv.) and 210 g of 1-bromopentane (1.39 mol; 4.1 equiv.) in a mixture of 250 mL of DMSO and 250 mL of THF was slowly added a solution of 155.7 g of potassium tert-butoxide (1.38 mol; 4.1 equiv.) in 250 mL of DMSO and 250 mL of THF such that the temperature was maintained at 0-5° C. throughout. The resulting mixture was stirred at 0-5° C. for 1 hour and then 400 mL of water was added slowly. The resulting mixture was allowed to warm to at 25° C. and stirred for 30 min before the layers were separated. The organic layer was diluted with 800 mL of toluene and 200 mL of 12% NaCl solution was added. The mixture was agitated for 30 min and the layers were allowed to settle for 30 min. The organic layer was concentrated via distillation under reduced pressure to a total volume of 600-700 mL. Fresh toluene (400 mL) was added and the solution was concentrated again via distillation under reduced pressure to a total volume of 700-800 mL. To the resulting solution was slowly added 100 mL of a 4 M solution of HCl in EtOAc at 20° C. over 30 min. The resulting slurry was concentrated via distillation under reduced pressure to a total volume between 500-600 mL and then stirred at 20-30° C. for 2 hours. The slurry was filtered and the solids were washed with 200 mL of toluene. The solids were dried under vacuum at 45° C. for 16 hours. A total of 121.6 g of the compound of Formula (III-a) was obtained in 89% yield. ¹H NMR (400 MHz, DMSO-d₆) δ ppm 10.67 (s, 1H), 7.60 (m, 4H), 7.41 (m, 6H), 4.44 (dd, J=13.2, 4.5 Hz, 2H), 4.13 (dd, J=13.3, 5.9 Hz, 2H), 3.36 (t, J=6.3 Hz, 2H), 3.17 (m, 1H), 3.03 (m, 1H), 2.24 (d, J=11.1 Hz, 2H), 2.07 (d, J=10.2 Hz, 2H), 1.79-1.65 (m, 2H), 1.47-1.39 (m, 2H), 1.25 (m, 4H), 1.03-0.92 (m, 2H), 0.85 (t, J=6.6 Hz, 3H).

### Step 3—Preparation of trans-4-(Pentyloxy)cyclohexanaminium chloride (Compound of Formula (IV-a)

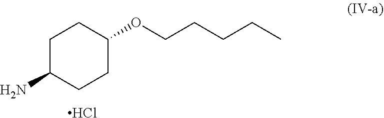

A mixture of 80.0 g of trans-N,N-dibenzyl-4-(pentyloxy)cyclohexanaminium chloride (Compound of Formula (III-a) from Step 2, 0.20 mol) and 4.8 g of 10 wt % Pd/C catalyst (45 wt % dry) in 400 mL of ethanol was hydrogenated at 50° C. under a hydrogen pressure of 0.8 MPa for 10 hours. The mixture was allowed to cool to 50° C. and then filtered over Celite. The cake was washed with 160 mL of ethanol. The combined filtrates were concentrated via distillation under reduced pressure to a total volume between 80-160 mL. A total of 680 mL of toluene was added in portions whilst the total volume of the mixture was maintained between 320-400 mL via distillation under reduced pressure. The temperature was adjusted to 10° C. and the slurry was stirred for 3 hours before it was filtered. The solids were washed with 160 mL of toluene at 10° C. and then dried under vacuum at 45° C. for 16 hours. A total of 37.5 g of the compound of Formula (IV-a) was obtained in 84% yield. ¹H NMR (400 MHz, DMSO-d₆) δ ppm 8.20 (br s, 3H), 3.37 (t, J=6.5 Hz, 2H), 3.14 (tt, J=10.4, 3.6 Hz, 1H), 2.93 (m, 1H), 2.00-1.90 (m, 4H), 1.50-1.12 (m, 10H), 0.86 (t, J=6.9 Hz, 3H).

### Example 5—Preparation of 5,7-Dimethyl-N-((1S*4S)-4-(pentyloxy)cyclohexyl)pyrazolo[1,5-a]pyrimidine-3-carboxamide (Compound of Formula (VIII)

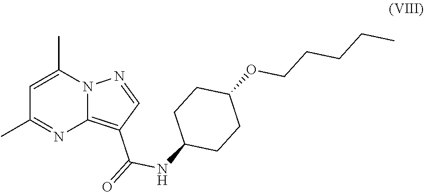 [SMILES: CCCCCO[C@H]1CC[C@H](NC(=O)c2cnn3c(C)cc(C)nc23)CC1]

### Step 1—Preparation of Ethyl 5,7-dimethylpyrazolo[1,5-a]pyrimidine-3-carboxylate (Compound of Formula (VI))

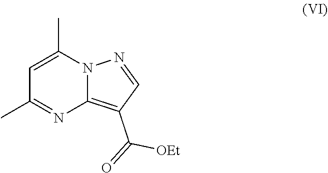 [SMILES: CCOC(=O)c1cnn2c(C)cc(C)nc12]

A mixture of 50.0 g of ethyl 3-amino-1H-pyrazole-4-carboxylate (0.32 mol; 1.0 equiv.), 13.1 g of glacial acetic acid (0.22 mol; 0.68 equiv.) and 36.1 g of pentane-2,4-dione (0.36 mol, 1.1 equiv.) in 300 mL of toluene was heated to 50° C. for 3 h. The resulting mixture was diluted with 100 mL of toluene and then concentrated via distillation under reduced pressure to a total volume of approximately 170 mL. The resulting mixture was heated to 65° C. until a clear solution was obtained before 575 mL of heptane was added over 1 hour. The temperature of the resulting mixture was allowed to gradually cool to 0-5° C. over 6 hours. The solids were filtered, washed with 100 mL of a mixture of heptane/toluene 9:1, 100 mL of heptane, and dried under vacuum at 40° C. overnight. A total of 64.6 g of the compound of Formula (VI) was obtained as an off-white solid (92% yield). ¹H NMR (400 MHz, DMSO-d₆) δ ppm 8.51 (s, 1H), 7.09 (d, J=0.9 Hz, 1H), 4.27 (q, J=7.1 Hz, 2H), 2.70 (d, J=0.9 Hz, 3H), 2.57 (s, 3H), 1.31 (t, J=7.1 Hz, 3H). ¹³C NMR (151 MHz, DMSO-d₆) δ ppm 162.7, 162.2, 147.4, 147.0, 146.9, 111.1, 101.3, 59.8, 25.0, 17.0, 14.9. LCMS: 220.17 (M+H)⁺.

### Step 2—Preparation of 5,7-Dimethylpyrazolo[1,5-a]pyrimidine-3-carboxylic Acid (Compound of Formula (VII))

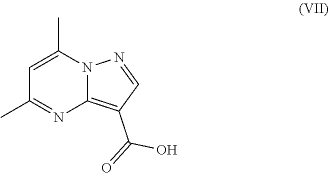

A mixture of 40.0 g of ethyl 5,7-dimethylpyrazolo[1,5-a]pyrimidine-3-carboxylate (Compound of Formula (VI) from Step 1, 0.18 mol) and 9.57 g of 3M NaOH solution (0.24 mol, 13.1 equiv.) in 320 mL of water was heated to 60° C. for 3 hours. The reaction mixture was allowed to cool to 45° C. and 26.8 g of conc. HCl (37%, 0.27 mol, 1.49 equiv.) was added dropwise until the pH reached a range of 1-2. The solution was slowly cooled to 0-5° C. over 4 hours and kept at this temperature for another hour. The solids were filtered, washed with water (2×40 mL), and dried under vacuum at 50° C. for 60 hours to give 33.6 g of the compound of Formula (VII) as a white solid (94% yield; corrected for wt % purity). ¹H NMR (600 MHz, DMSO-d₆) δ ppm 12.20 (br. s, 1H), 8.48 (s, 1H), 7.06 (s, 1H), 2.69 (s, 3H), 2.55 (s, 3H). ¹³C NMR (151 MHz, DMSO-d₆) δ ppm 163.8, 162.4, 147.5, 147.2, 147.0, 110.9, 102.0, 24.9, 17.0. LCMS: 192.12 (M+H)⁺.

### Step 3—Preparation of 5,7-Dimethyl-N-((1S*4S)-4-(pentyloxy)cyclohexyl)pyrazolo[1,5-a]pyrimidine-3-carboxamide (Compound of Formula (VIII)

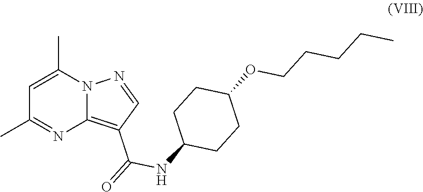 [SMILES: CCCCCO[C@H]1CC[C@H](NC(=O)c2cnn3c(C)cc(C)nc23)CC1]

A solution of 25.0 g of 5,7-dimethylpyrazolo[1,5-a]pyrimidine-3-carboxylic acid (Compound of Formula (VII) from Step 2, 98.4 wt % pure; 0.129 mol), 33.8 g of DIPEA (0.261 mol; 2.0 equiv.) 33.8 g of HOPO (0.144 mol; 1.1 equiv.) and 27.6 g of EDC (0.144 mol; 1.1 equiv.) in 125 mL of DMF was stirred at 25° C. for 2 hours before 28.6 g of trans-4-(pentyloxy)cyclohexanaminium chloride (Compound of Formula (IV-a) from Example 4, 0.129 mol, 1.0 equiv.) was added portion wise such that the temperature was kept constant. The resulting mixture was aged at 25° C. overnight before 125 mL of water was added dropwise over 4 hours whilst the temperature was maintained at approximately 25° C. The resulting slurry was stirred at 25° C. for another 2 hours and then filtered. The solids were washed with a 1:1 mixture of DMF/water (2×50 mL). The resulting wet cake was dissolved in 195 mL of DMF at 40° C. and the temperature of the resulting solution was adjusted to 25° C. before 125 mL of water was added dropwise over 4 hours. The resulting slurry was stirred at 25° C. for 3 hours and then filtered. The solids were washed with a 1:1 mixture of DMF and water (2×50 mL) and then dried overnight in vacuum at 55° C. to give 42.0 g of the title compound as an off-white solid (91% yield, corrected for purity of starting material and product). The crystal form was a mixture of Forms A and B by XRPD. ¹H NMR (600 MHz, DMSO-d₆) δ ppm 8.46 (s, 1H), 7.99 (d, J=7.6 Hz, 1H), 7.09 (s, 1H), 3.87-3.73 (m, 1H), 3.38 (t, J=6.6 Hz, 2H), 3.30-3.24 (m, 1H), 2.72 (s, 3H), 2.59 (s, 3H), 2.02-1.89 (m, 2H), 1.52-1.41 (m, 2H), 1.39-1.22 (m, 8H), 0.89-0.82 (m, 3H). ¹³C NMR (151 MHz, DMSO-d₆) δ ppm 161.3, 160.6, 147.0, 145.1, 144.8, 109.9, 104.1, 75.7, 67.2, 46.5, 29.9, 29.3, 28.0, 24.4, 22.0, 16.4, 13.9. LCMS: 359.38 (M+H)⁺.

### Example 6—Preparation of Crystalline Polymorphic Form B of 5,7-Dimethyl-N-((1S*,4S)-4-(pentyloxy)cyclohexyl)pyrazolo[1,5-a]pyrimidine-3-carboxamide (Compound of Formula (VIII))

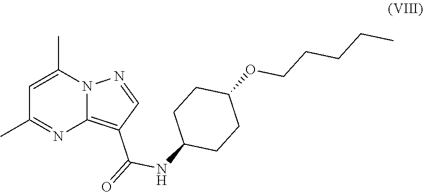 [SMILES: CCCCCO[C@H]1CC[C@H](NC(=O)c2cnn3c(C)cc(C)nc23)CC1]

### Step 1—Preparation of Ethyl 5,7-dimethylpyrazolo[1,5-a]pyrimidine-3-carboxylate (Compound of Formula (VI))

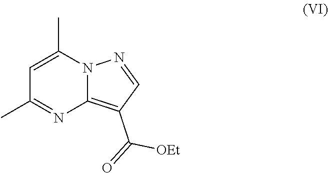 [SMILES: CCOC(=O)c1cnn2c(C)cc(C)nc12]

Ethyl 3-amino-1H-pyrazole-4-carboxylate (2.0 kg, 12.9 mol) was suspended in toluene (11.6 L) under nitrogen and acetic acid (500 ml, 8.7 mol, 0.68 equiv.) was added. Pentane-2,4-dione (1.48 L, 14.5 mol, 1.12 equiv.) was added dropwise over 10 min at ambient temperature. Toluene (400 ml) was used to rinse the addition funnel. The mixture was heated to an internal temperature of 48-49° C. for 3 h. Upon completion of the reaction the mixture was partially concentrated under reduced pressure and at an internal temperature of 35° C. to a total volume of approximately 4 L. Solids started to precipitate when the total volume reached approximately 6 L. The residue was heated to 60° C. while heptane (12 L) was added dropwise over 1 h to cause the precipitation of a white solid. The mixture was stirred at 60° C. for 30 min, then cooled down to 18-20° C. over 1 h. The resulting slurry was stirred overnight at ambient temperature. The solids were filtered, washed with a mixture of heptane/toluene 9:1 (4 L), heptane (4 L), and dried under vacuum at 40° C. overnight. A total of 2.641 kg of the compound of Formula (VI) was obtained as an off-white solid (93% yield, uncorrected for purity of the starting material and product). ¹H NMR (400 MHz, DMSO-d₆) δ ppm 8.51 (s, 1H), 7.09 (d, J=0.9 Hz, 1H), 4.27 (q, J=7.1 Hz, 2H), 2.70 (d, J=0.9 Hz, 3H), 2.57 (s, 3H), 1.31 (t, J=7.1 Hz, 3H). ¹³C NMR (151 MHz, DMSO-d₆) δ ppm 162.7, 162.2, 147.4, 147.0, 146.9, 111.1, 101.3, 59.8, 25.0, 17.0, 14.9. LCMS: 220.17 (M+H)⁺.

### Step 2—Preparation of 5,7-Dimethylpyrazolo[1,5-a]pyrimidine-3-carboxylic Acid (Compound of Formula (VII))

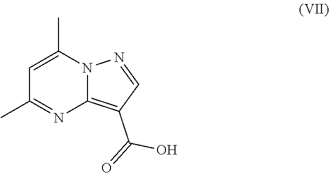

Ethyl 5,7-dimethylpyrazolo[1,5-a]pyrimidine-3-carboxylate (Compound of Formula (VI) from Step 1, 1.9 kg, 8.67 mol) was suspended in water (15.2 L) under nitrogen and 3M NaOH solution (3.8 L, 11.4 mol, 1.31 equiv.) was added. The suspension was heated to 60° C. and the resulting solution was stirred at 60° C. for 2.5 h. The reaction mixture was allowed to cool to 45° C. over 30 min, and then conc. HCl (37%, 1.08 L, 12.93 mol, 1.49 equiv.) was added dropwise over 10 min until the pH reached approximately 1.0. The solution was allowed to cool to 20° C. over 1 h. The resulting slurry was stirred at 20° C. for 30 min and then cooled to 0° C. over 30 min and aged at this temperature for 1 h. The solids were filtered, washed with water (2×1.9 L), and dried under vacuum at 50° C. for 90 h to give 1.64 kg of the compound of Formula (VII) as a white solid (94% yield, corrected for purity of the product). ¹H NMR (600 MHz, DMSO-d₆) δ ppm 12.20 (br. s, 1H), 8.48 (s, 1H), 7.06 (s, 1H), 2.69 (s, 3H), 2.55 (s, 3H). ¹³C NMR (151 MHz, DMSO-d₆) δ ppm 163.8, 162.4, 147.5, 147.2, 147.0, 110.9, 102.0, 24.9, 17.0. LCMS: 192.12 (M+H)⁺.

### Step 3—Preparation of Crystalline Polymorphic Form B of 5,7-Dimethyl-N-((1S*4S)-4-(pentyloxy)cyclohexyl)pyrazolo[1,5-a]pyrimidine-3-carboxamide (Compound of Formula (VIII))

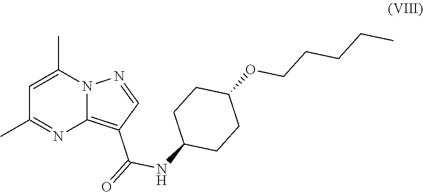 [SMILES: CCCCCO[C@H]1CC[C@H](NC(=O)c2cnn3c(C)cc(C)nc23)CC1]

5,7-Dimethylpyrazolo[1,5-a]pyrimidine-3-carboxylic acid (Compound of Formula (VII) from Step 2, 650 g, 96 wt % pure; 3.26 mol) was dissolved under nitrogen in DMF (5.2 L) at ambient temperature and HATU (1.27 kg, 3.34 mol, 1.0 equiv.) was added in one portion. The resulting mixture was cooled to 10° C. and DIPEA (2.12 L, 12.1 mol, 3.6 equiv.) was added dropwise over 50 min such that the temperature did not exceed 20° C. Trans-4-(pentyloxy)cyclohexanaminium chloride (Compound of Formula (IV-a) from Example 1, 765 g, 99 wt % pure; 3.41 mol, 1.05 equiv.) was added portion wise over 1 h such that the temperature was maintained below 20° C. The resulting mixture was aged for 1 h at 20° C. Water (10.4 L) was added dropwise over 75 min to the reaction mixture such that the internal temperature was maintained below 25° C. The resulting slurry was aged overnight at 20° C. and filtered. The solids were washed with a mixture of DMF/water 1:4 (5.2 L), followed by water (3×5.2 L), and then dried overnight in vacuum at 45° C. to give 1.12 kg of the compound of Formula (VIII) as an off-white solid (96% yield, corrected for purity of starting material and product). The crystal form was confirmed as Form B by XRPD and DSC. ¹H NMR (600 MHz, DMSO-d₆) δ ppm 8.46 (s, 1H), 7.99 (d, J=7.6 Hz, 1H), 7.09 (s, 1H), 3.87-3.73 (m, 1H), 3.38 (t, J=6.6 Hz, 2H), 3.30-3.24 (m, 1H), 2.72 (s, 3H), 2.59 (s, 3H), 2.02-1.89 (m, 2H), 1.52-1.41 (m, 2H), 1.39-1.22 (m, 8H), 0.89-0.82 (m, 3H). ¹³C NMR (151 MHz, DMSO-d₆) δ ppm 161.3, 160.6, 147.0, 145.1, 144.8, 109.9, 104.1, 75.7, 67.2, 46.5, 29.9, 29.3, 28.0, 24.4, 22.0, 16.4, 13.9. LCMS: 359.38 (M+H)⁺.

An X-ray powder diffractogram of the compound of Formula (VIII) is provided in FIG. 1. A differential scanning calorimetry curve of the compound of Formula (VIII) is provided in FIG. 2. The differential scanning calorimetry curve displayed endothermic events at about 90° C. and about 110° C. Tabulated characteristics of the X-ray powder diffractogram in FIG. 1 are provided below in Table 9, which lists diffraction angle 2θ, inter-planar distances d, and relative intensity (expressed as a percentage with respect to the most intense peak).

TABLE 9
X-RAY POWDER DIFFRACTOGRAM DATA OF CRYSTALLINE
POLYMORPHIC FORM B.

| Angle [2θ] | d-spacing [Å] | Relative Intensity [%] |
|---|---|---|
| 4.0 | 21.9 | 100.0 |
| 10.9 | 8.2 | 89.0 |
| 11.4 | 7.8 | 22.4 |
| 12.1 | 7.3 | 18.4 |
| 12.3 | 7.2 | 73.2 |
| 13.4 | 6.6 | 17.7 |
| 16.0 | 5.5 | 13.9 |
| 16.2 | 5.5 | 32.5 |
| 16.5 | 5.4 | 11.6 |
| 17.8 | 5.0 | 11.7 |
| 18.6 | 4.8 | 19.7 |
| 19.5 | 4.6 | 13.2 |
| 20.2 | 4.4 | 88.0 |
| 20.4 | 4.3 | 11.1 |
| 21.1 | 4.2 | 48.3 |
| 21.5 | 4.1 | 58.8 |
| 21.6 | 4.1 | 27.1 |
| 22.3 | 4.0 | 9.8 |
| 22.5 | 4.0 | 21.2 |
| 22.9 | 3.9 | 21.2 |
| 23.4 | 3.8 | 36.5 |
| 23.5 | 3.8 | 33.3 |
| 24.7 | 3.6 | 35.1 |
| 25.7 | 3.5 | 11.6 |
| 25.8 | 3.5 | 10.1 |
| 27.6 | 3.2 | 27.9 |
| 30.8 | 2.9 | 8.5 |
| 30.9 | 2.9 | 12.4 |

### Example 7—Alternative Preparation and Characterization of Crystalline Polymorphic Form B of 5,7-Dimethyl-N-((1S*,4S)-4-(pentyloxy)cyclohexyl)pyrazolo[1,5-A]pyrimidine-3-carboxamide (Compound of Formula (VIII))

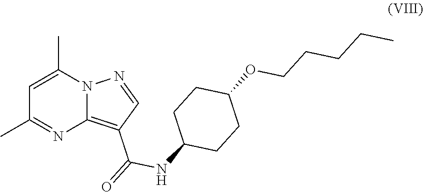 [SMILES: CCCCCO[C@H]1CC[C@H](NC(=O)c2cnn3c(C)cc(C)nc23)CC1]

A mixture of 42.0 g of 5,7-dimethyl-N-((1S*,4S)-4-(pentyloxy)cyclohexyl)pyrazolo[1,5-a]pyrimidine-3-carboxamide (Compound of Formula (VIII from Example 5) in 460 mL of MEK was stirred at 25° C. until the solids had completely dissolved. The resulting solution was filtered through a 10 μm line filter and then 230 mL of heptane was added slowly to the filtrate over 4 hours. The resulting solution was concentrated via distillation under reduced pressure at a temperature below 30° C. until the total volume had reached approximately 450 mL. Heptane portions of 150 mL each were added three more times and each time the mixture was concentrated via distillation under reduced pressure at a temperature below 30° C. until the total volume had reached approximately 450 mL again. The final slurry was stirred at 20° C. for 2 hours, then slowly cooled to 0-5° C. over 4 hours and stirred at this temperature for 6 hours. The cold slurry was filtered and the solids were washed with 75 mL of a cold 1:10 mixture of MEK and heptane, followed by 75 mL of cold heptane. The resulting off-white product was dried in vacuum at 30° C. for 16 hours to yield 37.8 g of the pure title compound (90% yield). The crystal form of these solids was confirmed as Form B by DSC and XRPD.

An X-ray powder diffractogram of the compound of Formula (VIII) is provided in FIG. 3. A differential scanning calorimetry curve of the compound of Formula (VIII) is provided in FIG. 4. The differential scanning calorimetry curve displayed endothermic events with onset values of about 89° C. and about 109° C. Tabulated characteristics of the X-ray powder diffractogram in FIG. 3 are provided below in Table 10, which lists diffraction angle 2θ and relative intensity (expressed as a percentage with respect to the most intense peak).

TABLE 10
X-RAY POWDER DIFFRACTOGRAM DATA OF
CRYSTALLINE POLYMORPHIC FORM B.

| Angle (2θ °) | Intensity % |
|---|---|
| 4.2 | 51.0 |
| 10.9 | 100 |
| 11.5 | 31.2 |
| 12.4 | 96.2 |
| 13.5 | 6.5 |
| 14.8 | 5.5 |
| 16.3 | 51.0 |
| 17.7 | 4.1 |
| 18.6 | 2.1 |
| 19.5 | 2.2 |
| 20.2 | 9.9 |
| 20.4 | 7.9 |
| 21.1 | 10.5 |
| 21.5 | 23.7 |
| 21.8 | 5.5 |
| 22.3 | 27.1 |
| 22.4 | 40.4 |
| 22.9 | 30.2 |
| 23.0 | 28.6 |
| 23.5 | 7.8 |
| 23.8 | 4.4 |
| 24.7 | 4.9 |
| 25.8 | 19.1 |
| 27.6 | 6.6 |
| 28.3 | 2.7 |
| 29.8 | 2.4 |

In addition, single crystals of crystalline polymorphic Form B of 5,7-dimethyl-N-((1S*, 4S)-4-(pentyloxy)cyclohexyl)pyrazolo[1,5-a]pyrimidine-3-carboxamide were analyzed by single crystal X-ray diffraction analysis. The unit cell parameters of crystalline polymorphic Form B and the data collection and structure refinement methods are shown in Table 11 and Table 12, respectively.

TABLE 11
UNIT CELL PARAMETERS OF CRYSTALLINE
POLYMORPHIC FORM B.

| Empirical formula | C₂₀H₃₀N₄O₂ | |
|---|---|---|
| Formula weight | 358.48 | |
| Temperature | 100.1(3) K | |
| Wavelength | 1.54178 Å | |
| Crystal size | 0.250 × 0.200 × 0.090 mm | |
| Crystal habit | colorless prism | |
| Crystal system | Monoclinic | |
| Space group | P2₁/c | |
| Unit cell dimensions | a = 5.49080(10) Å | α = 90° |
| | b = 43.1070(8) Å | β = 94.827(2)° |
| | c = 8.2570(2) Å | γ = 90° |

| | |
|---|---|
| Volume | 1947.43(7) Å³ |
| Z | 4 |
| Density (calculated) | 1.223 Mg/m³ |
| Absorption coefficient | 0.640 mm⁻¹ |
| F(000) | 776 |

TABLE 12
DATA COLLECTION AND STRUCTURE REFINEMENT METHODS
FOR CRYSTALLINE POLYMORPHIC FORM B.

| | |
|---|---|
| Diffractometer | SuperNova, Dual, Cu at zero, Atlas |
| Radiation source | SuperNova (Cu) X-ray Source, CuKα |
| Data collection method | omega scans |
| Theta range for data collection | 4.102 to 70.364° |
| Index ranges | −6 ≤ h ≤ 6, −50 ≤ k ≤ 52, −10 ≤ l ≤ 10 |
| Reflections collected | 36759 |
| Independent reflections | 3715 [R(int) = 0.0365] |
| Coverage of independent reflections | 100.00% |
| Absorption correction | Semi-empirical from equivalents |
| Max. and min. transmission | 1.00000 and 0.90289 |
| Structure solution technique | Direct Methods |
| Structure solution program | SHELXTL (Sheldrick, 2013) |
| Refinement technique | Full-matrix least-squares on F² |
| Refinement program | SHELXL-2013 (Sheldrick, 2013) |
| Function minimized | Σw(Fₒ² − F_{c}²)² |
| Data/restraints/parameters | 3715/0/242 |
| Goodness-of-fit on F2 | 1.046 |
| D/smax | 0.001 |
| Final R indices | |
| 3352 data; I > 2 s(I) | R1 = 0.0363, wR2 = 0.0856 |
| all data | R1 = 0.0411, wR2 = 0.0890 |
| Weighting scheme | w = 1/[σ² (Fₒ²) + (0.0382P)² + 0.8518P] |
| | where P = (Fₒ² + 2F_{c}²)/3 |
| Extinction coefficient | N/A |
| Largest diff. peak and hole | 0.205 and −0.212 eÅ⁻³ |

Atomic coordinates (×10⁴) and equivalent isotropic displacement parameters (Å²×10³) are shown in Table 13, below. U(eq) is defined as one third of the trace of the orthogonalized U^{ij} tensor.

TABLE 13
ATOMIC COORDINATES AND EQUIVALENT ISOTROPIC
ATOMIC DISPLACEMENT PARAMETERS FOR CRYSTALLINE
POLYMORPHIC FORM B.

| Atom | x/a | y/b | z/c | U(eq) |
|---|---|---|---|---|
| O1 | 1.12314(15) | 0.55025(2) | 0.62039(10) | 0.0210(2) |
| O2 | 0.48544(16) | 0.67803(2) | 0.78677(10) | 0.0227(2) |
| N1 | 0.46451(19) | 0.65085(2) | 0.54915(13) | 0.0186(2) |
| N2 | −0.14398(19) | 0.72535(2) | 0.55851(12) | 0.0209(2) |
| N3 | −0.12932(18) | 0.70996(2) | 0.41463(12) | 0.0174(2) |
| N4 | 0.11026(18) | 0.67146(2) | 0.29206(12) | 0.0174(2) |
| C1 | 1.6071(3) | 0.42980(3) | 0.92034(16) | 0.0276(3) |
| C2 | 1.5900(2) | 0.45876(3) | 0.81433(16) | 0.0239(3) |
| C3 | 1.3492(2) | 0.47620(3) | 0.81974(15) | 0.0207(3) |
| C4 | 1.3407(2) | 0.50558(3) | 0.71692(15) | 0.0214(3) |
| C5 | 1.0997(2) | 0.52264(3) | 0.71211(15) | 0.0211(3) |
| C6 | 0.9049(2) | 0.56846(3) | 0.59788(14) | 0.0183(2) |
| C7 | 0.9463(2) | 0.59175(3) | 0.46449(14) | 0.0199(3) |
| C8 | 0.8466(2) | 0.58531(3) | 0.75271(14) | 0.0193(3) |
| C9 | 0.7279(2) | 0.61354(3) | 0.43363(14) | 0.0191(2) |
| C10 | 0.6249(2) | 0.60669(3) | 0.72051(14) | 0.0187(2) |
| C11 | 0.6727(2) | 0.63040(3) | 0.58887(14) | 0.0176(2) |
| C12 | 0.3886(2) | 0.67310(3) | 0.64888(14) | 0.0173(2) |
| C13 | 0.1759(2) | 0.69091(3) | 0.57903(14) | 0.0174(2) |
| C14 | 0.0399(2) | 0.71345(3) | 0.65463(15) | 0.0201(3) |
| C15 | 0.0616(2) | 0.68890(3) | 0.42151(14) | 0.0164(2) |
| C16 | −0.2857(2) | 0.71368(3) | 0.27823(15) | 0.0194(3) |
| C17 | −0.4892(2) | 0.73634(3) | 0.28453(16) | 0.0231(3) |
| C18 | −0.2380(2) | 0.69580(3) | 0.14779(15) | 0.0204(3) |
| C19 | −0.0365(2) | 0.67507(3) | 0.15740(14) | 0.0191(2) |
| C20 | 0.0171(3) | 0.65578(3) | 0.01372(15) | 0.0262(3) |

Bond lengths (Å) are shown in Table 14, below.

TABLE 14
SELECTED BOND LENGTHS (Å)
FOR CRYSTALLINE POLYMORPHIC FORM B.

| Bond | Bond length (Å) | Bond | Bond length (Å) |
|---|---|---|---|
| O1—C5 | 1.4221(14) | O1—C6 | 1.4315(14) |
| O2—C12 | 1.2339(15) | N1—C12 | 1.3524(16) |
| N1—C11 | 1.4592(15) | N1—H1 | 0.868(17) |
| N2—C14 | 1.3333(17) | N2—N3 | 1.3689(14) |
| N3—C16 | 1.3671(16) | N3—C15 | 1.3843(15) |
| N4—C19 | 1.3262(16) | N4—C15 | 1.3518(15) |
| C1—C2 | 1.5233(18) | C2—C3 | 1.5247(17) |
| C3—C4 | 1.5232(17) | C4—C5 | 1.5115(17) |
| C6—C7 | 1.5215(16) | C6—C8 | 1.5276(16) |
| C7—C9 | 1.5275(17) | C8—C10 | 1.5321(16) |
| C9—C11 | 1.5259(16) | C10—C11 | 1.5307(16) |
| C12—C13 | 1.4740(17) | C13—C15 | 1.3985(17) |
| C13—C14 | 1.4034(17) | C16—C18 | 1.3677(18) |
| C16—C17 | 1.4879(17) | C18—C19 | 1.4192(17) |
| C19—C20 | 1.4980(17) | | |

Bond angles (°) are shown in Table 15, below.

TABLE 15
SELECTED BOND ANGLES (°) FOR
CRYSTALLINE POLYMORPHIC FORM B.

| | Bond angle (°) | | Bond angle (°) |
|---|---|---|---|
| C5—O1—C6 | 114.57(9) | C12—N1—C11 | 124.57(10) |
| C12—N1—H1 | 117.6(10) | C11—N1—H1 | 117.6(10) |
| C14—N2—N3 | 103.49(10) | C16—N3—N2 | 125.41(10) |
| C16—N3—C15 | 122.13(10) | N2—N3—C15 | 112.44(9) |
| C19—N4—C15 | 116.82(10) | C1—C2—C3 | 113.52(11) |
| C4—C3—C2 | 112.30(10) | C5—C4—C3 | 113.90(10) |
| O1—C5—C4 | 107.65(10) | O1—C6—C7 | 106.36(9) |
| O1—C6—C8 | 112.60(9) | C7—C6—C8 | 110.29(10) |
| C6—C7—C9 | 111.32(10) | C6—C8—C10 | 111.11(10) |
| C11—C9—C7 | 111.17(10) | C11—C10—C8 | 110.09(10) |
| N1—C11—C9 | 107.83(9) | N1—C11—C10 | 112.64(10) |
| C9—C11—C10 | 109.67(9) | O2—C12—N1 | 123.57(11) |
| O2—C12—C13 | 122.48(11) | N1—C12—C13 | 113.94(10) |
| C15—C13—C14 | 104.04(10) | C15—C13—C12 | 127.62(11) |
| C14—C13—C12 | 128.34(11) | N2—C14—C13 | 113.97(11) |
| N4—C15—N3 | 122.12(10) | N4—C15—C13 | 131.82(11) |
| N3—C15—C13 | 106.06(10) | N3—C16—C18 | 115.71(11) |
| N3—C16—C17 | 118.01(11) | C18—C16—C17 | 126.29(11) |
| C16—C18—C19 | 120.74(11) | N4—C19—C18 | 122.44(11) |
| N4—C19—C20 | 116.93(11) | C18—C19—C20 | 120.62(11) |

Torsion angles (°) are shown in Table 16, below.

TABLE 16
SELECTED TORSION ANGLES (°)
FOR CRYSTALLINE POLYMORPHIC FORM B.

| | Torsion angle (°) | | Torsion angle (°) |
|---|---|---|---|
| C14—N2—N3—C16 | 178.11(11) | C14—N2—N3—C15 | −0.21(12) |
| C1—C2—C3—C4 | 178.16(11) | C2—C3—C4—C5 | 176.99(11) |
| C6—O1—C5—C4 | 177.20(9) | C3—C4—C5—O1 | 176.69(10) |
| C5—O1—C6—C7 | −165.88(10) | C5—O1—C6—C8 | 73.22(12) |
| O1—C6—C7—C9 | −177.81(9) | C8—C6—C7—C9 | −55.44(13) |
| O1—C6—C8—C10 | 175.15(9) | C7—C6—C8—C10 | 56.52(13) |
| C6—C7—C9—C11 | 56.66(13) | C6—C8—C10—C11 | −58.23(13) |
| C12—N1—C11—C9 | −168.67(11) | C12—N1—C11—C10 | 70.18(14) |
| C7—C9—C11—N1 | 179.47(9) | C7—C9—C11—C10 | −57.54(12) |
| C8—C10—C11—N1 | 178.20(9) | C8—C10—C11—C9 | 58.10(12) |
| C11—N1—C12—O2 | −1.58(18) | Cl 1—N1—C12—C13 | 179.13(10) |
| O2—C12—C13—C15 | 175.29(11) | N1—C12—C13—C15 | −5.42(17) |
| O2—C12—C13—C14 | −5.47(19) | N1—C12—C13—C14 | 173.82(11) |
| N3—N2—C14—C13 | 0.34(13) | C15—C13—C14—N2 | −0.34(14) |
| C12—C13—C14—N2 | −179.72(11) | C19—N4—C15—N3 | −1.00(16) |
| C19—N4—C15—C13 | 179.85(12) | C16—N3—C15—N4 | 2.28(17) |
| N2—N3—C15—N4 | −179.33(10) | C16—N3—C15—C13 | −178.37(10) |
| N2—N3—C15—C13 | 0.01(13) | C14—C13—C15—N4 | 179.44(12) |
| C12—C13—C15—N4 | −1.2(2) | C14—C13—C15—N3 | 0.18(12) |
| C12—C13—C15—N3 | 179.56(11) | N2—N3—C16—C18 | −179.68(11) |
| C15—N3—C16—C18 | −1.51(16) | N2—N3—C16—C17 | 0.26(17) |
| C15—N3—C16—C17 | 178.42(10) | N3—C16—C18—C19 | −0.32(17) |
| C17—C16—C18—C19 | 179.76(11) | C15—N4—C19—C18 | −0.86(17) |
| C15—N4—C19—C20 | 179.96(10) | C16—C18—C19—N4 | 1.57(18) |
| C16—C18—C19—C20 | −179.29(11) | | |

Anisotropic displacement parameters (Å²) are shown in Table 17, below. The anisotropic displacement factor exponent may be expressed in the form: −2π²[h²a*²U¹¹+ . . . +2 h k a*b*U¹²].

TABLE 17
ANISOTROPIC DISPLACEMENT PARAMETERS (Å²) FOR CRYSTALLINE
POLYMORPHIC FORM B.

| Atom | U11 | U22 | U33 | U23 | U13 | U12 |
|---|---|---|---|---|---|---|
| O1 | 0.0194(4) | 0.0188(4) | 0.0252(4) | 0.0040(3) | 0.0044(3) | 0.0020(3) |
| O2 | 0.0242(5) | 0.0258(5) | 0.0171(4) | −0.0017(3) | −0.0037(3) | −0.0008(4) |
| N1 | 0.0216(5) | 0.0183(5) | 0.0151(5) | −0.0001(4) | −0.0037(4) | 0.0012(4) |
| N2 | 0.0243(5) | 0.0194(5) | 0.0193(5) | −0.0026(4) | 0.0031(4) | −0.0005(4) |
| N3 | 0.0191(5) | 0.0157(5) | 0.0174(5) | 0.0009(4) | 0.0017(4) | −0.0009(4) |
| N4 | 0.0201(5) | 0.0158(5) | 0.0160(5) | 0.0003(4) | 0.0004(4) | −0.0015(4) |
| Cl | 0.0366(7) | 0.0238(7) | 0.0226(6) | 0.0017(5) | 0.0032(5) | 0.0073(6) |
| C2 | 0.0277(7) | 0.0228(6) | 0.0218(6) | 0.0017(5) | 0.0050(5) | 0.0050(5) |
| C3 | 0.0228(6) | 0.0195(6) | 0.0201(6) | −0.0001(5) | 0.0031(5) | 0.0003(5) |
| C4 | 0.0227(6) | 0.0204(6) | 0.0215(6) | 0.0008(5) | 0.0042(5) | 0.0016(5) |
| C5 | 0.0217(6) | 0.0185(6) | 0.0233(6) | 0.0025(5) | 0.0036(5) | 0.0002(5) |
| C6 | 0.0158(6) | 0.0184(6) | 0.0205(6) | 0.0011(5) | 0.0006(4) | 0.0005(4) |
| C7 | 0.0210(6) | 0.0204(6) | 0.0186(6) | 0.0002(5) | 0.0035(5) | −0.0010(5) |
| C8 | 0.0203(6) | 0.0204(6) | 0.0169(6) | 0.0031(5) | 0.0004(4) | 0.0019(5) |
| C9 | 0.0209(6) | 0.0198(6) | 0.0165(6) | 0.0021(4) | 0.0007(4) | −0.0017(5) |
| C10 | 0.0188(6) | 0.0202(6) | 0.0171(6) | 0.0009(5) | 0.0016(4) | 0.0004(5) |
| C11 | 0.0178(6) | 0.0168(6) | 0.0178(6) | 0.0009(4) | −0.0015(4) | −0.0009(4) |
| C12 | 0.0181(6) | 0.0169(6) | 0.0169(6) | 0.0020(4) | 0.0010(4) | −0.0041(4) |
| C13 | 0.0196(6) | 0.0160(6) | 0.0165(6) | 0.0010(4) | 0.0006(4) | −0.0034(4) |
| C14 | 0.0239(6) | 0.0193(6) | 0.0171(6) | −0.0020(5) | 0.0013(5) | −0.0029(5) |
| C15 | 0.0169(6) | 0.0141(5) | 0.0183(6) | 0.0023(4) | 0.0022(4) | −0.0021(4) |
| C16 | 0.0187(6) | 0.0181(6) | 0.0213(6) | 0.0063(5) | 0.0011(5) | −0.0027(5) |
| C17 | 0.0209(6) | 0.0227(6) | 0.0258(6) | 0.0052(5) | 0.0018(5) | 0.0019(5) |
| C18 | 0.0217(6) | 0.0206(6) | 0.0182(6) | 0.0039(5) | −0.0021(5) | −0.0014(5) |
| C19 | 0.0219(6) | 0.0180(6) | 0.0172(6) | 0.0022(5) | 0.0001(5) | −0.0037(5) |
| C20 | 0.0315(7) | 0.0281(7) | 0.0180(6) | −0.0028(5) | −0.0027(5) | 0.0044(5) |

Hydrogen atom coordinates and isotropic atomic displacement parameters (Å²) are shown in Table 18, below.

TABLE 18
HYDROGEN ATOM COORDINATES AND ISOTROPIC
DISPLACEMENT PARAMETERS (Å²) FOR
CRYSTALLINE POLYMORPHIC FORM B.

| Atom | x/a | y/b | z/c | U |
|---|---|---|---|---|
| H1A | 1.6033 | 0.4358 | 1.0346 | 0.041 |
| H1B | 1.7604 | 0.4189 | 0.9056 | 0.041 |
| H1C | 1.4688 | 0.4161 | 0.889 | 0.041 |
| H2A | 1.61 | 0.4527 | 0.7005 | 0.029 |
| H2B | 1.7262 | 0.4729 | 0.8502 | 0.029 |
| H3A | 1.2128 | 0.4624 | 0.7801 | 0.025 |
| H3B | 1.3261 | 0.4818 | 0.9338 | 0.025 |
| H4A | 1.4725 | 0.5197 | 0.7604 | 0.026 |
| H4B | 1.3736 | 0.5 | 0.6045 | 0.026 |
| H5A | 0.9669 | 0.5095 | 0.6606 | 0.025 |
| H5B | 1.0601 | 0.5278 | 0.8238 | 0.025 |
| H6 | 0.7647 | 0.5547 | 0.5608 | 0.022 |
| H7A | 0.9734 | 0.5805 | 0.363 | 0.024 |
| H7B | 1.0948 | 0.6041 | 0.4964 | 0.024 |
| H8A | 0.99 | 0.5977 | 0.7945 | 0.023 |
| H8B | 0.8127 | 0.5699 | 0.8368 | 0.023 |
| H9A | 0.7632 | 0.6289 | 0.3498 | 0.023 |
| H9B | 0.5827 | 0.6014 | 0.3919 | 0.023 |
| H10A | 0.4788 | 0.5942 | 0.685 | 0.022 |
| H10B | 0.5926 | 0.6176 | 0.8219 | 0.022 |
| H11 | 0.8176 | 0.6433 | 0.6275 | 0.021 |
| H14 | 0.0756 | 0.7197 | 0.7644 | 0.024 |
| H17A | −0.5761 | 0.7324 | 0.3814 | 0.035 |
| H17B | −0.6026 | 0.7341 | 0.1871 | 0.035 |
| H17C | −0.4228 | 0.7575 | 0.2892 | 0.035 |
| H18 | −0.3408 | 0.6973 | 0.0496 | 0.024 |
| H20A | 0.1761 | 0.6618 | −0.0223 | 0.039 |
| H20B | −0.1104 | 0.6591 | −0.0749 | 0.039 |
| H20C | 0.0207 | 0.6338 | 0.0443 | 0.039 |
| H1 | 0.391(3) | 0.6496(3) | 0.452(2) | 0.025(4) |

Site occupancy factors (sof) that deviate from unity shown in Table 19, below.

TABLE 19
SITE OCCUPANCY FACTORS THAT DEVIATE FROM
UNITY FOR CRYSTALLINE POLYMORPHIC FORM B.

| Atom | sof | Atom | sof | Atom | sof |
|---|---|---|---|---|---|
| O1 | 1 | O2 | 1 | N1 | 1 |
| N2 | 1 | N3 | 1 | N4 | 1 |
| C1 | 1 | H1A | 1 | H1B | 1 |
| H1C | 1 | C2 | 1 | H2A | 1 |
| H2B | 1 | C3 | 1 | H3A | 1 |
| H3B | 1 | C4 | 1 | H4A | 1 |
| H4B | 1 | C5 | 1 | H5A | 1 |
| H5B | 1 | C6 | 1 | H6 | 1 |
| C7 | 1 | H7A | 1 | H7B | 1 |
| C8 | 1 | H8A | 1 | H8B | 1 |
| C9 | 1 | H9A | 1 | H9B | 1 |
| C10 | 1 | H10A | 1 | H10B | 1 |
| C11 | 1 | H11 | 1 | C12 | 1 |
| C13 | 1 | C14 | 1 | H14 | 1 |
| C15 | 1 | C16 | 1 | C17 | 1 |
| H17A | 1 | H17B | 1 | H17C | 1 |
| C18 | 1 | H18 | 1 | C19 | 1 |
| C20 | 1 | H20A | 1 | H20B | 1 |
| H20C | 1 | Hl | 1 | | |

Selected hydrogen bond information (Å and °) shown in Table 20, below.

TABLE 20
SELECTED HYDROGEN BOND FORMATION (Å and
°) FOR CRYSTALLINE POLYMORPHIC FORM B.

| D-H . . . A | d(D-H) | d(H . . . A) | d(D . . . A) | <(DHA) |
|---|---|---|---|---|
| N1—H1 . . . N4 | 0.868(17) | 2.161(16) | 2.8955(14) | 142.1(14) |

### Example 8—Preparation of Crystalline Polymorphic Form of 5,7-Dimethyl-N-((1S*4S)-4-(pentyloxy)cyclohexyl)pyrazolo[1,5-a]pyrimidine-3-carboxamide (Compound of Formula (VIII))

5,7-Dimethyl-N-((1S*,4S)-4-(pentyloxy)cyclohexyl)pyrazolo[1,5-a]pyrimidine-3-carboxamide (Compound of Formula (VI) from Example 6, 71 g, 0.20 mol) in ethyl acetate (177.5 mL) was stirred at room temperature for 15 min and then hexane (177.5 mL) was added slowly over 10 min. The temperature of the mixture was slowly increased to 60° C. and kept at this temperature for one hour resulting in a hazy solution. The resulting mixture was then allowed to cool to room temperature, and then stirred for another one hour before hexane (710 mL) was added dropwise over five hours under gentle stirring. The resulting slurry was stirred overnight at room temperature and filtered. The product was dried in vacuo in an oven at a temperature of 50° C. to afford the title compound as a white solid (52.55 g, 74%). The crystal form was confirmed as Form A by XRPD and DSC. ¹H NMR (500 MHz, DMSO-d₆) δ 8.47 (s, 1H), 8.00 (d, J=8.0 Hz, 1H), 7.10 (s, 1H), 3.82-3.80 (m, 1H), 3.39 (t, J=6.0 Hz, 2H), 3.30-3.27 (m, 1H), 2.72 (s, 3H), 2.60 (s, 3H), 1.98-1.95 (m, 4H), 1.50-1.45 (m, 2H), 1.39-1.27 (m, 8H), 0.87 (t, J=7.0 Hz, 3H). LC-MS m/z: 359.2 [M+H]⁺. HPLC: Purity (214 nm): >99%; t_{R}=9.52 min.

### Example 9—Alternative Preparation and Characterization of Crystalline Polymorphic Form A of 5,7-Dimethyl-N-((1S*,4S)-4-(pentyloxy)cyclohexyl)pyrazolo[1,5-a]pyrimidine-3-carboxamide (Compound of Formula (VII))

Crystalline polymorphic Form B of 5,7-dimethyl-N-((1S*,4S)-4-(pentyloxy)cyclohexyl)pyrazolo[1,5-a]pyrimidine-3-carboxamide (Compound of Formula (VII) from Example 6, 750 g, 2.09 mol) was dissolved in EtOAc (7.5 L) at 40° C. The solution was passed through a CUNO filter, which was subsequently washed with additional EtOAc (2×7.5 L; 1×4 L). The colorless eluents were combined and passed through a 5 μm polishing filter. The line was washed with EtOAc (2.0 L). The combined filtrates were concentrated under reduced pressure to a total volume of approximately 1.7 L and then heptane (1.85 L) was added. The resulting mixture was heated to 75° C. until the suspension had become a clear, pale yellow solution. This solution was cooled to 50° C. over 30 min and a seed of crystalline polymorphic Form A of 5,7-dimethyl-N-(1S*, 4S)-4-(pentyloxy)cyclohexyl)pyrazolo[1,5-a]pyrimidine-3-carboxamide (1.5 g; 0.2 wt %) was added. The resulting mixture was stirred at 45-48° C. for 1 hr and then cooled to 20° C. over 20 min and stirred at this temperature for another 1 hr. Additional heptane (4.5 L) was added dropwise over 1 hr and the resulting suspension was aged overnight at 20° C. The slurry was filtered. The solids were washed with EtOAc/heptane 1:9 (1.5 L), heptane (0.75 L), and then dried overnight at 40° C. under vacuum to give 666 g of the title compound as a white solid (89% yield). The crystal form was confirmed as Form A by XRPD and DSC. ¹H NMR (600 MHz, DMSO-d₆) δ ppm 8.46 (s, 1H), 7.99 (d, J=7.7 Hz, 1H), 7.08 (s, 1H), 3.81 (m, 1H), 3.38 (t, J=6.5 Hz, 2H), 3.27 (m, 1H), 2.71 (s, 3H), 2.59 (s, 3H), 1.96 (m, 4H), 1.47 (quin, J=6.9 Hz, 2H), 1.31 (m, 8H), 0.86 (m, 3H). 13C NMR (151 MHz, DMSO-d₆) δ ppm 161.3, 160.6, 147.0, 145.1, 144.8, 109.9, 104.1, 75.7, 67.2, 46.5, 29.9, 29.3, 28.0, 24.4, 22.0, 16.4, 13.9. LCMS: 359.38 (M+H)⁺.

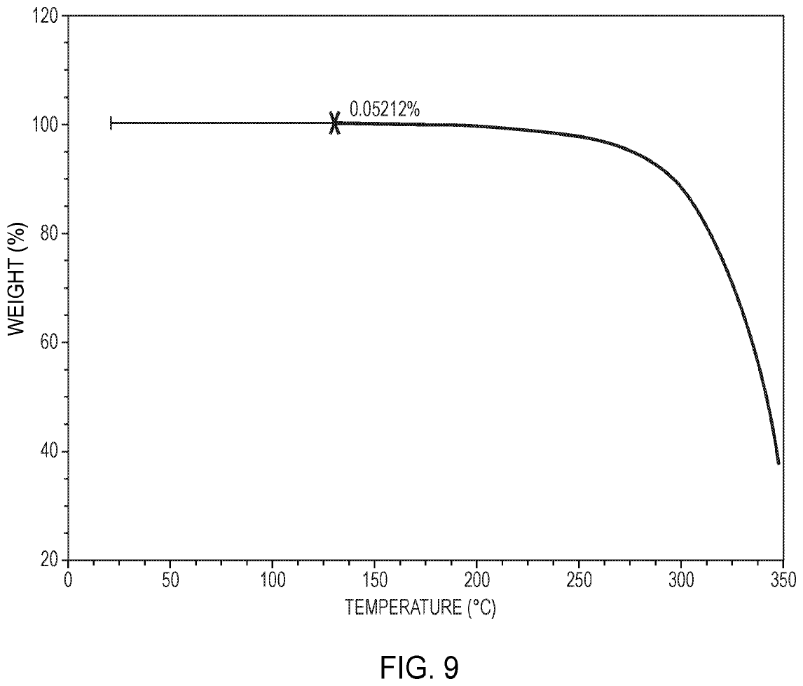

An X-ray powder diffractogram of the title compound is provided in FIG. 5. A differential scanning calorimetry curve of the title compound is provided in FIG. 6. The differential scanning calorimetry curve displayed an endothermic event at about 113° C. A thermogravimetric analysis curve of the title compound is provided in FIG. 9. The thermogravimetric analysis curve displayed a weight loss of approximately 0.2 wt % in the region of 20° C. to 180° C., confirming low levels of residual water in the title compound. Tabulated characteristics of the X-ray powder diffractogram in FIG. 5 are provided below in Table 21, which lists diffraction angle 2θ, inter-planar distances d, and relative intensity (expressed as a percentage with respect to the most intense peak).

TABLE 21
X-RAY POWDER DIFFRACTOGRAM DATA OF
CRYSTALLINE POLYMORPHIC FORM A.

| Angle [2θ] | d-spacing [Å] | Relative Intensity [%] |
|---|---|---|
| 5.7 | 15.5 | 61.3 |
| 10.9 | 8.1 | 11.2 |
| 11.5 | 7.7 | 29.8 |
| 11.8 | 7.5 | 40.0 |
| 12.8 | 6.9 | 32.8 |
| 13.6 | 6.5 | 26.9 |
| 14.4 | 6.1 | 25.7 |
| 14.7 | 6.0 | 29.0 |
| 15.8 | 5.6 | 18.4 |
| 17.2 | 5.2 | 80.3 |
| 17.9 | 5.0 | 9.9 |
| 18.7 | 4.7 | 100.0 |
| 19.6 | 4.5 | 65.6 |
| 19.9 | 4.5 | 20.5 |
| 20.2 | 4.4 | 13.5 |
| 21.8 | 4.1 | 17.9 |
| 22.3 | 4.0 | 42.1 |
| 22.9 | 3.9 | 11.4 |
| 24.2 | 3.7 | 20.5 |
| 24.8 | 3.6 | 16.4 |
| 27.3 | 3.3 | 24.9 |

### Example 10—Alternative Preparation and Characterization of Crystalline Polymorphic Form A of 5,7-Dimethyl-N-((1S*4S)-4-(pentyloxy)cyclohexyl)pyrazolo[1,5-a]pyrimidine-3-carboxamide (Compound of Formula (VIII))

Crystalline polymorphic Form B of 5,7-dimethyl-N-((1S*,4S)-4-(pentyloxy)cyclohexyl)pyrazolo[1,5-a]pyrimidine-3-carboxamide (Compound of Formula (VIII) from Example 7, 750 g) was dissolved in 7.5 L of EtOAc at 40° C. The solution was passed through a CUNO filter, which was subsequently washed with additional EtOAc (2×7.5 L; 1×4 L). The colorless eluents were combined and passed through a 5 μm polishing filter. The line filter was washed with 2.0 L of EtOAc. All filtrates were combined and concentrated under reduced pressure to a total volume of approximately 1.7 L and then 1.85 LK of heptane was added. The resulting mixture was heated to 75° C. until all solids had dissolved. This solution was cooled to 50° C. over 30 min and then 1.5 g of crystalline polymorphic Form A of 5,7-dimethyl-N-((1S*,4S)-4-(pentyloxy)cyclohexyl)pyrazolo[1,5-a]pyrimidine-3-carboxamide was added as seed. The resulting mixture was stirred at 45° C. for 1 hour and then cooled to 20° C. over 20 min and stirred at this temperature for another 1 hour. Additional heptane (4.5 L) was added dropwise over 1 hour and the resulting suspension was aged overnight at 20° C. The slurry was filtered. The solids were washed with 1.5 L of 1:9 mixture of EtOAc and heptane, then with 0.75 L of heptane, and then dried overnight at 40° C. under vacuum to give 666 g of the title compound as a white solid (89% yield). The crystal form was confirmed as Form A by XRPD and DSC.

An X-ray powder diffractogram of the title compound is provided in FIG. 7. A differential scanning calorimetry curve of the title compound is provided in FIG. 8. The differential scanning calorimetry curve displayed an endothermic event at about 114° C. Tabulated characteristics of the X-ray powder diffractogram in FIG. 7 are provided below in Table 22, which lists diffraction angle 2θ and relative intensity (expressed as a percentage with respect to the most intense peak).

TABLE 22
X-RAY POWDER DIFFRACTOGRAM DATA OF
CRYSTALLINE POLYMORPHIC FORM A.

| Angle (2θ °) | Intensity % |
|---|---|
| 5.7 | 100 |
| 11.7 | 3.5 |
| 12.8 | 19.5 |
| 13.5 | 5.9 |
| 14.4 | 27.8 |
| 14.7 | 4.3 |
| 15.8 | 1.8 |
| 17.1 | 67.4 |
| 17.8 | 3.7 |
| 18.6 | 6.7 |
| 19.5 | 8.7 |
| 20.1 | 2.9 |
| 21.7 | 4.1 |
| 22.3 | 49.9 |
| 23.0 | 65.7 |
| 23.5 | 3.6 |
| 24.2 | 15.7 |
| 24.7 | 4.3 |
| 25.6 | 3.4 |
| 26.8 | 3.1 |
| 27.2 | 32.8 |
| 28.8 | 6.3 |
| 30.5 | 3.6 |
| 32.2 | 13.4 |
| 32.6 | 6.5 |

In addition, single crystals of crystalline polymorphic Form A of 5,7-dimethyl-N-((1S*,4S)-4-(pentyloxy)cyclohexyl)pyrazolo[1,5-a]pyrimidine-3-carboxamide were analyzed by single crystal X-ray diffraction analysis. The unit cell parameters of crystalline polymorphic Form A and the data collection and structure refinement methods are shown in Table 23 and Table 24, respectively.

TABLE 23
UNIT CELL PARAMETERS OF CRYSTALLINE
POLYMORPHIC FORM A.

| Empirical formula | C₂₀H₃₀N₄O₂ | |
|---|---|---|
| Formula weight | 358.48 | |
| Temperature | 100(2) K | |
| Wavelength | 1.54184 Å | |
| Crystal size | 0.220 × 0.200 × 0.100 mm | |
| Crystal habit | pale green cut block | |
| Crystal system | Monoclinic | |
| Space group | P2₁/c | |
| Unit cell dimensions | a = 15.8710(5) Å | α = 90° |
| | b = 9.4329(2) Å | β = 108.628(3)° |
| | c = 13.8255(4) Å | γ = 90° |

| | |
|---|---|
| Volume | 1961.38(10) Å³ |
| Z | 4 |
| Density (calculated) | 1.214 Mg/m³ |
| Absorption coefficient | 0.636 mm⁻¹ |
| F(000) | 776 |

TABLE 24
DATA COLLECTION AND STRUCTURE REFINEMENT METHODS
FOR CRYSTALLINE POLYMORPHIC FORM A.

| | |
|---|---|
| Diffractometer | SuperNova, Dual, Cu at zero, Atlas |
| Radiation source | SuperNova (Cu) X-ray Source, CuKα |
| Data collection method | omega scans |
| Theta range for data collection | 5.536 to 70.168° |
| Index ranges | −19 ≤ h ≤ 19, −11 ≤ k ≤ 10, −16 ≤ l ≤ 12 |
| Reflections collected | 19177 |
| Independent reflections | 3729 [R(int) = 0.0367] |
| Coverage of independent | 99.80% |
| reflections | |
| Variation in check reflections | N/A |
| Absorption correction | Semi-empirical from equivalents |
| Max. and min. transmission | 1.00000 and 0.92588 |
| Structure solution technique | Direct Methods |
| Structure solution program | SHELXTL (Sheldrick, 2014) |
| Refinement technique | Full-matrix least-squares on F² |
| Refinement program | SHELXL-2014 (Sheldrick, 2014) |
| Function minimized | Σw(Fₒ² − F_{c}²)² |
| Data/restraints/parameters | 3729/0/242 |
| Goodness-of-fit on F2 | 1.085 |
| D/smax | 0 |
| Final R indices | |
| 3140 data; I > 2 s(I) | R1 = 0.0424, wR2 = 0.1170 |
| all data | R1 = 0.0511, wR2 = 0.1242 |
| Weighting scheme | w = 1/[σ² (Fₒ²) + (0.0597P)² + 0.8324P] |
| | where P = (Fₒ² − 2F_{c}²)²/3 |
| Extinction coefficient | n/a |
| Largest diff. peak and hole | 0.271 and −0.201 eÅ⁻³ |

Atomic coordinates (×10⁴) and equivalent isotropic displacement parameters (Å²×10³) are shown in Table 25, below. U(eq) is defined as one third of the trace of the orthogonalized U^{ij} tensor.

TABLE 25
ATOMIC COORDINATES AND EQUIVALENT ISOTROPIC
ATOMIC DISPLACEMENT PARAMETERS FOR CRYSTALLINE
POLYMORPHIC FORM A.

| Atom | x/a | y/b | z/c | U(eq) |
|---|---|---|---|---|
| O1 | 0.45370(7) | 0.19892(12) | 0.55990(8) | 0.0266(3) |
| O2 | 0.14224(7) | 0.22366(11) | 0.09073(8) | 0.0231(3) |
| N1 | 0.18975(9) | 0.40009(14) | 0.20787(9) | 0.0217(3) |
| N2 | 0.15225(8) | 0.68936(13) | 0.13291(9) | 0.0174(3) |
| N3 | 0.08161(8) | 0.66673(13) | −0.04766(8) | 0.0173(3) |
| N4 | 0.05535(8) | 0.56549(14) | −0.12233(9) | 0.0207(3) |
| C1 | 0.67903(12) | 0.3036(2) | 1.01386(12) | 0.0324(4) |
| C2 | 0.63945(10) | 0.20816(18) | 0.92171(11) | 0.0268(4) |
| C3 | 0.60495(10) | 0.29103(17) | 0.82219(11) | 0.0248(3) |
| C4 | 0.55081(10) | 0.20047(18) | 0.73244(11) | 0.0255(3) |
| C5 | 0.50208(11) | 0.28940(17) | 0.64087(11) | 0.0267(4) |
| C6 | 0.39815(10) | 0.27481(17) | 0.47388(11) | 0.0226(3) |
| C7 | 0.30899(10) | 0.31283(18) | 0.48637(11) | 0.0258(3) |
| C8 | 0.25079(11) | 0.39347(18) | 0.39252(11) | 0.0266(4) |
| C9 | 0.23777(9) | 0.31100(15) | 0.29410(10) | 0.0190(3) |
| C10 | 0.32727(10) | 0.26462(18) | 0.28483(11) | 0.0242(3) |
| C11 | 0.38308(10) | 0.18422(18) | 0.37939(11) | 0.0263(3) |
| C12 | 0.15017(9) | 0.35113(15) | 0.11248(10) | 0.0178(3) |
| C13 | 0.11716(9) | 0.46188(16) | 0.03462(10) | 0.0181(3) |
| C14 | 0.11962(9) | 0.60824(15) | 0.04857(10) | 0.0167(3) |
| C15 | 0.14622(9) | 0.82833(15) | 0.12007(10) | 0.0176(3) |
| C16 | 0.10606(9) | 0.89203(16) | 0.02292(11) | 0.0191(3) |
| C17 | 0.07349(9) | 0.81007(16) | −0.06238(10) | 0.0186(3) |
| C18 | 0.07754(9) | 0.44396(16) | −0.07066(10) | 0.0201(3) |
| C19 | 0.18488(10) | 0.91867(16) | 0.21267(11) | 0.0229(3) |
| C20 | 0.03181(10) | 0.86192(17) | −0.16871(11) | 0.0240(3) |

Bond lengths (Å) are shown in Table 26, below.

TABLE 26
SELECTED BOND LENGTHS (Å)
FOR CRYSTALLINE POLYMORPHIC FORM A.

| Bond | Bond length (Å) | Bond | Bond length (Å) |
|---|---|---|---|
| O1—C5 | 1.4220(18) | O1—C6 | 1.4263(17) |
| O2—C12 | 1.2361(18) | N1—C12 | 1.3483(18) |
| N1—C9 | 1.4587(18) | N1—H1 | 0.86(2) |
| N2—C15 | 1.3223(19) | N2—C14 | 1.3520(18) |
| N3—C17 | 1.3673(19) | N3—N4 | 1.3699(17) |
| N3—C14 | 1.3876(17) | N4—C18 | 1.337(2) |
| C1—C2 | 1.522(2) | C2—C3 | 1.524(2) |
| C3—C4 | 1.525(2) | C4—C5 | 1.510(2) |
| C6—C11 | 1.515(2) | C6—C7 | 1.522(2) |
| C7—C8 | 1.533(2) | C8—C9 | 1.523(2) |
| C9—C10 | 1.530(2) | C10—C11 | 1.526(2) |
| C12—C13 | 1.4711(19) | C13—C14 | 1.393(2) |
| C13—C18 | 1.3993(19) | C15—C16 | 1.4228(19) |
| C15—C19 | 1.4966(19) | C16—C17 | 1.366(2) |
| C17—C20 | 1.4889(19) | | |

Bond angles (°) are shown in Table 27, below.

TABLE 27
SELECTED BOND ANGLES (°) FOR
CRYSTALLINE POLYMORPHIC FORM A.

| | Bond angle (°) | | Bond angle (°) |
|---|---|---|---|
| C5—O1—C6 | 112.94(12) | C12—N1—C9 | 124.10(13) |
| C12—N1—H1 | 117.2(12) | C9—N1—H1 | 118.4(12) |
| C15—N2—C14 | 117.00(12) | C17—N3—N4 | 125.85(11) |
| C17—N3—C14 | 121.83(12) | N4—N3—C14 | 112.32(12) |
| C18—N4—N3 | 103.31(11) | C1—C2—C3 | 112.67(14) |
| C2—C3—C4 | 113.19(13) | C5—C4—C3 | 112.06(13) |
| O1—C5—C4 | 109.26(13) | O1—C6—C11 | 108.74(12) |
| O1—C6—C7 | 111.90(12) | C11—C6—C7 | 109.53(12) |
| C6—C7—C8 | 110.59(12) | C9—C8—C7 | 112.07(13) |
| N1—C9—C8 | 108.80(12) | N1—C9—C10 | 110.66(12) |
| C8—C9—C10 | 110.86(12) | C11—C10—C9 | 111.75(12) |
| C6—C11—C10 | 110.31(13) | O2—C12—N1 | 123.40(13) |
| O2—C12—C13 | 121.87(12) | N1—C12—C13 | 114.72(13) |
| C14—C13—C18 | 104.31(13) | C14—C13—C12 | 127.95(12) |
| C18—C13—C12 | 127.74(13) | N2—C14—N3 | 122.10(13) |
| N2—C14—C13 | 131.80(13) | N3—C14—C13 | 106.08(12) |
| N2—C15—C16 | 122.47(13) | N2—C15—C19 | 117.21(12) |
| C16—C15—C19 | 120.31(13) | C17—C16—C15 | 120.52(13) |
| C16—C17—N3 | 116.07(12) | C16—C17—C20 | 126.35(14) |
| N3—C17—C20 | 117.57(13) | N4—C18—C13 | 113.98(13) |

Torsion angles (°) are shown in Table 28, below.

TABLE 28
SELECTED TORSION ANGLES (°)
FOR CRYSTALLINE POLYMORPHIC FORM A.

| | Torsion angle (°) | | Torsion angle (°) |
|---|---|---|---|
| C17—N3—N4—C18 | 179.61(13) | C14—N3—N4—C18 | 0.13(15) |
| C1—C2—C3—C4 | −169.88(13) | C2—C3—C4—C5 | 168.32(13) |
| C6—O1—C5—C4 | 173.76(12) | C3—C4—C5—O1 | 179.50(12) |
| C5—O1—C6—C11 | 154.81(13) | C5—O1—C6—C7 | −84.07(16) |
| O1—C6—C7—C8 | −179.69(12) | C11—C6—C7—C8 | −59.02(17) |
| C6—C7—C8—C9 | 55.76(18) | C12—N1—C9—C8 | −164.67(13) |
| C12—N1—C9—C10 | 73.29(17) | C7—C8—C9—N1 | −174.22(12) |
| C7—C8—C9—C10 | −52.31(18) | N1—C9—C10—C11 | 174.05(13) |
| C8—C9—C10—C11 | 53.24(17) | O1—C6—C11—C10 | −177.44(12) |
| C7—C6—C11—C10 | 59.99(17) | C9—C10—C11—C6 | −57.58(17) |
| C9—N1—C12—O2 | 8.2(2) | C9—N1—C12—C13 | −171.35(12) |
| O2—C12—C13—C14 | 178.28(14) | N1—C12—C13—C14 | −2.2(2) |
| O2—C12—C13—C18 | −3.0(2) | N1—C12—C13—C18 | 176.57(13) |
| C15—N2—C14—N3 | 0.22(19) | C15—N2—C14—C13 | 178.26(14) |
| C17—N3—C14—N2 | −1.26(19) | N4—N3—C14—N2 | 178.24(11) |
| C17—N3—C14—C13 | −179.74(12) | N4—N3—C14—C13 | −0.24(15) |
| C18—C13—C14—N2 | −178.04(14) | C12—C13—C14—N2 | 0.9(2) |
| C18—C13—C14—N3 | 0.23(14) | C12—C13—C14—N3 | 179.21(12) |
| C14—N2—C15—C16 | 1.10(19) | C14—N2—C15—C19 | −178.08(12) |
| N2—C15—C16—C17 | −1.5(2) | C19—C15—C16—C17 | 177.68(13) |
| C15—C16—C17—N3 | 0.42(19) | C15—C16—C17—C20 | −178.56(13) |
| N4—N3—C17—C16 | −178.56(12) | C14—N3—C17—C16 | 0.87(19) |
| N4—N3—C17—C20 | 0.5(2) | C14—N3—C17—C20 | 179.94(12) |
| N3—N4—C18—C13 | 0.03(15) | C14—C13—C18—N4 | −0.17(16) |
| C12—C13—C18—N4 | −179.15(13) | | |

Anisotropic displacement parameters (Å²) are shown in Table 29, below. The anisotropic displacement factor exponent may be expressed in the form: −2π²[h²a*²U¹¹+ . . . +2 h k a*b*U¹²].

TABLE 29
ANISOTROPIC DISPLACEMENT PARAMETERS (Å²) FOR CRYSTALLINE
POLYMORPHIC FORM A.

| Atom | U11 | U22 | U33 | U23 | U13 | U12 |
|---|---|---|---|---|---|---|
| O1 | 0.0271(6) | 0.0261(6) | 0.0203(5) | 0.0010(4) | −0.0013(4) | 0.0031(4) |
| O2 | 0.0298(6) | 0.0165(5) | 0.0219(5) | −0.0004(4) | 0.0068(4) | −0.0017(4) |
| N1 | 0.0305(7) | 0.0164(7) | 0.0152(6) | 0.0008(5) | 0.0030(5) | 0.0026(5) |
| N2 | 0.0194(6) | 0.0182(6) | 0.0141(5) | −0.0002(5) | 0.0047(4) | 0.0000(5) |
| N3 | 0.0196(6) | 0.0194(6) | 0.0123(5) | 0.0007(5) | 0.0043(4) | −0.0008(5) |
| N4 | 0.0245(6) | 0.0222(7) | 0.0147(6) | −0.0023(5) | 0.0054(5) | −0.0028(5) |
| C1 | 0.0327(9) | 0.0406(10) | 0.0221(8) | −0.0024(7) | 0.0063(7) | −0.0039(7) |
| C2 | 0.0259(8) | 0.0317(9) | 0.0214(8) | −0.0006(6) | 0.0056(6) | 0.0006(6) |
| C3 | 0.0219(7) | 0.0293(9) | 0.0222(8) | 0.0008(6) | 0.0054(6) | 0.0004(6) |
| C4 | 0.0230(7) | 0.0301(9) | 0.0213(7) | 0.0010(6) | 0.0042(6) | 0.0025(6) |
| C5 | 0.0256(8) | 0.0279(9) | 0.0220(7) | −0.0011(6) | 0.0013(6) | −0.0005(6) |
| C6 | 0.0237(7) | 0.0243(8) | 0.0170(7) | 0.0021(6) | 0.0024(6) | 0.0015(6) |
| C7 | 0.0296(8) | 0.0318(9) | 0.0152(7) | 0.0003(6) | 0.0061(6) | 0.0051(7) |
| C8 | 0.0303(8) | 0.0294(9) | 0.0185(7) | 0.0003(6) | 0.0052(6) | 0.0113(7) |
| C9 | 0.0225(7) | 0.0189(7) | 0.0149(7) | 0.0029(5) | 0.0048(5) | 0.0017(6) |
| C10 | 0.0254(8) | 0.0302(8) | 0.0174(7) | −0.0016(6) | 0.0074(6) | 0.0022(6) |
| C11 | 0.0237(7) | 0.0311(9) | 0.0225(8) | −0.0024(6) | 0.0050(6) | 0.0070(6) |
| C12 | 0.0197(7) | 0.0183(7) | 0.0160(7) | −0.0004(5) | 0.0067(5) | −0.0003(5) |
| C13 | 0.0192(7) | 0.0194(7) | 0.0159(7) | −0.0011(5) | 0.0059(5) | −0.0010(5) |
| C14 | 0.0176(6) | 0.0197(7) | 0.0131(6) | 0.0019(5) | 0.0053(5) | 0.0001(5) |
| C15 | 0.0176(7) | 0.0181(7) | 0.0176(7) | 0.0000(5) | 0.0064(5) | 0.0004(5) |
| C16 | 0.0201(7) | 0.0181(7) | 0.0191(7) | 0.0029(6) | 0.0065(5) | 0.0010(5) |
| C17 | 0.0169(7) | 0.0205(7) | 0.0189(7) | 0.0043(6) | 0.0062(5) | 0.0012(5) |
| C18 | 0.0225(7) | 0.0208(8) | 0.0173(7) | −0.0023(5) | 0.0066(5) | −0.0025(6) |
| C19 | 0.0310(8) | 0.0180(7) | 0.0182(7) | −0.0007(6) | 0.0058(6) | 0.0010(6) |
| C20 | 0.0262(7) | 0.0262(8) | 0.0176(7) | 0.0074(6) | 0.0041(6) | −0.0001(6) |

Hydrogen atom coordinates and isotropic atomic displacement parameters (Å²) are shown in Table 30, below.

TABLE 30
HYDROGEN ATOM COORDINATES AND ISOTROPIC
DISPLACEMENT PARAMETERS (Å²) FOR
CRYSTALLINE POLYMORPHIC FORM A.

| Atom | x/a | y/b | z/c | U |
|---|---|---|---|---|
| H1 | 0.1913(12) | 0.491(2) | 0.2163(13) | 0.025(5) |
| H1A | 0.726 | 0.3624 | 1.0026 | 0.049 |
| H1B | 0.7042 | 0.2451 | 1.0749 | 0.049 |
| H1C | 0.6324 | 0.3648 | 1.0234 | 0.049 |
| H2A | 0.59 | 0.1528 | 0.9319 | 0.032 |
| H2B | 0.6854 | 0.1403 | 0.9165 | 0.032 |
| H3A | 0.6561 | 0.3324 | 0.8058 | 0.03 |
| H3B | 0.5674 | 0.3702 | 0.8317 | 0.03 |
| H4A | 0.5911 | 0.134 | 0.7132 | 0.031 |
| H4B | 0.5072 | 0.1436 | 0.7535 | 0.031 |
| H5A | 0.4608 | 0.355 | 0.6589 | 0.032 |
| H5B | 0.5452 | 0.3466 | 0.6191 | 0.032 |
| H6 | 0.4291 | 0.3641 | 0.4655 | 0.027 |
| H7A | 0.3188 | 0.3723 | 0.548 | 0.031 |
| H7B | 0.2781 | 0.2252 | 0.4956 | 0.031 |
| H8A | 0.192 | 0.4124 | 0.4006 | 0.032 |
| H8B | 0.2789 | 0.4859 | 0.388 | 0.032 |
| H9 | 0.2013 | 0.2247 | 0.2947 | 0.023 |
| H10A | 0.3168 | 0.2032 | 0.2241 | 0.029 |
| H10B | 0.3605 | 0.3493 | 0.275 | 0.029 |
| H11A | 0.3522 | 0.0957 | 0.3867 | 0.032 |
| H11B | 0.4411 | 0.1584 | 0.3717 | 0.032 |
| H16 | 0.1019 | 0.9923 | 0.0173 | 0.023 |
| H18 | 0.0673 | 0.3536 | −0.1026 | 0.024 |
| H19A | 0.1438 | 0.9215 | 0.2526 | 0.034 |
| H19B | 0.1943 | 1.015 | 0.1916 | 0.034 |
| H19C | 0.2419 | 0.8786 | 0.2544 | 0.034 |
| H20A | 0.0679 | 0.8322 | −0.2109 | 0.036 |
| H20B | 0.0282 | 0.9656 | −0.1685 | 0.036 |
| H20C | −0.0281 | 0.8221 | −0.1967 | 0.036 |

Selected hydrogen bond information (Å and °) shown in Table 31, below.

TABLE 31
SELECTED HYDROGEN BOND FORMATION (Å and
°) FOR CRYSTALLINE POLYMORPHIC FORM A.

| D-H . . . A | d(D-H) | d(H . . . A) | d(D . . . A) | <(DHA) |
|---|---|---|---|---|
| N1—H1 . . . N2 | 0.86(2) | 2.18(2) | 2.9120(18) | 142.0(16) |

### Example 11—Preparation of Crystalline Polymorphic Form C of 5,7-Dimethyl-N-((1S*4S)-4-(pentyloxy)cyclohexyl)pyrazolo[1,5-a]pyrimidine-3-carboxamide (Compound of Formula (VIII))

Crystalline Form A 5,7-dimethyl-N-((1S*,4S)-4-(pentyloxy)cyclohexyl)pyrazolo[1,5-a]pyrimidine-3-carboxamide (500 mg) (Compound of Formula (VII) from Example 10) was dissolved in 5 mL of tert-butanol at 50° C. Upon addition of 5 mL of water a turbid mixture was produced which became clear after stirring for another 1 hour at 50° C. The resulting solution was quickly filtered using a 0.45 m PTFE filter and the clear filtrates were immediately frozen using dry ice/acetone batch. The resulting material was lyophilized for 20 hours to produce an off-white powder. The crystal form was confirmed as Form C by XRPD and DSC.

An X-ray powder diffractogram of the title compound is provided in FIG. 10. A differential scanning calorimetry curve of the title compound is provided in FIG. 11. Thermogravimetric analysis did not show a weight loss for this material. The differential scanning calorimetry curve displayed two endothermic events, one at about 110° C. and a second one at about 114° C. Tabulated characteristics of the X-ray powder diffractogram in FIG. 10 are provided below in Table 32, which lists diffraction angle 2θ and relative intensity (expressed as a percentage with respect to the most intense peak).

TABLE 32
X-RAY POWDER DIFFRACTOGRAM DATA OF
CRYSTALLINE POLYMORPHIC FORM C.

| Angle (2θ °) | Intensity % |
|---|---|
| 4.9 | 100 |
| 7.1 | 16.2 |
| 9.9 | 46.2 |
| 10.2 | 7.1 |
| 11.0 | 5.4 |
| 11.3 | 10.2 |
| 12.4 | 10.6 |
| 13.9 | 4.1 |
| 14.1 | 3.9 |
| 14.5 | 3.4 |
| 14.9 | 10.6 |
| 15.1 | 10.8 |
| 15.7 | 4.2 |
| 16.0 | 4.7 |
| 16.6 | 8.0 |
| 18.0 | 8.0 |
| 19.3 | 9.2 |
| 19.9 | 11.2 |
| 20.4 | 12.9 |
| 21.0 | 4.2 |
| 26.0 | 5.6 |
| 26.4 | 17 |
| 27.4 | 6.0 |

### Example 12—Preparation of Crystalline Hydrate Form D of 5,7-Dimethyl-N-((1S*,4S)-4-(pentyloxy)cyclohexyl)pyrazolo[1,5-a]pyrimidine-3-carboxamide (Compound of Formula (VIII))

A suspension of 1.0 g of crystalline Form B 5,7-dimethyl-N-((1S*,4S)-4-(pentyloxy)cyclohexyl)pyrazolo[1,5-a]pyrimidine-3-carboxamide (Compound of Formula (VIII) from Example 7) in 10 mL of water was stirred at 25° C. for 10 days and then filtered. The resulting off-white solids were confirmed to be crystalline hydrate Form D by XRPD and DSC.

An X-ray powder diffractogram of the title compound is provided in FIG. 12. A differential scanning calorimetry curve of the title compound is provided in FIG. 13. A thermogravimetric analysis showed a weight loss of approximately 6.8 wt % in the region between 0° C. to 120° C., which is equivalent to the loss of 1.5 mol equivalents of water. This was also confirmed by KF analysis of the solids. The differential scanning calorimetry curve displayed several endothermic events, culminating in a final event between about 108 and 115° C. The lower temperature endothermic events are associated with the loss of water and the final endothermic events with melting of Form A which suggests that upon loss of water the hydrate Form D converts to Form A under these conditions. Upon vacuum drying of hydrate D at ambient temperature the crystal from partially changes to that of Form C, a process which is accelerated at higher temperatures, also at atmospheric pressure. Tabulated characteristics of the X-ray powder diffractogram in FIG. 12 are provided below in Table 33, which lists diffraction angle 2θ and relative intensity (expressed as a percentage with respect to the most intense peak).

TABLE 33
X-RAY POWDER DIFFRACTOGRAM DATA OF
CRYSTALLINE POLYMORPHIC FORM D.

| Angle (2θ °) | Intensity % |
|---|---|
| 3.8 | 100 |
| 7.3 | 3.5 |
| 7.6 | 35.7 |
| 9.1 | 3.1 |
| 9.4 | 23.1 |
| 10.3 | 4.4 |
| 11.0 | 6.5 |
| 11.5 | 2.6 |
| 12.5 | 1.5 |
| 13.0 | 5.3 |
| 14.0 | 7.6 |
| 14.1 | 11.5 |
| 14.4 | 2.4 |
| 14.6 | 2.7 |
| 15.0 | 3.4 |
| 15.3 | 3.7 |
| 15.6 | 7.6 |
| 16.7 | 4.2 |
| 18.2 | 7.3 |
| 18.9 | 5.6 |
| 19.1 | 7.0 |
| 19.9 | 5.3 |
| 20.1 | 7.0 |
| 20.5 | 2.9 |
| 21.8 | 4.8 |
| 22.1 | 17.3 |
| 22.9 | 11.5 |
| 23.6 | 5.5 |
| 24.3 | 3.9 |
| 24.8 | 4.8 |
| 25.4 | 8.6 |
| 26.1 | 11.2 |
| 27.9 | 3.8 |
| 28.1 | 4.1 |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A compound in crystalline form having the following formula:

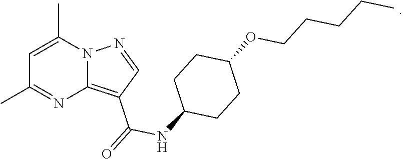 [SMILES: CCCCCO[C@H]1CC[C@H](NC(=O)c2cnn3c(C)cc(C)nc23)CC1]

2. The compound of claim 1, wherein the compound in crystalline form exhibits an X-ray powder diffraction pattern comprising peaks at the following diffraction angles (2θ): 5.7±0.2, 12.8±0.2, 14.4±0.2, and 17.1±0.2, or 5.7±0.2, 12.8±0.2, 14.4±0.2, 17.1±0.2, 22.3±0.2, 23.0±0.2 and 27.2±0.2.

3. The compound of claim 1, wherein the compound in crystalline form is characterized by the following X-ray powder diffraction pattern expressed in terms of diffraction angle 2θ:
Angle (2θ °)
5.7
11.7
12.8
13.5
14.4
14.7
15.8
17.1
17.8
18.6
19.5
20.1
21.7
22.3
23.0
23.5
24.2
24.7
25.6
26.8
27.2
28.8
30.5
32.2
32.6

4. The compound of anyone of claims 1-3, wherein the compound in crystalline form exists in a monoclinic crystal system and has a P2₁/c space group.

5. The compound of claim 4, wherein the compound in crystalline form is characterized by the following crystallographic unit cell parameters:

| | | |
|---|---|---|
| Unit cell | a = 15.8710(5) Å | α = 90° |
| dimensions | b = 9.4329(2) Å | β = 108.628(3)° |
| | c = 13.8255(4) | γ = 90° |
| | |
|---|---|
| Volume | 1961.38(10) Å³ |
| Z | 4 |
| Density | 1.214 Mg/m³ |
| (calculated) | |

6. The compound of any one of claims 1-5, wherein the compound in crystalline form is characterized by an X-ray powder diffraction pattern substantially the same as shown in FIG. 7.

7. The compound of any one of claims 1-6, wherein the compound has a melting point onset as determined by differential scanning calorimetry in the range of from about 112 degrees Celsius to about 116 degrees Celsius.

8. The compound of claim 7, wherein the compound has a melting point onset as determined by differential scanning calorimetry at about 114 degrees Celsius.

9. The compound of any one of claims 1-8, wherein the compound has a differential scanning calorimetry curve substantially the same as shown in FIG. 8.

10. The compound of claim 1, wherein the compound in crystalline form exhibits an X-ray powder diffraction pattern comprising peaks at the following diffraction angles (2θ): 4.2±0.2, 10.9±0.2, 11.5±0.2, and 12.4±0.2, or 4.2±0.2, 10.9±0.2, 11.5±0.2, 12.4±0.2, 16.3±0.2, 21.5±0.2, 22.3±0.2, 22.4±0.2, 22.9±0.2 and 23.0±0.2

11. The compound of claim 1 or 10, wherein the compound in crystalline form is characterized by the following X-ray powder diffraction pattern expressed in terms of diffraction angle 2θ:
Angle (2θ °)
4.2
10.9
11.5
12.4
13.5
14.8
16.3
17.7
18.6
19.5
20.2
20.4
21.1
21.5
21.8
22.3
22.4
22.9
23.0
23.5
23.8
24.7
25.8
27.6
28.3
29.8

12. The compound of any one of claims 1 and 10-11, wherein the compound in crystalline form exists in a monoclinic crystal system and has a P2₁/c space group.

13. The compound of claim 12, wherein the compound in crystalline form is characterized by the following crystallographic unit cell parameters:

| | | |
|---|---|---|
| Unit cell | a = 5.49080(10) Å | α = 90° |
| dimensions | b = 43.1070(8) Å | β = 94.827(2)° |
| | c = 8.2570(2) | γ = 90° |
| | |
|---|---|
| Volume | 1947.43(7) Å³ |
| Z | 4 |
| Density | 1.223 Mg/m³ |
| (calculated) | |

14. The compound of any one of claims 10-13, wherein the compound in crystalline form is characterized by an X-ray powder diffraction pattern substantially the same as shown in FIG. 3.

15. The compound of any one of claims 10-14, wherein the compound has a melting point onset as determined by differential scanning calorimetry in the range of from about 108 degrees Celsius to about 114 degrees Celsius.

16. The compound of claim 15, wherein the compound has a melting point onset as determined by differential scanning calorimetry at about 109 degrees Celsius.

17. The compound of any one of claims 1 and 10-16, wherein the compound has a differential scanning calorimetry curve substantially the same as shown in FIG. 4.

18. The compound of claim 1, wherein the compound in crystalline form exhibits an X-ray powder diffraction pattern comprising peaks at the following diffraction angles (2θ): 4.9±0.2, 7.1±0.2, 9.9±0.2, and 12.4±0.2, or 4.9±0.2, 7.1±0.2, 9.9±0.2, 12.4±0.2, 14.9±0.2, 15.1±0.2, 19.9±0.2, 20.4±0.2, and 26.4±0.2.

19. The compound of claim 1 or 18, wherein the compound in crystalline form is characterized by the following X-ray powder diffraction pattern expressed in terms of diffraction angle 2θ:
Angle (2θ °)
4.9
7.1
9.9
10.2
11.0
11.3
12.4
13.9
14.1
14.5
14.9
15.1
15.7
16.0
16.6
18.0
19.3
19.9
20.4
21.0
26.0
26.4
27.4

20. The compound of any one of claims 1 and 18-19, wherein the compound in crystalline form is characterized by an X-ray powder diffraction pattern substantially the same as shown in FIG. 10.

21. The compound of any one of claims 1 and 18-20, wherein the compound has a melting point onset as determined by differential scanning calorimetry in the range of from about 108 degrees Celsius to about 114 degrees Celsius.

22. The compound of claim 21, wherein the compound has a phase transition onset and a melting point onset, as determined by differential scanning calorimetry, at about 109 degrees Celsius and at about 113 degrees Celsius, respectively.

23. The compound of any one of claims 1 and 18-22, wherein the compound has a differential scanning calorimetry curve substantially the same as shown in FIG. 11.

24. The compound of claim 1, wherein the compound in crystalline form exhibits an X-ray powder diffraction pattern comprising peaks at the following diffraction angles (2θ): 3.8±0.2, 7.6±0.2, 9.4±0.2, and 14.1±0.2, or 3.8±0.2, 7.6±0.2, 9.4±0.2, 14.1±0.2, 22.1±0.2, 22.9±0.2, and 26.1±0.2.

25. The compound of claim 1 or 24, wherein the compound in crystalline form is characterized by the following X-ray powder diffraction pattern expressed in terms of diffraction angle 2θ:
Angle (2θ °)
3.8
7.3
7.6
9.1
9.4
10.3
11.0
11.5
12.5
13.0
14.0
14.1
14.4
14.6
15.0
15.3
15.6
16.7
18.2
18.9
19.1
19.9
20.1
20.5
21.8
22.1
22.9
23.6
24.3
24.8
25.4
26.1
27.9
28.1

26. The compound of any one of claims 1 and 24-25, wherein the compound in crystalline form is characterized by an X-ray powder diffraction pattern substantially the same as shown in FIG. 12.

27. The compound of any one of claims 1 and 24-26, wherein the compound has a melting point onset as determined by differential scanning calorimetry in the range of from about 108 degrees Celsius to about 114 degrees Celsius.

28. The compound of claim 27, wherein the compound has a melting point onset as determined by differential scanning calorimetry at about 109 degrees Celsius.

29. The compound of any one of claims 1 and 24-28, wherein the compound has a differential scanning calorimetry curve substantially the same as shown in FIG. 13.

30. A pharmaceutical composition, comprising a compound of any one of claims 1-29 and a pharmaceutically acceptable carrier.

31. A method of treating a disorder selected from the group consisting of Gaucher disease, Parkinson's disease, Lewy body disease, dementia, multiple system atrophy, epilepsy, bipolar disorder, schizophrenia, an anxiety disorder, major depression, polycystic kidney disease, type 2 diabetes, open angle glaucoma, multiple sclerosis, endometriosis, and multiple myeloma, comprising administering to a patient in need thereof a therapeutically effective amount of a compound of any one of claims 1-29 to treat the disorder.

32. The method of claim 31, wherein the disorder is Gaucher disease.

33. The method of claim 31, wherein the disorder is Parkinson's disease.

34. The method of claim 31, wherein the disorder is Lewy body disease.

35. The method of claim 31, wherein the disorder is dementia.

36. The method of claim 31, wherein the disorder is multiple system atrophy.

37. The method of any one of claims 31-36, wherein the patient is a human.

38. The method of any one of claims 31-37, wherein the compound is a compound of claim 2.

39. The method of any one of claims 31-37, wherein the compound is a compound of claim 5.

40. The method of any one of claims 31-37, wherein the compound is a compound of claim 10.

41. The method of any one of claims 31-37, wherein the compound is a compound of claim 13.

42. The method of any one of claims 31-37, wherein the compound is a compound of claim 18.

43. The method of any one of claims 31-37, wherein the compound is a compound of claim 19.

44. The method of any one of claims 31-37, wherein the compound is a compound of claim 24.

45. The method of any one of claims 31-37, wherein the compound is a compound of claim 25.

46. A method of preparing a compound, the method comprising:
(a) admixing a compound of Formula (I), a base, and a solvent to produce a reaction mixture; wherein Formula (I) is represented by: 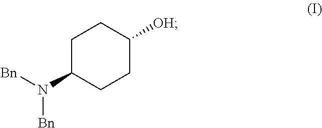 [SMILES: O[C@H]1CC[C@H](N(Cc2ccccc2)Cc2ccccc2)CC1]
(b) adding a n-pentyl alkylating agent to the reaction mixture to produce a compound of Formula (II): 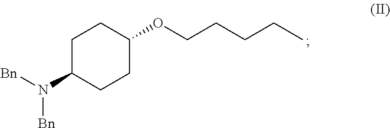 [SMILES: CCCCCO[C@H]1CC[C@H](N(Cc2ccccc2)Cc2ccccc2)CC1]
(c) exposing the compound of Formula (II) to acid HX to provide a compound of Formula (III): 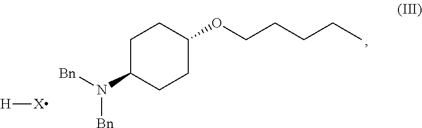
wherein X is an anion; and
(d) exposing the compound of Formula (III) to hydrogenation conditions, to provide a compound of Formula (IV): 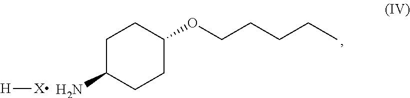
wherein X is an anion.

47. The method of claim 46, wherein the base in step (a) is a metal hydride, a metal carbonate, a metal bicarbonate, or metal alkoxide.

48. The method of claim 46, wherein the base in step (a) is a metal hydride or metal alkoxide.

49. The method of claim 46, wherein the base in step (a) is sodium hydride or potassium t-butoxide.

50. The method of any one of claims 46-49, wherein the solvent in step (a) is a polar, aprotic organic solvent.

51. The method of any one of claims 46-50, wherein the solvent in step (a) is dimethylacetamide, dimethylformamide, dimethylsulfoxide, diethyl ether, tetrahydrofuran, 1,4-dioxane, or a mixture thereof.

52. The method of any one of claims 46-51, wherein the solvent in step (a) is dimethylacetamide or dimethylsulfoxide.

53. The method of any one of claims 46-51, wherein the solvent in step (a) is dimethylsulfoxide, tetrahydrofuran, or a mixture thereof.

54. The method of any one of claims 46-53, wherein the n-pentyl alkylating agent is n-pentyl bromide.

55. The method of any one of claims 46-54, wherein the temperature of the reaction mixture in steps (a) and (b) is independently less than about 35 degrees Celsius.

56. The method of any one of claims 46-55, wherein acid HX in step (c) is a mineral acid.

57. The method of any one of claims 46-55, wherein acid HX in step (c) is hydrochloric acid, hydrobromic acid, sulfuric acid, nitric acid, or phosphoric acid.

58. The method of any one of claims 46-55, wherein acid HX in step (c) is hydrochloric acid.

59. The method of any one of claims 46-55, wherein acid HX in step (c) is an organic carboxylic acid compound.

60. The method of any one of claims 46-59, wherein exposing the compound of Formula II to acid HX in step (c) comprises adding to the compound of Formula II a solution containing acid HX and a (C₁₋₄ alkyl)-CO₂—(C₁₋₄ alkyl) solvent.

61. The method of any one of claims 46-59, wherein exposing the compound of Formula II to acid HX in step (c) comprises adding to the compound of Formula II a solution containing acid HX and ethyl acetate.

62. The method of any one of claims 46-61, wherein the hydrogenation conditions in step (d) comprise a hydrogenation catalyst and a hydrogen source.

63. The method of claim 62, wherein the hydrogenation catalyst is palladium hydroxide on carbon, palladium on carbon, or Raney nickel.

64. The method of claim 62, wherein the hydrogenation catalyst is palladium hydroxide on carbon.

65. The method of claim 62, wherein the hydrogenation catalyst is palladium on carbon.

66. The method of any one of claims 62-65, wherein the hydrogen source is hydrogen gas, ammonium formate, or cyclohexene.

67. The method of any one of claims 62-65, wherein the hydrogen source is hydrogen gas.

68. The method of any one of claims 62-67, wherein the hydrogenation conditions further comprise a solvent containing an alcohol, an ether, or a mixture thereof.

69. The method of claim 68, wherein the solvent is a saturated aliphatic alcohol.

70. The method of claim 68, wherein the solvent is methanol.

71. The method of any one of claims 62-67, wherein hydrogenation conditions are performed at about atmospheric pressure at a temperature in the range of from about 20 degrees Celsius to about 25 degrees Celsius.

72. The method of any one of claims 46-71, further comprising admixing a compound of Formula (V) and benzyl bromide in the presence of a base (B) and a solvent (S) to produce a compound of Formula I, wherein Formula (V) is represented by:

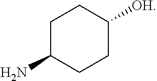

73. The method of claim 72, wherein base (B) is potassium carbonate, potassium bicarbonate, sodium carbonate, sodium bicarbonate, cesium carbonate, or cesium bicarbonate.

74. The method of claim 72, wherein base (B) is potassium carbonate.

75. The method of any one of claims 72-74, wherein solvent (S) is a polar, aprotic organic solvent.

76. The method of any one of claims 72-74, wherein solvent (S) is dimethylformamide, dimethylacetamide, dimethylsulfoxide, tetrahydrofuran, diethyl ether, or 1,4-dioxane.

77. The method of any one of claims 72-74, wherein solvent (S) is dimethylformamide.

78. The method of any one of claims 72-77, wherein the step to produce a compound of Formula I is performed at a temperature less than about 35 degrees Celsius.

79. The method of any one of claims 46-78, further comprising admixing a compound of Formula (VII) with an amide coupling reagent in the presence of a solvent (S1) to form an amide-coupling reaction mixture, and thereafter adding a compound of Formula (IV) to the amide-coupling reaction mixture, to provide a mixture containing a compound of Formula (VIII), wherein the compound of Formula (IV) is represented by 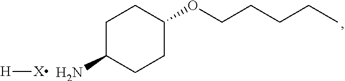
wherein X is an anion, the compound of Formula (VII) is represented by 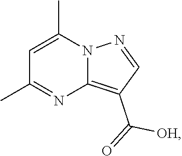
and the compound of Formula (VIII) is represented by:

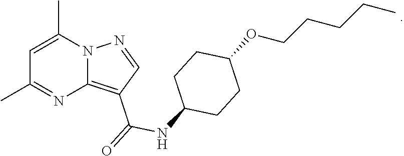 [SMILES: CCCCCO[C@H]1CC[C@H](NC(=O)c2cnn3c(C)cc(C)nc23)CC1]

80. The method of claim 79, wherein the amide-coupling reagent comprises a uronium amide-coupling reagent, a phosphonium amide-coupling reagent, or a carbodiimide.

81. The method of claim 79, wherein the amide-coupling reagent comprises O-(7-azabenzotriazol-1-yl)-N,N,N′,N′-tetramethyluronium hexafluorophosphate (HATU) or 1-ethyl-3-(3-dimethylaminopropyl)carbodiimide hydrochloride (EDC).

82. The method of claim 80 or 81, wherein the amide-coupling reagent further comprises a base.

83. The method of claim 80 or 81, wherein the amide-coupling reagent further comprises diisopropylethylamine, triethylamine, or N-methylmorpholine.

84. The method of claim 80 or 81, wherein the amide-coupling reagent further comprises diisopropylethylamine (DIPEA).

85. The method of any one of claims 79-84, wherein an additive is added to the coupling reaction to accelerate the reaction.

86. The method of claim 85, wherein the additive is 2-hydroxypyridine-N-oxide (HOPO).

87. The method of any one of claims 79-86, wherein solvent (S1) is a polar, aprotic organic solvent.

88. The method of any one of claims 79-86, wherein solvent (S1) comprises dimethylformamide, dimethylacetamide, or dimethylsulfoxide.

89. The method of any one of claims 79-86, wherein solvent (S1) comprises dimethylformamide.

90. The method of any one of claims 79-89, wherein the temperature of the amide-coupling reaction mixture is less than about 30 degrees Celsius.

91. The method of any one of claims 79-90, further comprising adding water to the mixture containing a compound of Formula (VIII), to provide the compound of Formula (VIII) in the form of a crystalline solid.

92. The method of claim 91, wherein the volume of water added is in the range of about 0.5 to about 3 times the volume of the mixture containing a compound of Formula (VIII).

93. The method of claim 91, wherein the volume of water added is approximately equal to the volume of the mixture containing a compound of Formula (VIII).

94. The method of any one of claims 91-93, further comprising the steps of:
(i) isolating the compound of Formula (VIII) in the form of a crystalline solid, to thereby provide an isolated crystalline compound of Formula (VIII); and
(ii) washing the isolated crystalline compound of Formula (VIII) one or more times with a solvent (S2) comprising water and dimethylformamide where the ratio of volume of water to dimethylformamide in solvent (S2) is in the range of 3:1 to 5:1, to provide a purified isolated crystalline compound of Formula (VIII).

95. The method of claim 94, wherein the purified isolated crystalline compound of Formula (VIII) exhibits an X-ray powder diffraction pattern comprising peaks at the following diffraction angles (2θ): 5.7±0.2, 12.8±0.2, 14.4±0.2, and 17.1±0.2, or 5.7±0.2, 12.8±0.2, 14.4±0.2, 17.1±0.2, 22.3±0.2, 23.0±0.2 and 27.2±0.2.

96. The method of claim 94, wherein the purified isolated crystalline compound of Formula (VIII) exhibits an X-ray powder diffraction pattern comprising peaks at the following diffraction angles (2θ): 4.2±0.2, 10.9±0.2, 11.5±0.2, and 12.4±0.2, 4.2±0.2, 10.9±0.2, 11.5±0.2, 12.4±0.2, 16.3±0.2, 21.5±0.2, 22.3±0.2, 22.4±0.2, 22.9±0.2 and 23.0±0.2.

97. The method of claim 94, wherein the purified isolated crystalline compound of Formula (VIII) exhibits an X-ray powder diffraction pattern comprising peaks at the following diffraction angles (2θ): 3.8±0.2, 7.6±0.2, 9.4±0.2, and 14.1±0.2, or 3.8±0.2, 7.6±0.2, 9.4±0.2, 14.1±0.2, 22.1±0.2, 22.9±0.2, and 26.1±0.2.

98. The method of any one of claims 79-90, further comprising the steps of:
(i) isolating the compound of Formula (VIII) in the form of a solid, to thereby provide an isolated compound of Formula (VIII);
(ii) dissolving the isolated compound of Formula (VIII) in solvent selected from the group consisting of a (C₁₋₄ alkyl)-CO₂—(C₁₋₄ alkyl) ester, a saturated aliphatic alcohol or a (C₁₋₄ alkyl)-CO—(C₁₋₄ alkyl) ketone at a temperature of from about 20 degrees Celsius to about 50 degrees Celsius, thereby forming a mixture;
(iii) adding an C₅₋₈ alkane solvent to the mixture of step (ii) and allowing the mixture to cool to a temperature of from about 0 degrees Celsius to about 25 degrees Celsius;
(iv) aging the mixture of step (iii) to provide a compound of Formula (VIII) in the form of a crystalline solid; and
(v) isolating the compound of Formula (VIII) in the form of a crystalline solid to provide a first isolated crystalline compound of Formula (VIII).

99. The method of claim 98, wherein the solvent is step (ii) is a (C₁₋₄ alkyl)-CO—(C₁₋₄ alkyl) ketone.

100. The method of claim 99, wherein the (C₁₋₄ alkyl)-CO—(C₁₋₄ alkyl) is methyl ethyl ketone.

101. The method of any one of claims 98-100, wherein the C₅₋₈ alkane is heptane.

102. The method of any one of claims 98-101, wherein the first isolated crystalline compound of Formula (VIII) exhibits an X-ray powder diffraction pattern comprising peaks at the following diffraction angles (2θ): 4.2±0.2, 10.9±0.2, 11.5±0.2, and 12.4±0.2, or 4.2±0.2, 10.9±0.2, 11.5±0.2, 12.4±0.2, 16.3±0.2, 21.5±0.2, 22.3±0.2, 22.4±0.2, 22.9±0.2 and 23.0±0.2.

103. The method of any one of claims 98-102, further comprising the steps of:
(i) dissolving the first isolated crystalline compound of Formula (VIII) in ethyl acetate at a temperature of about 40 degrees Celsius, thereby forming a mixture;
(ii) adding heptane to the mixture of step (i) and heating the mixture to a temperature of about 75 degrees Celsius;
(iii) cooling the mixture of step (ii) to a temperature of about 50 degrees Celsius and adding seeds of a second isolated crystalline compound of Formula (VIII), thereby producing a seeded mixture;
(iv) aging the seeded mixture of step (iii) to provide a compound of Formula (VIII) in the form of a second crystalline solid; and
(v) isolating the compound of Formula (VIII) in the form of a crystalline solid to provide the second isolated crystalline compound of Formula (VIII).

104. The method of claim 103, wherein the second isolated crystalline compound of Formula (VIII) exhibits an X-ray powder diffraction pattern comprising peaks at the following diffraction angles (2θ): 5.7±0.2, 12.8±0.2, 14.4±0.2, and 17.1±0.2, or 5.7±0.2, 12.8±0.2, 14.4±0.2, 17.1±0.2, 22.3±0.2, 23.0±0.2 and 27.2±0.2.

105. The method of any one of claims 103-104, further comprising the steps of:
(i) dissolving the second isolated crystalline compound of Formula (VIII) in a water miscible solvent at a temperature of about 50 degrees Celsius, thereby forming a mixture;
(ii) adding water to the mixture of step (i); and
(iii) isolating the compound of Formula (VIII) in the form of a crystalline solid to provide a third isolated crystalline compound of Formula (VIII).

106. The method of claim 105, wherein the water miscible solvent is t-butanol.

107. The method of claim 105 or 106, wherein the third isolated crystalline compound of Formula (VIII) exhibits an X-ray powder diffraction pattern comprising peaks at the following diffraction angles (2θ): 4.9±0.2, 7.1±0.2, 9.9±0.2, and 12.4±0.2, or 4.9±0.2, 7.1±0.2, 9.9±0.2, 12.4±0.2, 14.9±0.2, 15.1±0.2, 19.9±0.2, 20.4±0.2, and 26.4±0.2.

108. The method of any one of claims 98-102, further comprising the steps of:
(i) adding the first isolated crystalline compound of Formula (VIII) to water, thereby forming a mixture;
(ii) aging the mixture of step (i); and
(iii) isolating the compound of Formula (VIII) in the form of a crystalline solid to provide a fourth isolated crystalline compound of Formula (VIII).

109. The method of claim 108, wherein the first isolated crystalline compound of Formula (VIII) is added to water at a temperature of from about 20 degrees Celsius to about 40 degrees Celsius.

110. The method of claim 108, wherein the third isolated crystalline compound of Formula (VIII) exhibits an X-ray powder diffraction pattern comprising peaks at the following diffraction angles (2θ): 3.8±0.2, 7.6±0.2, 9.4±0.2, and 14.1±0.2, or 3.8±0.2, 7.6±0.2, 9.4±0.2, 14.1±0.2, 22.1±0.2, 22.9±0.2, and 26.1±0.2.
